(19) United States
(12) Patent Application Publication
(10) Pub. No.: US 2022/0049275 A1
(43) Pub. Date: Feb. 17, 2022

(54) SSI CELLS WITH PREDICTABLE AND STABLE TRANSGENE EXPRESSION AND METHODS OF FORMATION

(71) Applicants: LONZA, LTD., VISP (CH); BABRAHAM INSTITUTE ENTERPRISE, LTD., CAMBRIDGE (GB); PFIZER, INC., SAN FRANCISCO, CA (US)

(72) Inventors: PETER M. O'CALLAGHAN, Slough (GB); STEPHEN BEVAN, Cambridge (GB); ROBERT YOUNG, Slough (GB); PETER FRASER, TALLAHASSEE, FL (US); LIN ZHANG, SAN FRANCISCO, CA (US)

(21) Appl. No.: 17/278,866

(22) PCT Filed: Oct. 1, 2019
(86) PCT No.: PCT/US2019/054045

§ 371 Date: Mar. 23, 2021

Related U.S. Application Data
(60) Provisional application No. 62/739,546, filed on Oct. 1, 2018.

(51) Int. Cl. C12N 15/90

(52) U.S. Cl. CPC C12N 15/907

(57) ABSTRACT

Mammalian cells are described that includes a recombination target site integrated within high integrating locus. Recombinant protein producer cell lines incorporating the mammalian cells and methods for forming the mammalian cells are also described. The high integrating loci have been developed through understanding and mapping of the three dimensional hierarchical structure of chromatin in mammalian cells. The high integrating loci are present in transcriptionally active environments that can provide both chromatin accessibility and epigenetic stability. As such, the recombinant mammalian cells can provide predictable and stable transgene production.

### CROSS REFERENCE TO RELATED APPLICATION

This application claims filing benefit of U.S. Provisional Patent Application Ser. No. 62/739,546, having a filing date of Oct. 1, 2018, which is incorporated herein by reference for all purposes.

### BACKGROUND

Integration of a recombinant protein (rP) expression cassette in a host cell for expression of heterologous polypeptides has been carried out for many years. Traditionally, random integration (RI) processes were used that take advantage of existing double strand breaks in the genome for incorporation of the expression cassette. Unfortunately, due to the position variegation effect, both the number of gene copies integrated and the expression characteristics at the integration sites can be highly variable in RI processes, giving rise to undesirable phenotypic heterogeneity. As such, RI processes require expensive screening of integration events in development of a useful cell line. Moreover, gene amplification methods that are used to increase expression can give rise to instability in the genome (e.g., deletions, duplications, translocations) as well as expression-modifying epigenetic actions (e.g., methylation, histone modification, heterochromatin invasion). As a result, RI-produced cell lines are often unstable and show reduced production over time.

More recently, site-specific integration (SSI) has been developed in which “landing pads” are formed in the cell genome through integration of recombination target sites (RTS) derived from site-specific recombinase systems such as the Saccharomyces cerevisiae-derived FLP-Frt system or the bacteriophage P1 derived Cre-loxP system. The process of integrating cassettes in SSI cell lines is referred to as recombinase-mediated cassette-exchange (RMCE). RMCE generally involves co-transfection of an expression vector encoding the recombinase along with a targeting expression vector containing the gene of interest (GOI) flanked by recombinase targeting sequences. By using distinct RTS at the 5′ and 3′ ends of the cassette to be exchanged (in both donor and target DNA) the SSI integration approach can ensure that the recombination occurs in a directional manner and that only the preferred cassette region is exchanged.

Unfortunately, SSI-generated cell lines can also have limitations. For instance, SSI systems require insertion of the RTS into the genome as a prerequisite for vector targeting and generation of cell lines expressing the GOI. The RTS insertion is generally carried out by RI or into a limited number of specific genomic regions, and thus the resulting cell lines are still subject to instability and reduced production over time. Moreover, SSI generally results in a low number of integrated gene copies that could indirectly limit rP production titres.

One method to increase integrated copies of recombinant genes is referred to as cumulative or accumulative SSI (see e.g., Kameyama et al. Biotechnol. Bioeng. 105:1106-14 (2010), Kawabe et al. Cytotechnology 64:267-79 (2012) and Turan et al. J. Mol. Biol. 402:52-69 (2010)). Such a method can include repeated rounds of RMCE to load up a single site sequentially with multiple copies of rP expression cassettes.

What are needed in the art are SSI cell lines that incorporate RTS at transcriptionally active and highly stable loci within the genome of the host cell. Such cell lines would be capable of stable and long-term expression of GOI.

Publications, patents, and patent applications are cited herein, the disclosures of which are incorporated by reference herein in their entireties.

### SUMMARY

The present disclosure is based upon the recognition that the transcriptional output from a transgene insertion site as well as the stability of the expression system thereof will be strongly influenced by the 3-dimensional (3D) structure of the chromatin in that region. The present disclosure describes methods based on this recognition for determination of the structure and confirmation of a genome in 3 dimensions (3D mapping of a genome). The disclosed 3D mapping methods can be carried out through utilization of techniques such as, e.g., Hi-C and other chromosome conformation capture methods (Elzo de Wit and Wouter de Laat. Genes Dev. 2012 26: 11-24) and Promoter Capture Hi-C (Schoenfelder et al. Genome Res 25:582-97 (2015), among others. Methods of utilizing information obtained by the 3D mapping protocols as well as mammalian cells that can be formed by the methods are also described. This application teaches how to generate multi-level 3D genome maps and then use that information to identify optimal genome integration sites for the expression of heterologous genes. For example, by interrogating the mapped 3D genome structure, integration sites likely to exhibit high performance can be identified.

In one embodiment, the present disclosure is directed to a mammalian cell that includes an RTS at a high integrating (HI) locus. HI loci are high performance genomic sites identified by the inventors through analysis of the 3D hierarchical structure of genomic chromatin. Beneficially, HI loci are in stable, transcriptionally active environments of the genome and can be repeatedly targeted to deliver predictable and stable levels of GOI expression.

HI loci can be within an active genomic compartment of accessible chromatin and can also be within about 30,000 base pairs of a topologically associated domain (TAD) boundary. In addition, HI loci can overlap regions of the genome that interact with at least one enhancer element. HI loci can vary depending on whether expression of the GOI will be driven by an in situ endogenous promoter or by a heterologous promoter. For instance, in those cell lines in which expression of the GOI is driven by an in situ endogenous promoter, HI loci can overlap and be downstream of a transcription start site (TSS). Moreover, in this embodiment, HI loci can overlap an active, and in some embodiments, also fully annotated gene loci, e.g., an active gene the expression product of which or lack thereof is non-vital to the cell. In those cell lines in which expression of the GOI is driven by a heterologous promoter, HI loci can generally be external to active or non-transcribed gene loci. For example, HI loci in such a cell can encompass loci that do not overlap any associated promoter regions of active genes or in one embodiment that do not come within about 1,000 base pairs of any active gene (e.g., within about 1,000 base pairs of any active and fully annotated gene).

In some embodiments, a cell can include multiple RTS, e.g., at least two RTS, at least four RTS, or even more in some embodiments. For instance, a cell can include multiple RTS in a single HI locus, in distinct HI loci, and/or in separate loci (e.g., the FerIL4 locus).

In some embodiments, an RTS can include an Frt site, a lox site, a rox site, or an att site. In some embodiments, an RTS can include a sequence selected from among SEQ ID Nos.: 126-155.

Cell types encompassed herein can include, without limitation, a mouse cell, a human cell, a Chinese hamster ovary (CHO) cell, a CHO-K1 cell, a CHO-DXB11 cell, a CHO-DG44 cell, a CHOK1SV™ cell including all variants, a CHO glutamine synthetase knockout cell including all variants, a HEK cell, a HEK293 cell including adherent and suspension-adapted variants, a HeLa cell, or a HT1080 cell.

In one embodiment, a cell can include a GOI, e.g., a chromosomally integrated GOI such as a reporter gene, a selection gene, a gene of therapeutic interest, an ancillary gene, or a combination of genes. A GOI can encode a difficult to express (DtE) protein such as an Fc-fusion protein, an enzyme, a membrane receptor, or a monoclonal antibody (e.g., a bi-specific or a tri-specific monoclonal antibody). In one embodiment, a GOI can be located between two RTS within a single HI locus. A cell can incorporate multiple GOI in some embodiments. For instance, a cell can incorporate two or more GOI within a single HI locus, can incorporate multiple GOI, one or more of which being in different HI loci, and/or can incorporate multiple GOI in any combination of HI loci and separate loci. In some embodiments, a cell can incorporate a recombinase gene, for instance a site-specific recombinase gene that in one embodiment can be chromosomally integrated.

Also disclosed are methods for producing a recombinant cell. For instance, a method can include mapping peaks in accessible chromatin of a cell genome and identifying within the mapped peaks in accessible chromatin a first set of peaks that are within active genomic compartments of the accessible chromatin and also within about 30,000 base pairs of a topologically associated domain (TAD) boundary. In one embodiment, the first set of peaks can be within active genomic compartments (for instance, as defined by Principle Component Analysis Methods (PCA)) and can also be within open chromatin (for instance, as defined by ATAC-seq), but this is not a requirement of a method, and in other embodiments, the first set of peaks can include those peaks that are within active genomic compartments within the whole of the mapped accessible chromatin. The method can also include identifying among the first set of peaks those that overlap regions of the genome that interact with at least one enhancer element. An HI locus can then be defined among the peaks that fit these criteria. Following identification of an HI locus, an RTS can be inserted into the HI locus. Optionally, a gene encoding a site-specific recombinase can also be inserted into the cell.

In those embodiments in which expression of a gene from the HI locus is to be driven by an in situ endogenous promoter, a method can further include identifying among the first set of peaks that overlap regions of the genome that interact with at least one enhancer element a second set of peaks that overlap a TSS, and in particular TSS for active genes the expression product of which or lack thereof is non-vital. The HI locus can be defined within this second set of peaks, the HI locus overlapping an active gene and being downstream of the TSS of the active gene.

In those embodiments in which expression of a gene from the HI locus is to be driven by a heterologous promoter, a method can further include identifying within the first set of peaks that overlap regions of the genome that interact with at least one enhancer element those peaks within accessible chromatin that do not overlap active genes or their associated promoter regions and an HI locus can be defined within this second set of peaks.

A method can also include transfecting the cell with a vector that includes an exchangeable cassette encoding a GOI and integrating the exchangeable cassette into an HI locus. A cell that includes the exchangeable cassette integrated into the chromosome at an HI locus can then be selected as a recombinant protein producer cell.

Optionally, methods can include incorporating additional RTS into the cell. For instance, additional RTS can be incorporated into the same HI locus as the first RTS, into one or more additional HI loci, and/or into one or more separate loci.

According to another embodiment, a method for producing a recombinant cell is disclosed that includes mapping peaks in accessible chromatin of a cell genome and identifying within the mapped peaks in accessible chromatin a first set of peaks that are within active genomic compartments of the accessible chromatin and also within about 30,000 base pairs of a topologically associated domain (TAD) boundary. In one embodiment, the first set of peaks can be within active genomic compartments (for instance, as defined by Principle Component Analysis Methods (PCA)) and can also be within open chromatin (for instance, as defined by ATAC-seq), but this is not a requirement of a method, and in other embodiments, the first set of peaks can include those peaks that are within active genomic compartments within the whole of the mapped accessible chromatin. The method can also include identifying within the first set of peaks those that overlap regions of the genome that interact with at least one enhancer element. A plurality of HI loci can then be defined within the resulting set of mapped peaks. A method can further include integrating an RTS into a plurality of cells (e.g., according to an RI protocol), and then selecting from that plurality of cells a cell comprising the RTS integrated into an HI locus. Optionally, a gene encoding a site-specific recombinase can also be inserted into that selected cell.

In one embodiment, the HI loci identified by the method can be ranked according to effectiveness. For instance, the HI loci can be ranked according to one or more of the expression level of one or more genes associated with each locus, the distance from each locus to the nearest TAD boundary, and the number of predicted enhancer interactions of each locus. In one such embodiment, in which a cell is selected that includes the RTS integrated into an HI locus, the cell(s) can be selected according to the ranking of the HI locus insertions sites.

In one embodiment, the method of defining the HI loci can also depend upon whether the HI loci are intended to be utilized to express a heterologous gene driven with an in situ endogenous promoter or a heterologous promoter. For instance, in those embodiments in which expression of genes from the HI loci is to be driven by an in situ endogenous promoter, a method can further include identifying within the resulting set of mapped peaks as defined above those peaks that overlap a TSS for active genes, such as an active gene the expression product of which or lack thereof is non-vital. A second set of peaks can then be defined that overlap the identified genes and that are downstream of the TSS of these identified genes, and the HI loci can be defined within this second set of peaks.

In those embodiments in which expression of genes from the HI loci is to be driven by a heterologous promoter, a method can further include identifying within the resulting set of mapped peaks as defined above a second set of peaks that do not overlap any genes, e.g., any active genes, or their associated promoter regions and the HI loci can be defined within this second set of peaks.

A method can also include transfecting a selected cell that includes an RTS integrated into an HI locus with a vector that includes an exchangeable cassette encoding a GOI and integrating the exchangeable cassette into the HI locus. A cell that includes the exchangeable cassette integrated into the chromosome can then be selected as a recombinant protein producer cell.

Optionally, methods can include incorporating additional RTS into the cell. For instance, additional RTS can be incorporated into a first HI locus, into one or more additional HI loci, and/or into one or more separate loci.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figure in which:

FIG. 1 presents a flow chart showing one embodiment of methods for production of a 3D map of a genome and utilization thereof to define and rank candidate HI loci. The diagram shows a summary of sequential filtering or screening process by which the data used to generate the multi-level 3D genome map can then be used to identify candidate HI loci.

FIG. 2A shows a section of the genome-wide Hi-C heatmap for data mapped to the LACHESIS assembly at a resolution of individual CHO-K1 SV raw scaffolds. Only cis interactions are plotted and the smallest LACHESIS groups 7, 8 and 9 are not included because of visual clarity.

FIG. 2B shows a 100% stacked bar chart displaying the average percentage of close cis (<10 kb), far cis (>10 kb) and trans unique, valid di-tags across CHO-K1 SV 10E9 Hi-C replicates mapped to individual input CHO-K1 SV scaffolds and the final LACHESIS assembly. For comparison, distributions of close cis, far cis and trans di-tags, averaged across replicates of equivalent Hi-C datasets derived from human embryonic stem cells and mouse fetal liver cells are included (Nagano, T. et al. Comparison of Hi-C results using in-solution versus in-nucleus ligation. Genome Biol. 16, 175 (2015)).

FIG. 3A shows the structural characteristics for candidate HI loci SEQ ID NO: 3 (location indicated by the diamond). Results of Hi-C PCA illustrating that the candidate locus resides within an active euchromatic-like region (left). Location of candidate locus with respect to TADs identified in the vicinity (middle). Interaction profile of the candidate locus HindIII restriction fragment annotated with ATAC-Seq, H3K4me3, H3K27ac and H3K4me1 signal and the locations of baited, promoter HindIII restriction fragments (right).

FIG. 3B shows the structural characteristics for candidate HI loci SEQ ID NO: 2 (location indicated by the diamond). Results of Hi-C PCA illustrating that the candidate locus resides within an active euchromatic-like region (left). Location of candidate locus with respect to TADs identified in the vicinity (middle). Interaction profile of the candidate locus HindIII restriction fragment annotated with ATAC-Seq, H3K4me3, H3K27ac and H3K4me1 signal and the locations of baited, promoter HindIII restriction fragments (right).

FIG. 3C shows the structural characteristics for the current industrially relevant Fer1L4 landing pad (location indicated by the diamond). Results of Hi-C PCA illustrating that the candidate locus resides within an active euchromatic-like region (left). Location of candidate locus with respect to TADs identified in the vicinity (middle). Interaction profile of the candidate locus HindIII restriction fragment annotated with ATAC-Seq, H3K4me3, H3K27ac and H3K4me1 signal and the locations of baited, promoter HindIII restriction fragments (right).

FIG. 4A-FIG. 4D show the result of screening a subset of genomic loci taken from Table 1 for expression of an integrated eGFP reporter cassette under the control of a CMV promoter. The candidate loci were identified by the screening process described in FIG. 1 and were empirically tested by targeting to the loci an identical CMV-eGFP expression cassette using the Cas9 nuclease in combination with loci-specific guide RNAs. The CMV-eGFP cassette was transfected into cells contained within the donor plasmid shown in FIG. 4A, which also expressed the ‘pseudo gRNA’ sequence required for in vivo Cas9-mediated cleavage of the CMV-eGFP cassette from the plasmid after transfection. Once released from the plasmid the CMV-eGFP cassette is targeted for integration to the required genomic locus by expression of the locus-specific gRNA, cloned into the donor plasmid upstream of the gRNA scaffold sequence at the Bbsl sites. The Cas9 nuclease was supplied at co-transfection on a separate plasmid (not shown). FIG. 4B shows the percentage of GFP positive cells achieved in pools of the Chinese Hamster Ovary SSI 10E9 cell line (Zhang et al., Biotechnol Prog. 2015: 31(6) 1645-56), thirteen days following transfection with both the Cas9 and CMV-eGFP donor plasmids, with the median GFP signal of the GFP+ cells for each pool shown in FIG. 4C. In FIG. 4C the two bars for each target loci represent technical replicates of the flow cytometer analysis. To confirm on-target integration of the CMV-eGFP cassette in each pool, a PCR-based assay was used on extracted genomic DNA (FIG. 4D). A PCR product is only produced upon on-target genome integration, with no PCR product being produced when the donor plasmid only (‘D’) is used as the template. ‘Donor’ refers to the donor plasmid, ‘Het Control’ refers to the heterochromatin control integration site, with ‘Fer1I4’ referring to the landing pad with the 10E9 cell line referred to below.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to the construction of 3D maps of a cell genome, and in one particular embodiment to the construction of 3D maps of the Chinese Hamster Ovary cell genome. Also disclosed is the use of such maps to identify high performance integration sites (HI loci) from which recombinant transgenes can be expressed. The 3D maps can be generated in one particular embodiment described further herein by use of a combination of orthogonal methods such as ATAC-seq (Assay for Transposase-Accessible Chromatin using sequencing) (Buenrostro et al. 10:1213-8 (2013)), Hi-C, and Promoter Capture Hi-C combined with RNA-Seq data on genome-wide transcriptional activity as well as datasets of the methylation and acetylation of the nuclear histones. Through such approaches, a global picture can be generated of the 3D genome as well as its expression profile, which can inform the recognition and design of H1 loci.

According to one embodiment, disclosed is a mammalian cell that includes an RTS integrated within an HI locus. Also disclosed are rP producer cell lines incorporating the mammalian cells and methods for forming such mammalian cells. HI loci described herein and methods for identifying HI loci in cell genomes have been developed through understanding and mapping of the 3D hierarchical structure of chromatin in mammalian cells. HI loci are present in transcriptionally active environments that can provide both chromatin accessibility and epigenetic stability. As such, SSI mammalian cells incorporating RTS at one or more HI loci (i.e., completely within, overlapping, or +/− about 5 Kb) can provide predictable and stable transgene production. For instance, expression of a GOI in a mammalian cell as disclosed can be stable over about 70, about 100, about 150, about 200, or about 300 generations. As utilized herein, expression can be considered “stable” if it decreases by about 30% or less, or is maintained at the same level or at an increased level over time (e.g., about 30% or more) as compared to the initial expression level immediately following production initiation. In some embodiments, expression is considered stable if volumetric productivity changes by less than ±30% or is maintained at the same level. In some embodiments, an SSI host cell can produce about 1.5 g/L, about 2 g/L, about 3 g/L, about 4 g/L, or about 5 g/L or more of an expression product of a GOI. In some embodiments, SSI cells (e.g., SSI cell lines) can be maintained in culture without further selection. As such, disclosed cell lines can be more acceptable to regulatory agencies.

As used herein, the term “about” is used to indicate that a value includes the inherent variation of error for the method/device being employed to determine the value, or the variation that exists among the study subjects. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

In one embodiment, mammalian cells can be derived from Chinese Hamster Ovary (CHO) cells. While much of this discussion refers to CHO cells and cell lines, it should be understood however that this disclosure is in no way limited to any particular cell type and as referred to herein, the term “mammalian cell” includes cells from any member of the order Mammalia. Mammalian cells encompassed herein can include, without limitation, human cells, mouse cells, rat cells, monkey cells, hamster cells, bovine cells, and the like. In some embodiments, the mammalian cell is a mouse cell (e.g. mouse myeloma such as NSO or SP2/0 cell lines), a human cell, a Chinese hamster ovary (CHO) cell, a CHO-K1 cell, a CHO-DXB11 cell, a CHO-DG44 cell, a CHOK1SV™ cell including all variants (e.g. CHOK1SV™ POTELLIGENT®, Lonza, Slough, UK), a CHO glutamine synthetase knockout cell including all variants (e.g., GS-KO™, Xceed™), a DG44 CHO cell, a DUXB11 CHO cell, a CHOS, a CHO FUT8 GS knock-out cell, a CHOZN, or any CHO-derived cell.

According to one embodiment, HI loci that are naturally present within a genome can be identified, and using this identification, mammalian cells can be developed that incorporate heterologous nucleic acid molecules chromosomally-integrated at one or more of the HI loci. For example, heterologous nucleic acid molecules can encompass an exogenous cassette designed to express a GOI in formation of cell lines for production of recombinant proteins.

As used herein, the terms “nucleic acid,” “nucleic acid molecule,” and “oligonucleotide” are interchangeable and refer to a polymeric compound comprising covalently linked nucleotides. The terms include poly (ribonucleic acid) (RNA) and poly (deoxyribonucleic acid) (DNA), both of which may be single- or double-stranded. DNA includes, but is not limited to, complimentary DNA (cDNA), genomic DNA, plasmid or vector DNA, and synthetic DNA. RNA includes, but is not limited to, mRNA, tRNA, rRNA, snRNA, microRNA, miRNA, or MIRNA.

As used herein, the terms “peptide,” “polypeptide,” and “protein” are interchangeable and refer to a polymeric form of amino acids of any length, which can include coded and non-coded amino acids, chemically or biochemically modified or derivatized amino acids, and polypeptides having modified peptide backbones. The term “chain” and polypeptide “chain” are used interchangeably herein and refer to a polymeric form of amino acids of a single peptide backbone. The term “amino acid” refers to both natural and unnatural, i.e., synthetic, amino acids.

As used herein, the term “recombinant” when used in reference to a nucleic acid molecule, peptide, polypeptide, or protein means of, or resulting from, a new combination of genetic material that is not known to exist in nature. A recombinant molecule can be produced by any of the well-known techniques available in the field of recombinant technology, including, but not limited to, polymerase chain reaction (PCR), gene cutting (e.g., using restriction endonucleases), DNA ligation (e.g., using a DNA ligase enzyme), RI, RMCE, CRISPR-mediated technologies, solid state synthesis of nucleic acid molecules, peptides, or proteins, as well as combinations of techniques. In some embodiments, “recombinant” refers to a viral vector or virus that is not known to exist in nature, e.g. a viral vector or virus that has one or more mutations, nucleic acid insertions, or heterologous genes in the viral vector or virus. In some embodiments, “recombinant” refers to a cell or host cell that is not known to exist in nature, e.g. a cell or host cell that has one or more mutations, nucleic acid insertions, or heterologous genes in the cell or host cell.

As used herein, the term “gene” refers to an assembly of nucleotides that encode a polypeptide and includes cDNA and genomic DNA nucleic acid molecules. “Gene” also refers to a nucleic acid fragment that can act as a regulatory element preceding (5′ non-coding sequences) and following (3′ non-coding sequences) a coding sequence. Heterologous genes can be integrated in a host cell genome with a single copy, with multiple copies and/or at predefined copy numbers.

As used herein, the term “regulatory element” refers to a genetic element which controls some aspect of the expression of nucleic acid sequences.

As used herein, the terms “promoter,” “promoter sequence,” or “promoter region” are interchangeable and refer to a DNA regulatory region/sequence capable of binding RNA polymerase and involved in initiating transcription of a downstream coding or non-coding sequence. In some examples of the present disclosure, the promoter sequence includes the transcription initiation site (also referred to herein as a transcription start site (TSS)) and extends upstream to include the minimum number of elements necessary to initiate transcription at levels detectable above background. In some embodiments, the promoter sequence includes a TSS, as well as protein binding domains responsible for the binding of RNA polymerase. Eukaryotic promoters will often, but not always, contain “TATA” boxes and “CAT” boxes. Various promoters, including inducible promoters, leaky promoters, synthetic promoters, etc. may be used to drive gene expression in host cells and/or vectors of the present disclosure.

As used herein, the term “heterologous” refers to a nucleic acid sequence, e.g., a promoter optionally operably linked to a GOI, that is derived from a different species than the host cell in which it is located or is that derived from the same species, but is naturally found in a different location in the species (or host cell). A heterologous nucleic acid sequence can be derived from a prokaryotic system or a eukaryotic system. A coding or non-coding sequence that is associated with a heterologous regulatory sequence (e.g., that is downstream of and transcribed through initiation of a heterologous promoter) can be either endogenous to the heterologous regulatory sequence (e.g., a heterologous promoter is operably linked to the sequence in the natural setting) or can be heterologous to the heterologous regulatory sequence (e.g., a heterologous promoter is not operably linked to the sequence in the natural setting).

As used herein, the term “endogenous” refers to a nucleic acid sequence that is naturally present in the host cell. For instance, an endogenous promoter can be operably linked to initiate transcription of a downstream coding or noncoding sequence that is heterologous to the host cell.

As used herein, the terms “in operable combination,” “in operable order,” and “operably linked” are interchangeable and refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For instance, a GOI, an ancillary gene, a recombinase-encoding gene, or a non-coding sequence can be operably linked to a promoter, and the nucleic acid sequence can be chromosomally-integrated into the host cell.

As referred to herein, the term “chromosomally-integrated” or “chromosomal integration” refers to the stable incorporation of a nucleic acid sequence into the chromosome of a host cell, e.g. a mammalian cell. i.e., a nucleic acid sequence that is chromosomally-integrated into the genomic DNA (gDNA) of a host cell, e.g. a mammalian cell.

As used herein, the terms “chromosomal locus” and “locus” (pl. “loci”) are used interchangeably and refer to a defined location of nucleic acids on the chromosome of a cell. In some embodiments, a locus may comprise at least one gene. By way of example, a chromosomal locus can include about 500 base pairs to about 100,000 base pairs; about 5,000 base pairs to about 75,000 base pairs; about 5,000 base pairs to about 60,000 base pairs; about 20,000 base pairs to about 50,000 base pairs; about 30,000 base pairs to about 50,000 base pairs; or about 45,000 base pairs to about 49,000 base pairs. In some embodiments, a chromosomal locus can extend up to about 100 base pairs, about 250 base pairs; about 500 base pairs; about 750 base pairs; about 1000 base pairs; or about 5000 base pairs to the 5′ and/or the 3′ end of a defined nucleic acid sequence.

In one embodiment, a method can include identifying HI loci in a genome. HI loci can be within an active genome compartment of accessible chromatin and can be within about 30,000 base pairs in either the 5′ or the 3′ direction of a topologically associated domain boundary. In one embodiment, the first set of peaks can be within active genomic compartments (for instance as defined by Principle Component Analysis Methods (PCA)) and can also be within open chromatin (for instance as defined by ATAC-seq), but this is not a requirement of a method, and in other embodiments, the first set of peaks can include those peaks that are within active genomic compartments within the whole of the mapped accessible chromatin. HI loci can also overlap a region that interacts with at least one enhancer element. Accordingly, identification of HI loci can include 3D mapping of a genome to identify a set of peaks that meet these criteria.

As used herein, the term “topologically associated domain,” and “TAD,” and “contact domain” are used interchangeably and refer to highly conserved genomic regions that contain nucleic acid sequences that preferentially physically interact with one another. As such, nucleic acid sequences within a TAD will physically interact with one another more frequently than with sequences that exist external to the confines of the TAD. A TAD can extend from thousands to millions of base pairs. A TAD can be partitioned by a boundary region (a “TAD boundary”), that can be enriched in factors associated with active transcription. For instance, a TAD boundary region can exhibit a relatively high level of CTCF binding. A TAD boundary region can also be recognized by the presence of a relatively large numbers of tRNA genes and housekeeping genes (e.g., actin, GAPDH, ubiquitin, etc.).

As used herein, the terms, “enhancer,” “enhancer element,” “putative active enhancer element,” and “predicted active enhancer element” are used interchangeably and refer to a DNA regulatory region/sequence capable of increasing the transcription rate of a target gene and that does not overlap with regions 2 Kb upstream or 2 Kb downstream of an annotated transcription start site but is, as indicated by ChromHMM analysis (see e.g., Ernst and Kellis M. Nat Protoc. 12:2478-2492 (2017)), enriched for an ATAC-Seq signal (indicating open, accessible chromatin), and H3K4me1 and H3K27ac histone marks (Shlyueva et al. 2014. Nat Rev Genet. 15:272-86).

The term “enhancer element” can also encompass an “interacting putative active enhancer restriction fragment” which refers to a HindIII restriction fragment that does not itself contain an annotated transcription start site (TSS) and/or overlaps a genomic region enriched for either H3K27me3 or H3K9me3 histone marks (as indicated by ChromHMM analysis), but does overlap a putative active enhancer (as defined above) and does interact in cis and in multiple PCHi-C (Promoter Capture Hi-C) replicates, with a HindIII restriction fragment containing an annotated TSS.

An enhancer element can be linked to a promoter for a coding or non-coding sequence and can be located either upstream or downstream of a promoter and associated gene. An enhancer element can often exhibit activity when placed in either orientation, and enhancers may be active when located at considerable distances from a promoter. For instance, an enhancer element can be located up to about 1,000,000 either upstream or downstream of a TSS and can be contiguous or non-contiguous with a TSS. Methods for detecting enhancer activity are known in the art, for e.g., see Molecular Cloning, A Laboratory Manual, Second Edition, (Sambrook Fritsch, Maniatis, Eds., Cold Spring Harbor Laboratory Press, Cold Spring Harbor N.Y., 1989). The activity associated with such enhancer elements—first described for viral sequences (Banerji et al., 1981, Moreau et al., 1981) and subsequently for sequences originating from metazoan gene loci (Banerji et al., 1983, Gillies et al., 1983)—includes the activation of transcription regardless of the element's location or orientation relative to the promoter within a plasmid construct.

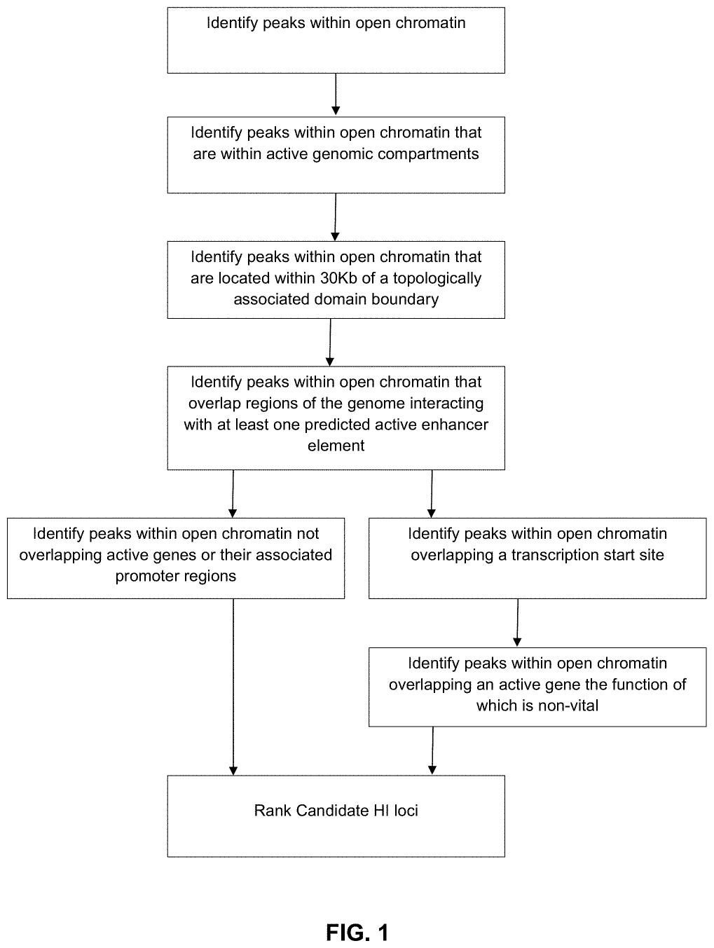

As illustrated in FIG. 1, a method can include identification of peaks within accessible chromatin. As used herein, the term “peak” refers to a region of the genome that includes an increase in the number of DNA sequencing reads (i.e. sequencing read depth). For example, an increase in the sequencing read depth above a normalized background model for a genomic region as revealed by ATAC-Seq can indicate open chromatin, whereas an increase above a set threshold (e.g. normalised CHiCAGO score of 5 or above; Cairns J, et al., Genome Biology. 2016. 17:127) in the number of sequencing reads between two HindIII restriction fragments from a PCHi-C experiment would indicate a statistically significant cis interaction between two genomic regions. The term “peak” can also refer to an increase above a predetermined threshold in the contact frequency between two points in the genome as revealed by techniques such as Hi-C and PCHi-C.

In some embodiments, peak identification can be carried out as a consequence of performing a sequence protocol, e.g., a ChIP-sequencing or MeDIP-seq (Methylated DNA immunoprecipitation sequencing) protocol. Any peak calling tools as are known in the art may be utilized in identifying peaks as defined herein. Many of the known peak calling tools are optimized for only some kind of assays such as only for transcription-factor ChIP-seq or only for DNase-seq. However peak identification methodologies encompassed herein are not limited to such tools and any peak calling methods and software including, without limitation, DFilter, GEM, MACS2 (Zhang et al. Model-based Analysis of ChIP-Seq (MACS). Genome Biol (2008) vol. 9 (9) pp. R137), MUSIC, BCP, Threshold-based Method™ and ZINBA can be utilized. Peak calling methods can include methods based on generalized optimal theory of detection as well as those capable of utilization with different types of sequencing data.

Data sets selected for mapping and identification of peaks in a sequence of interest can be optimized depending upon the type of peaks being identified. Moreover, peaks can be identified through utilization of multiple data sets as reference sequences. For instance, peaks can be identified through utilization of simulated ChIP-seq data sets, real data sets, combinations thereof and in conjunction with mathematical analyses (e.g., utilization of a Poisson test to rank candidate peaks). Data sets can include, without limitation, ChIP-seq, ATAC-seq (see e.g., US Patent Application Publication No. 2016/0060691 to Giresi, et al.; Buenrostro, et al. 2015 “ATAC-Seq: A method for assaying chromatin accessibility genome-wide.” Curr Protoc Mol Bio 109: 21.29.1-21.29.9), Hi-C, Promoter Capture Hi-C (PCHi-C) (see e.g., US Patent Application Publication No. 2016/0194713 to Fraser, et al.), RNA-seq, and any combination thereof. Other datasets as are known in the art can be utilized e.g., Feichtinger ChiP-Seq datasets (Accession Number—PRJEB9291) (see e.g., Feichtinger et al. Biotechnol Bioeng. 113(10):2241-53 (2016)). In some embodiment a plurality of data sets (e.g., a plurality of Hi-C data sets) can be utilized to assemble chromosome-scale de novo reference genomic data that can be utilized in identification of HI loci in a sequence of interest using, for example SALSA or LACHESIS software (see e.g., Burton, et al., 2013 “Chromosome-scale scaffolding of de novo genome assemblies based on chromatin interactions.” Nat Biotechnol 31:1119-1125).

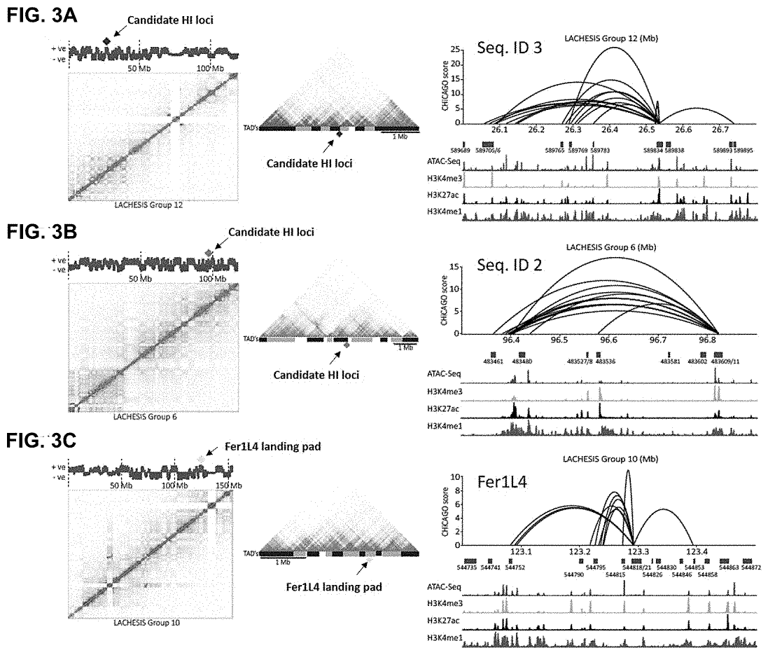

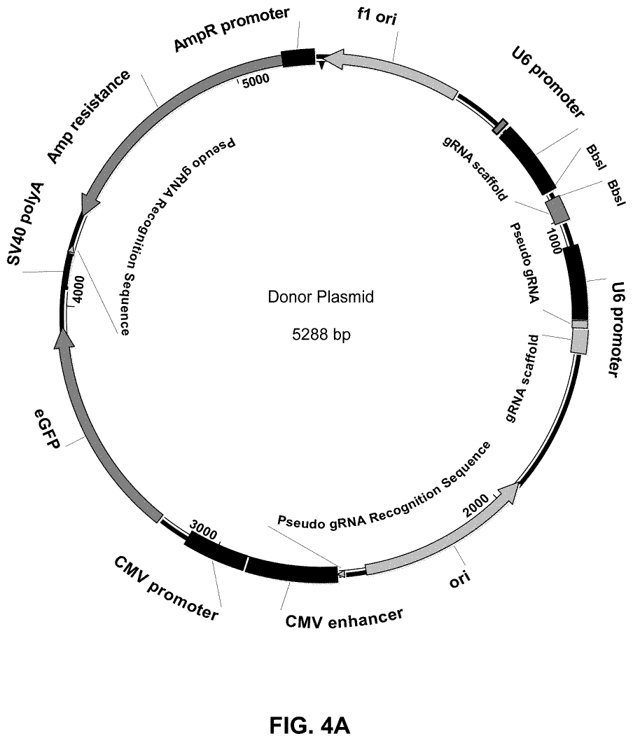

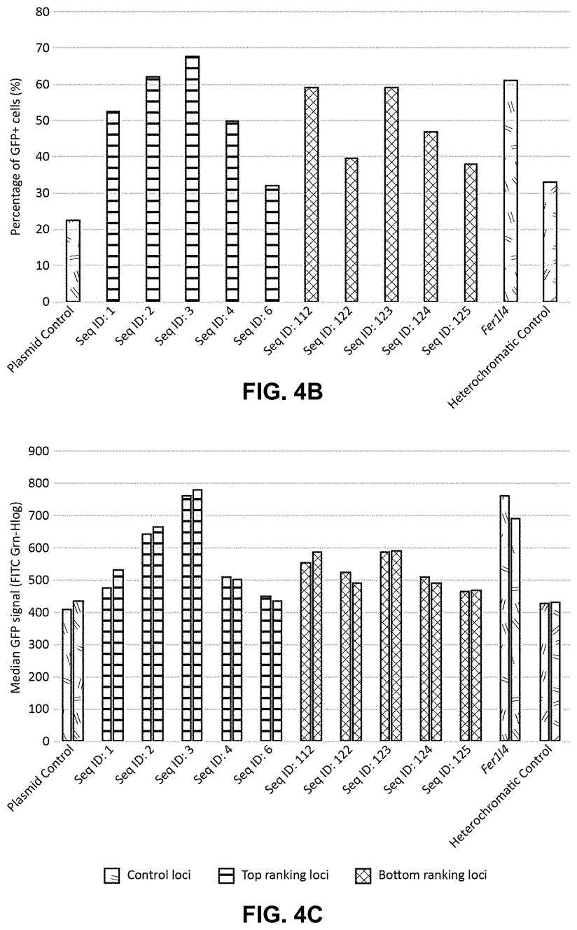

As illustrated in FIG. 1, HI loci can be within an active genomic compartment of accessible chromatin (also FIG. 3). Thus, identification of HI loci on a genome can include initial identification of peaks in accessible chromatin (for instance through utilization of a peak calling algorithm utilizing ATAC-seq) followed by analysis to determine which of those peaks are present in active genomic compartments as indicated in FIG. 1. It should be understood, that the specific order of identification steps illustrated in FIG. 1 are representative only, and the disclosed methods are not limited to any particular order by which the various aspects of the genome are mapped. For instance, in the embodiment illustrated in FIG. 1, the step of identifying all peaks within accessible chromatin that are within active genomic compartments is carried out prior to identification of peaks located within 30 Kb of a TAD, but the particular order of these and other steps in the embodiment can be modified.

According to one embodiment, identification of peaks of accessible chromatin found within active genomic compartments of a sequence of interest can be carried out by comparison of the genomic sequence of interest with a reference sequence. A reference sequence can be a single known sequence or can be assembled through a compilation of known sequences (e.g., through utilization of LACHESIS software with a plurality of Hi-C and/or PCHi-C data sets). In one embodiment, the reference sequence can be examined to identify all peaks of interest, e.g., all ATAC-Seq peaks of the reference sequence. Comparison between peaks found in accessible chromatin with those found in active genomic compartments can provide a set of peaks that are present in active genomic compartments of the accessible chromatin of the reference sequence. Upon mapping the sequence of interest against the reference sequence, a filtering protocol can be carried out to identify the peaks in the sequence of interest that are in accessible chromatin and within active genomic compartments.

HI loci can also be within about 30,000 base pairs of a TAD boundary region. Accordingly, in one embodiment as illustrated in FIG. 1, following identification of a set of peaks in the sequence of interest that are present in active genomic compartments of accessible chromatin, this set of peaks can be further analyzed to determine which of those peaks are also within about 30,000 base pairs (either upstream or downstream) of a TAD boundary region. This can be carried out through mapping the sequence of interest against the same or a different reference sequence. If necessary, the TAD boundary regions can be identified in the reference sequence prior to the mapping. In one embodiment, TAD boundary regions can be identified according to methods described using a “directionality index” (see e.g., in Dixon et al., 2012, “Topological domains in mammalian genomes identified by analysis of chromatin interactions.” Nature. 485(7398):376-80). Of course, other methods and tools for identifying TAD boundary regions can likewise be utilized.

In one embodiment (described further in the examples section below), identification of active genomic compartments and TAD boundary locations can be carried out by comparing a reference sequence (e.g., a genome assembly, one or a compilation of Hi-C data sets, etc.) to the sequence of interest, for instance by applying an algorithm to a genomic assembly obtained by use of LACHESIS software mapped to the sequence of interest. Upon identification of the TAD boundaries and through utilization of one or more reference genomic sequences that are complete over at least the active genomic compartments of accessible chromatin sections of the genome, peaks within about 30,000 base pairs of each TAD boundary can be identified.

As shown in the embodiment illustrated in FIG. 1, the set of peaks identified as being within about 30,000 base pairs of a TAD boundary and also within an active genomic compartment of accessible chromatin can be further examined to determine which of those peaks also overlap regions of the genome that interact with at least one enhancer element (generally cis interactions though trans interactions are also encompassed herein). For example, a method can include identification of regions of a genome that interact with at least one enhancer element using data sets such as, and without limitation to, PCHi-C, ATAC-Seq, ChIP-seq, ChromHMM, or combinations thereof. In one embodiment, statistically significant enhancer interaction predictions can be identified by PCHi-C and ChromHMM analysis of the reference sequence mapped against the sequence of interest. The peaks previously identified in the sequence of interest can then be further filtered to include only those that interact with an enhancer element. This further filtering can narrow the set of peaks to those falling within these regions. The resulting set of filtered peaks can be used to identify HI loci of the genome, i.e., each of these peaks can define a potential HI locus of the genome.

Further refinement of the HI loci can be carried out depending upon the type of promoter that is intended to be used in driving transcription of a heterologous gene to be inserted into the genome.

HI loci in those embodiments in which a heterologous promoter is to be used in transcription of a GOI can preferably not overlap any genes of the genome. In one embodiment, the HI loci can include those loci that do not overlap any active genes of the genome, but embodiments that incorporate a heterologous promoter are not limited to lack of overlap with active genes. In one embodiment, the HI loci will not overlap any promoter of any genes, or any promoter of any active genes of the genome in one embodiment. In one embodiment, the HI loci will not fall within about 1000 base pairs on either side of any such promoter. Thus, in one embodiment a method can further include filtering of the potential HI loci previously obtained through remapping a reference sequence to the sequence of interest to identify peaks external to these regions (e.g., active genes and their associated promoter regions (± about 1000 base pairs of the promoter)) of the sequence of interest. These peaks can then be identified as desirable HI loci.

HI loci for use in those embodiments in which an in situ endogenous promoter is to be used in transcription of a GOI can overlap the in situ endogenous TSS for an active gene the expression or lack of expression of which is non-vital to the cell, i.e., the recombinant cell can survive absent the active gene. Thus, as shown in the flow path on the right side of FIG. 1, a method can further include filtering the potential HI loci previously obtained through remapping of a reference sequence to the sequence of interest to identify the non-vital active genes and their associated TSS within the active compartments of the accessible chromatin. The genes of interest can also be examined for other characteristics that may affect the use of the gene's promoter in expression of an inserted RTS, e.g., lethality for example. Those peaks that overlap these regions of suitable genes can then be identified as desirable HI loci.

The resulting set of peaks that fit into all of the desired categories for a particular application can provide HI loci of the genome. For instance, HI loci for use in applications encompassing utilization of a heterologous promoter can include peaks located in active genomic compartments of accessible chromatin and within about 30,000 base pairs (upstream or downstream) of a TAD boundary. In addition, these HI loci can overlap regions of the genome that interact with an enhancer element and will generally not overlap genes or their associated promoter regions.

HI loci for use in applications encompassing utilization of an in situ endogenous promoter can also encompass peaks located in active genomic compartments of accessible chromatin and within about 30,000 base pairs (upstream or downstream) of a TAD boundary and these HI loci can also overlap regions of the genome that interact with an enhancer element. In addition, these HI loci will overlap endogenous TSS of an active gene that is confined within an active genomic compartment of accessible chromatin and that has a function that has been classified as non-vital to the cell.

In one embodiment, a method can include ranking the HI loci following identification thereof. For instance, HI loci can be ranked based upon one or more of the expression level of one or more genes associated with a locus, the distance from the locus to the nearest TAD boundary, the number of predicted enhancer interactions, and the steady state mRNA levels of one or more genes associated with the locus. For example, in one embodiment, each identified HI locus can be ranked according to only a single parameter, and these multiple rankings for all HI loci can then be analyzed to determine an overall ranking. The combinatorial analysis can be weighted or not, as desired. For example, a simple additive score for each ranking of each locus can be utilized to determine an overall ranking according to a non-weighted combinatorial method. High ranking loci, e.g., those associated with a high expressing gene, close to the nearest TAD boundary, and predicted to have a large number of enhancer interactions can be highly desirable loci for insertion of an RTS.

Through utilization of the described methods, HI loci can be identified in any mammalian cell. By way of example, Table 1, below, provides examples of CHO genomic HI loci identified according to the disclosed methods. However, it should be understood that CHO genomic HI loci are in no way limited to the loci of Table 1 and homologous sequences to any one of SEQ ID NO: 1-125 are encompassed herein. In other embodiments, CHO genomic HI loci can be within about 5000 base pairs, about 1000 base pairs, about 750 base pairs, about 500 base pairs, about 250 base pairs, or about 100 base pairs to the 5′ and/or the 3′ end of a locus as identified in Table 1 below.

An HI locus can have a small number of mismatches or gaps as compared to the sequences of Table 1. For instance, CHO genomic HI loci encompassed herein can have about 10 or fewer mismatches with the sequences described below. For instance, CHO HI loci encompassed herein can have 10, 9, 8, 7, 6, 5, 4, 3, 2, or 1 mismatch with a sequence as described in Table 1 and/or can have 5 or fewer gaps as compared to a sequence as described in Table 1.

HI loci as defined herein can also encompass portions of any one of SEQ ID NO: 1-125 and are not limited to the full-length sequences of SEQ ID NO: 1-125. For instance, HI loci can encompass genomic sequences that are equivalent sequences or homologous sequences to only a portion of any one of SEQ ID NO: 1-125, e.g., equivalent or homologous to a region of from about 5 bp to about 98% or less of any one of SEQ ID NO: 1-125. By way of example, and HI loci encompassed herein can include sequences that are equivalent or homologous to from about 5 bp to about 95%, 90%, 85%, 80%, 80%, 75%, 70%, 65%, 60%, 55%, 50%, 45%, 40%, 35%, 30%, 25%, 20%, 15%, 10% or 5%, of the total length of any one of SEQ ID NO: 1-125.

As utilized herein, the term “homologue” or “homologous sequences” refers to nucleotide sequences that have sequence homology to the specifically given comparative sequence, e. g. to any one of SEQ ID NO: 1-125 of Table 1 or to a portion of any one of SEQ ID NO: 1-125. As used herein, the term “sequence homology” refers to a measure of the degree of identity or similarity of two sequences based upon an alignment of the sequences which maximizes similarity between aligned nucleotides, and which is a function of the number of identical nucleotides, the number of total nucleotides, and the presence and length of gaps in the sequence alignment. A variety of algorithms and computer programs are available for determining sequence similarity using standard parameters. In one embodiment, sequence homology can be measured using the BLASTn program for nucleic acid sequences, which is available through the National Center for Biotechnology Information (www.ncbi.nlm.nih.gov/), and is described in, for example, Altschul et al. (1990), J Mol. Biol. 215:403-410; Gish and States (1993), Nature Genet. 3:266-272; Madden et al. (1996), Meth. Enzymol. 266: 131-141; Altschul et al. (1997), Nu-cleic Acids Res. 25:33 89-3402); Zhang et al. (2000), J. Comput. Biol. 7(I-2):203-14. In one embodiment, sequence homology of two nucleotide sequences can be determined by the score based upon the following parameters for the BLASTn algorithm: word size=1 1; gap opening penalty=−5; gap extension penalty=−2; match reward=1; and mismatch penalty=−3.

Sequences of Table 1 below are referenced to the publically available BGI CHO database as well as to the publically available GenBank® at NCBI genetic sequence database. The GenBank assembly accession number for the sequences of Table 1 is GCA_000223135.1 and the BGI CHO RefSeq assembly accession number for the sequence of Table 1 is GCF_000223135.1 submitted by the Beijing Genomics Institute Aug. 23, 2011. The “start” and “end” numbers referred to in Table 1 refer to the starting and ending nucleotides of each HI loci within the publicly available complete sequences.

TABLE 1

| Identifier | Ref Seq ID (BGI) | Gen Bank ID | Start | End |
|---|---|---|---|---|
| SEQ ID | NW_003613881.1 | JH000302.1 | 84716 | 85287 |
| NO: 1 | | | | |
| SEQ ID | NW_003615210.1 | JH001631.1 | 71701 | 72159 |
| NO: 2 | | | | |
| SEQ ID | NW_003614717.1 | JH001138.1 | 337655 | 338272 |
| NO: 3 | | | | |
| SEQ ID | NW_003613581.1 | JH000002.1 | 7425820 | 7426374 |
| NO: 4 | | | | |
| SEQ ID | NW_003613631.1 | JH000052.1 | 433858 | 434333 |
| NO: 5 | | | | |
| SEQ ID | NW_003613632.1 | JH000053.1 | 89869 | 91002 |
| NO: 6 | | | | |
| SEQ ID | NW_003614893.1 | JH001314.1 | 289309 | 290428 |
| NO: 7 | | | | |
| SEQ ID | NW_003613806.1 | JH000227.1 | 1874102 | 1874527 |
| NO: 8 | | | | |
| SEQ ID | NW_003613889.1 | JH000310.1 | 1023321 | 1023703 |
| NO: 9 | | | | |
| SEQ ID | NW_003614194.1 | JH000615.1 | 669964 | 671525 |
| NO: 10 | | | | |
| SEQ ID | NW_003613902.1 | JH000323.1 | 546881 | 549199 |
| NO: 11 | | | | |
| SEQ ID | NW_003613993.1 | JH000414.1 | 602605 | 604105 |
| NO: 12 | | | | |
| SEQ ID | NW_003615210.1 | JH001631.1 | 82695 | 83356 |
| NO: 13 | | | | |
| SEQ ID | NW_003614722.1 | JH001143.1 | 438868 | 439552 |
| NO: 14 | | | | |
| SEQ ID | NW_003615490.1 | JH001911.1 | 245039 | 245947 |
| NO: 15 | | | | |
| SEQ ID | NW_003615002.1 | JH001423.1 | 367133 | 368502 |
| NO: 16 | | | | |
| SEQ ID | NW_003613898.1 | JH000319.1 | 674034 | 676716 |
| NO: 17 | | | | |
| SEQ ID | NW_003614180.1 | JH000601.1 | 918619 | 919880 |
| NO: 18 | | | | |
| SEQ ID | NW_003613840.1 | JH000261.1 | 1252935 | 1253947 |
| NO: 19 | | | | |
| SEQ ID | NW_003614393.1 | JH000814.1 | 273894 | 274567 |
| NO: 20 | | | | |
| SEQ ID | NW_003613988.1 | JH000409.1 | 639654 | 640125 |
| NO: 21 | | | | |
| SEQ ID | NW_003613993.1 | JH000414.1 | 601804 | 602221 |
| NO: 22 | | | | |
| SEQ ID | NW_003614079.1 | JH000500.1 | 660401 | 661311 |
| NO: 23 | | | | |
| SEQ ID | NW_003613671.1 | JH000092.1 | 1123441 | 1123859 |
| NO: 24 | | | | |
| SEQ ID | NW_003616472.1 | JH002893.1 | 4334 | 4713 |
| NO: 25 | | | | |
| SEQ ID | NW_003614638.1 | JH001059.1 | 170761 | 171294 |
| NO: 26 | | | | |
| SEQ ID | NW_003613665.1 | JH000086.1 | 924921 | 925461 |
| NO: 27 | | | | |
| SEQ ID | NW_003614172.1 | JH000593.1 | 309722 | 310518 |
| NO: 28 | | | | |
| SEQ ID | NW_003613597.1 | JH000018.1 | 1400069 | 1400754 |
| NO: 29 | | | | |
| SEQ ID | NW_003614184.1 | JH000605.1 | 1035750 | 1036220 |
| NO: 30 | | | | |
| SEQ ID | NW_003613594.1 | JH000015.1 | 2116973 | 2117455 |
| NO: 31 | | | | |
| SEQ ID | NW_003616575.1 | JH002996.1 | 82707 | 83088 |
| NO: 32 | | | | |
| SEQ ID | NW_003613642.1 | JH000063.1 | 220728 | 221685 |
| NO: 33 | | | | |
| SEQ ID | NW_003614184.1 | JH000605.1 | 1034965 | 1035473 |
| NO: 34 | | | | |
| SEQ ID | NW_003613706.1 | JH000127.1 | 2567082 | 2567544 |
| NO: 35 | | | | |
| SEQ ID | NW_003616080.1 | JH002501.1 | 73799 | 74274 |
| NO: 36 | | | | |
| SEQ ID | NW_003614048.1 | JH000469.1 | 762152 | 763323 |
| NO: 37 | | | | |
| SEQ ID | NW_003614624.1 | JH001045.1 | 33306 | 34526 |
| NO: 38 | | | | |
| SEQ ID | NW_003613597.1 | JH000018.1 | 1398585 | 1399064 |
| NO: 39 | | | | |
| SEQ ID | NW_003615594.1 | JH002015.1 | 100451 | 101631 |
| NO: 40 | | | | |
| SEQ ID | NW_003613921.1 | JH000342.1 | 1256190 | 1256908 |
| NO: 41 | | | | |
| SEQ ID | NW_003613580.1 | JH000001.1 | 4564076 | 4564939 |
| NO: 42 | | | | |
| SEQ ID | NW_003614741.1 | JH001162.1 | 284341 | 285116 |
| NO: 43 | | | | |
| SEQ ID | NW_003614645.1 | JH001066.1 | 493683 | 495124 |
| NO: 44 | | | | |
| SEQ ID | NW_003615558.1 | JH001979.1 | 103961 | 104438 |
| NO: 45 | | | | |
| SEQ ID | NW_003615002.1 | JH001423.1 | 360524 | 362561 |
| NO: 46 | | | | |
| SEQ ID | NW_003613613.1 | JH000034.1 | 2819741 | 2820144 |
| NO: 47 | | | | |
| SEQ ID | NW_003614741.1 | JH001162.1 | 284126 | 283210 |
| NO: 48 | | | | |
| SEQ ID | NW_003613880.1 | JH000301.1 | 1108180 | 1108637 |
| NO: 49 | | | | |
| SEQ ID | NW_003614361.1 | JH000782.1 | 23505 | 23965 |
| NO: 50 | | | | |
| SEQ ID | NW_003613703.1 | JH000124.1 | 1843919 | 1844237 |
| NO: 51 | | | | |
| SEQ ID | NW_003616253.1 | JH002674.1 | 5152 | 5993 |
| NO: 52 | | | | |
| SEQ ID | NW_003613870.1 | JH000291.1 | 74116 | 75073 |
| NO: 53 | | | | |
| SEQ ID | NW_003613639.1 | JH000060.1 | 1786158 | 1786619 |
| NO: 54 | | | | |
| SEQ ID | NW_003613601.1 | JH000022.1 | 878305 | 879044 |
| NO: 55 | | | | |
| SEQ ID | NW_003613906.1 | JH000327.1 | 367520 | 368086 |
| NO: 56 | | | | |
| SEQ ID | NW_003614382.1 | JH000803.1 | 131283 | 131629 |
| NO: 57 | | | | |
| SEQ ID | NW_003613624.1 | JH000045.1 | 3291483 | 3292489 |
| NO: 58 | | | | |
| SEQ ID | NW_003613631.1 | JH000052.1 | 3320573 | 3321349 |
| NO: 59 | | | | |
| SEQ ID | NW_003614043.1 | JH000464.1 | 311064 | 311633 |
| NO: 60 | | | | |
| SEQ ID | NW_003614095.1 | JH000516.1 | 697982 | 698384 |
| NO: 61 | | | | |
| SEQ ID | NW_003616080.1 | JH002501.1 | 76108 | 77308 |
| NO: 62 | | | | |
| SEQ ID | NW_003613906.1 | JH000327.1 | 392805 | 393649 |
| NO: 63 | | | | |
| SEQ ID | NW_003614624.1 | JH001045.1 | 27021 | 27408 |
| NO: 64 | | | | |
| SEQ ID | NW_003613788.1 | JH000209.1 | 1574068 | 1574442 |
| NO: 65 | | | | |
| SEQ ID | NW_003614393.1 | JH000814.1 | 146964 | 147415 |
| NO: 66 | | | | |
| SEQ ID | NW_003613880.1 | JH000301.1 | 1105849 | 1106970 |
| NO: 67 | | | | |
| SEQ ID | NW_003613840.1 | JH000261.1 | 1673953 | 1675296 |
| NO: 68 | | | | |
| SEQ ID | NW_003613658.1 | JH000079.1 | 1494473 | 1496419 |
| NO: 69 | | | | |
| SEQ ID | NW_003614393.1 | JH000814.1 | 144373 | 146735 |
| NO: 70 | | | | |
| SEQ ID | NW_003613925.1 | JH000346.1 | 383977 | 385766 |
| NO: 71 | | | | |
| SEQ ID | NW_003613876.1 | JH000297.1 | 420933 | 421434 |
| NO: 72 | | | | |
| SEQ ID | NW_003613733.1 | JH000154.1 | 1468067 | 1468697 |
| NO: 73 | | | | |
| SEQ ID | NW_003613840.1 | JH000261.1 | 1673114 | 1673548 |
| NO: 74 | | | | |
| SEQ ID | NW_003613638.1 | JH000059.1 | 1801002 | 1802255 |
| NO: 75 | | | | |
| SEQ ID | NW_003613893.1 | JH000314.1 | 528065 | 528983 |
| NO: 76 | | | | |
| SEQ ID | NW_003613601.1 | JH000022.1 | 890427 | 890755 |
| NO: 77 | | | | |
| SEQ ID | NW_003613581.1 | JH000002.1 | 4619839 | 4620323 |
| NO: 78 | | | | |
| SEQ ID | NW_003614382.1 | JH000803.1 | 125976 | 127247 |
| NO: 79 | | | | |
| SEQ ID | NW_003613683.1 | JH000104.1 | 2795103 | 2796221 |
| NO: 80 | | | | |
| SEQ ID | NW_003614391.1 | JH000812.1 | 278384 | 278911 |
| NO: 81 | | | | |
| SEQ ID | NW_003614171.1 | JH000592.1 | 636055 | 636974 |
| NO: 82 | | | | |
| SEQ ID | NW_003613774.1 | JH000195.1 | 1385547 | 1386263 |
| NO: 83 | | | | |
| SEQ ID | NW_003613631.1 | JH000052.1 | 3311170 | 3311535 |
| NO: 84 | | | | |
| SEQ ID | NW_003613788.1 | JH000209.1 | 171502 | 171983 |
| NO: 85 | | | | |
| SEQ ID | NW_003614244.1 | JH000665.1 | 524592 | 525320 |
| NO: 86 | | | | |
| SEQ ID | NW_003614497.1 | JH000918.1 | 22628 | 22961 |
| NO: 87 | | | | |
| SEQ ID | NW_003614195.1 | JH000616.1 | 384900 | 387424 |
| NO: 88 | | | | |
| SEQ ID | NW_003615981.1 | JH002402.1 | 161084 | 161596 |
| NO: 89 | | | | |
| SEQ ID | NW_003614079.1 | JH000500.1 | 335366 | 336028 |
| NO: 90 | | | | |
| SEQ ID | NW_003613599.1 | JH000020.1 | 3922137 | 3922464 |
| NO: 91 | | | | |
| SEQ ID | NW_003613671.1 | JH000092.1 | 1105087 | 1105561 |
| NO: 92 | | | | |
| SEQ ID | NW_003614478.1 | JH000899.1 | 197362 | 198521 |
| NO: 93 | | | | |
| SEQ ID | NW_003613588.1 | JH000009.1 | 4406189 | 4406993 |
| NO: 94 | | | | |
| SEQ ID | NW_003613785.1 | JH000206.1 | 828327 | 828997 |
| NO: 95 | | | | |
| SEQ ID | NW_003613943.1 | JH000364.1 | 499742 | 500158 |
| NO: 96 | | | | |
| SEQ ID | NW_003614337.1 | JH000758.1 | 638567 | 639207 |
| NO: 97 | | | | |
| SEQ ID | NW_003614211.1 | JH000632.1 | 977544 | 977951 |
| NO: 98 | | | | |
| SEQ ID | NW_003613639.1 | JH000060.1 | 1804175 | 1805460 |
| NO: 99 | | | | |
| SEQ ID | NW_003615035.1 | JH001456.1 | 366013 | 368530 |
| NO: 100 | | | | |
| SEQ ID | NW_003615368.1 | JH001789.1 | 25527 | 26437 |
| NO: 101 | | | | |
| SEQ ID | NW_003613658.1 | JH000079.1 | 1488993 | 1489287 |
| NO: 102 | | | | |
| SEQ ID | NW_003613671.1 | JH000092.1 | 1100021 | 1100646 |
| NO: 103 | | | | |
| SEQ ID | NW_003613894.1 | JH000315.1 | 543845 | 544367 |
| NO: 104 | | | | |
| SEQ ID | NW_003614229.1 | JH000650.1 | 714046 | 714488 |
| NO: 105 | | | | |
| SEQ ID | NW_003614295.1 | JH000716.1 | 393467 | 394320 |
| NO: 106 | | | | |
| SEQ ID | NW_003615408.1 | JH001829.1 | 15167 | 15683 |
| NO: 107 | | | | |
| SEQ ID | NW_003614949.1 | JH001370.1 | 105417 | 105823 |
| NO: 108 | | | | |
| SEQ ID | NW_003614116.1 | JH000537.1 | 902568 | 903056 |
| NO: 109 | | | | |
| SEQ ID | NW_003615058.1 | JH001479.1 | 134335 | 136140 |
| NO: 110 | | | | |
| SEQ ID | NW_003616670.1 | JH003091.1 | 37625 | 38505 |
| NO: 111 | | | | |
| SEQ ID | NW_003617099.1 | JH003520.1 | 27020 | 27346 |
| NO: 112 | | | | |
| SEQ ID | NW_003614244.1 | JH000665.1 | 517254 | 517839 |
| NO: 113 | | | | |
| SEQ ID | NW_003614184.1 | JH000605.1 | 1012523 | 1013195 |
| NO: 114 | | | | |
| SEQ ID | NW_003613630.1 | JH000051.1 | 2598585 | 2598973 |
| NO: 115 | | | | |
| SEQ ID | NW_003614105.1 | JH000526.1 | 154628 | 155158 |
| NO: 116 | | | | |
| SEQ ID | NW_003613581.1 | JH000002.1 | 7456163 | 7457018 |
| NO: 117 | | | | |
| SEQ ID | NW_003616693.1 | JH003114.1 | 22038 | 23038 |
| NO: 118 | | | | |
| SEQ ID | NW_003613716.1 | JH000137.1 | 196705 | 197107 |
| NO: 119 | | | | |
| SEQ ID | NW_003614511.1 | JH000932.1 | 523637 | 524645 |
| NO: 120 | | | | |
| SEQ ID | NW_003616693.1 | JH003114.1 | 23111 | 23544 |
| NO: 121 | | | | |
| SEQ ID | NW_003615153.1 | JH001574.1 | 213287 | 213975 |
| NO: 122 | | | | |
| SEQ ID | NW_003613581.1 | JH000002.1 | 1242852 | 1243359 |
| NO: 123 | | | | |
| SEQ ID | NW_003614351.1 | JH000772.1 | 306311 | 306745 |
| NO: 124 | | | | |
| SEQ ID | NW_003613614.1 | JH000035.1 | 1386485 | 1387181 |
| NO: 125 | | | | |

According to one embodiment, upon identification of HI loci of a genome, a mammalian cell can be modified to include a landing pad at an HI locus of the genome. For instance, in one embodiment, a particular HI locus can be selected (e.g., by ranking of the identified HI loci) and an RTS can be inserted at that locus in formation of a site-specific integration site (e.g., within or overlapping any one of SEQ ID NOs: 1-125 or within or overlapping about 5,000 base pairs, about 1000 base pairs, about 750 base pairs, about 500 base pairs, about 250 base pairs, or about 100 base pairs of either the 5′ or 3′ end of any one of SEQ ID NOs: 1-125).

In one embodiment, a integration protocol can be carried out to integrate an expression cassette randomly into the genome of a plurality of cells. For example, in one embodiment a random integration protocol can be carried out and an expression cassette carrying a detectable marker can be integrated into the cells. Following, the cells can be examined to determine integration sites of the cassette and a cell that includes the integration site at an HI locus (e.g., a high ranking HI locus in one embodiment) can be selected. That selected cell can then be utilized to establish a landing pad at the HI locus (e.g., within or overlapping any one of SEQ ID NOs: 1-125 or within about 5,000 base pairs, about 1000 base pairs, about 750 base pairs, about 500 base pairs, about 250 base pairs, or about 100 base pairs of either the 5′ or 3′ end of any one of SEQ ID NOs: 1-125).

As referred to herein, the term “landing pad” refers to a nucleic acid sequence comprising an RTS chromosomally-integrated into a host cell. In some embodiments, a landing pad comprises two or more RTS chromosomally-integrated into a host cell. Landing pads can be integrated into one or more distinct chromosomal loci. For instance, distinct landing pads can be integrated into 1, 2, 3, 4, 5, 6, 7, or 8 distinct chromosomal loci, and one or more of the distinct chromosomal loci can be HI loci.

As referred to herein, the terms “site-specific integration site,” “recombination target site,” “RTS,” and “site-specific recombinase target site” are used interchangeably and refer to a short, e.g. less than about 60 base pairs, nucleic acid site or sequence that is recognized by a site-specific recombinase and that can be a crossover region during a site-specific recombination event. In some embodiments, a recombination target site can be less than about 60 base pairs, less than about 55 base pairs, less than about 50 base pairs, less than about 45 base pairs, less than about 40 base pairs, less than about 35 base pairs, or less than about 30 base pairs. In some embodiments, a recombination target site can be about 30 to about 60 base pairs, about 30 to about 55 base pairs, about 32 to about 52 base pairs, about 34 to about 44 base pairs, about 32 base pairs, about 34 base pairs, or about 52 base pairs. Examples of site-specific recombinase target sites include, but are not limited to, lox sites, rox sites, frt sites, att sites and dif sites. In some embodiments, recombination target sites are nucleic acids having substantially the same sequence as set forth in SEQ ID NOs.: 126-155.

In some embodiments, the RTS is a lox site selected from Table 2. As referred to herein, the term “lox site” refers to a nucleotide sequence at which a Cre recombinase can catalyze a site-specific recombination. A variety of non-identical lox sites are known to the art. The sequences of the various lox sites are similar in that they all contain identical 13-base pair inverted repeats flanking an 8-base pair asymmetric core region in which the recombination occurs. It is the asymmetric core region that is responsible for the directionality of the site and for the variation among the different lox sites. Illustrative (non-limiting) examples of these include the naturally occurring loxP (the sequence found in the P1 genome), IoxB, IoxL and IoxR (these are found in the E. coli chromosome) as well as several mutant or variant lox sites such as loxP 511, IoxΔ86, IoxΔ 117, IoxC 2, IoxP 2, IoxP 3 and loxP 23. In some embodiments, a lox recombination target site is a nucleic acid having at least 90%, 91%, 92%, 93%, 94%, 95%, 96%, 97%, 98%, 99%, or 100% sequence identity to the sequences found in Table 2.

TABLE 2

| Name | Identifier | Sequence |
|---|---|---|
| lox P | SEQ ID NO.: | ATAACTTCGTATAATGTATGCTATACGAAGTTAT |
| | 126 | |
| loxP 511 | SEQ ID NO.: | ATAACTTCGTATAATGTATACTATACGAAGTTAT |
| | 127 | |
| loxP 2272 | SEQ ID NO.: | ATAACTTCGTATAAAGTATCCTATACGAAGTTAT |
| | 128 | |
| loxP 5171 | SEQ ID NO.: | ATAACTTCGTATAATGTGTACTATACGAAGTTAT |
| | 129 | |
| loxP 2272(V) | SEQ ID NO.: | ATAACTTCGTATAGGATACTTTATACGAAGTTAT |
| | 130 | |
| pLox2+ | SEQ ID NO.: | ATAACTTCGTATAATGTATGCTATACGAAGTTAT |
| | 131 | |
| loxC 2 | SEQ ID NO.: | ACAACTTCGTATAATGTATGCTATACGAAGTTAT |
| | 153 | |
| loxP 3 | SEQ ID NO.: | TACCGTTCGTATAGTATAGTATATACGAAGTTAT |
| | 154 | |
| loxP 23 | SEQ ID NO.: | TACCGTTCGTATAGTATAGTATATACGAACGGTA |
| | 155 | |

As used herein, the terms “sequence identity” or “% identity” in the context of nucleic acid sequences or amino acid sequences refer to the percentage of residues in the compared sequences that are the same when the sequences are aligned over a specified comparison window. A comparison window can be a segment of at least 10 to over 1000 residues in which the sequences can be aligned and compared. Methods of alignment for determination of sequence identity are well-known in the art can be performed using publicly available databases such as BLAST (blast.ncbi.nlm.nih.qov/Blast.cgi).

In some embodiments, the RTS is a lox site selected from IoxΔ86, IoxΔ117, IoxC2, IoxP 2, IoxP 3 and loxP 23.

In some embodiments, the RTS is a Frt site selected from Table 3. As referred to herein, the term “Frt site” refers to a nucleotide sequence at which the product of the FLP gene of the yeast 2 μm plasmid, FLP recombinase, can catalyze a site-specific recombination. A variety of non-identical Frt sites are known to the art. The sequences of the various Frt sites are similar in that they all contain identical 13-base pair inverted repeats flanking an 8-base pair asymmetric core region in which the recombination occurs. It is the asymmetric core region that is responsible for the directionality of the site and for the variation among the different Frt sites. Illustrative (non-limiting) examples of these include the naturally occurring Frt (F), and several mutant or variant Frt sites such as Frt F1 and Frt F2. In some embodiments, the Frt recombination target site is a nucleic acid having at least 90%, 91%, 92%, 93%, 94%, 95%, 96%, 97%, 98%, 99%, or 100% sequence identity to the sequences found in Table 3.

TABLE 3

| Name | Identifier | Sequence |
|---|---|---|
| F | SEQ ID NO.: | GAAGTTACTATTCCGAAGTTCCTATTCTCTAGAAAGTATAG |
| | 132 | GAACTTC |
| F1 | SEQ ID NO.: | GAAGTTACTATTCCGAAGTTCCTATTCTCTAGATAGTATAG |
| | 133 | GAACTTC |
| F2 | SEQ ID NO.: | GAAGTTACTATTCCGAAGTTCCTATTCTCTACTTAGTATAG |
| | 134 | GAACTTC |
| F3 | SEQ ID NO.: | GAAGTTACTATTCCGAAGTTCCTATTCTTCAAATAGTATAG |
| | 135 | GAACTTC |
| F4 | SEQ ID NO.: | GAAGTTACTATTCCGAAGTTCCTATTCTCTAGAAGGTATAG |
| | 136 | GAACTTC |
| F5 | SEQ ID NO.: | GAAGTTCCTATTCCGAAGTTCCTATTCTTCAAAAGGTATAG |
| | 137 | GAACTTC |
| F6 | SEQ ID NO.: | GAAGTTCCTATTCCGAAGTTCCTATTCTTCAAAAAGTATAG |
| | 138 | GAACTTC |
| F7 | SEQ ID NO.: | GAAGTTCCTATTCCGAAGTTCCTATTCTTCAATAAGTATAG |
| | 139 | GAACTTC |
| F14 | SEQ ID NO.: | GAAGTTCCTATTCCGAAGTTCCTATTCTATCAGAAGTATAG |
| | 140 | GAACTTC |
| F15 | SEQ ID NO.: | GAAGTTCCTATTCCGAAGTTCCTATTCTTATAGGAGTATAG |
| | 141 | GAACTTC |
| Ff61 | SEQ ID NO.: | GAAGTTACTATTCCGAAGTTCCTATACTTTCTGGAGAATAG |
| | 142 | GAACTTC |
| F2151 | SEQ ID NO.: | GAAGTTACTATTCCGAAGTTCCTATACTCTCCAGAGAATA |
| | 143 | GGAACTTC |
| Fw2 | SEQ ID NO.: | GAAGTTACTATTCCGAAGTTCCTATACTATCTACAGAATAG |
| | 144 | GAACTTC |
| F2161 | SEQ ID NO.: | GAAGTTACTATTCCGAAGTTCCTATACTCTCTGGAGAATA |
| | 145 | GGAACTTC |
| F2262 | SEQ ID NO.: | GAAGTTACTATTCCGAAGTTCCTATACTATCTTGAGAATAG |
| | 146 | GAACTTC |

In some embodiments, the RTS is a rox site selected from Table 4. As referred to herein, the term “rox site” refers to a nucleotide sequence at which a Dre recombinase can catalyze a site-specific recombination. A variety of non-identical rox sites are known to the art. Illustrative (non-limiting) examples of these include roxR and roxF. In some embodiments, a rox recombination target site is a nucleic acid having at least 90%, 91%, 92%, 93%, 94%, 95%, 96%, 97%, 98%, 99%, or 100% sequence identity to the sequences found in Table 4.

TABLE 4

| Name | Identifier | Sequence |
|---|---|---|
| roxF | SEQ ID NO.: 147 | TAACTTTAAATAATGCCAATTATTTA |
| | | AAGTTA |
| roxR | SEQ ID NO.: 148 | TAACTTTAAATAATTGGCATTATTTA |
| | | AAGTTA |

In some embodiments, the RTS is an att site selected from Table 5. As referred to herein, the term “att site” refers to a nucleotide sequence at which a λ integrase or φC31 integrase, can catalyze a site-specific recombination. A variety of non-identical aat sites are known to the art. Illustrative (non-limiting) examples of these include attP, attB, proB, trpC, galT, thrA, and rrnB. In some embodiments, an att recombination target site is a nucleic acid having at least 90%, 91%, 92%, 93%, 94%, 95%, 96%, 97%, 98%, 99%, or 100% sequence identity to the sequences found in Table 5.

TABLE 5

| Name | Identifier | Sequence |
|---|---|---|
| attB | SEQ ID NO.: | CATCAGGGCGGTCAGGCCGTAGATGTGGAAGAACGGCAGCACG |
| | 149 | GCGAGGACG |
| attP | SEQ ID NO.: | ATGTGGTCCTTTAGATCCACTGACGTGGGTCAGTGTCTCTAAAGG |
| | 150 | ACTCGCG |
| attL | SEQ ID NO.: | CATCAGGGCGGTCAGGCCGTAGATGTGGGTCAGTGTCTCTAAAG |
| | 151 | GACTCGCG |
| attR | SEQ ID NO.: | ATGTGGTCCTTTAGATCCACTGACGTGGAAGAACGGCAGCACGG |
| | 152 | CGAGGACG |

In some embodiments, a cell can include multiple (e.g., at least four) RTS, e.g., multiple distinct RTS, and any useful combinations of RTS can be used. As used herein, the terms “distinct recombination target sites” or “distinct RTS” refer to non-identical or hetero-specific recombination target sites. For example, several variant Frt sites exist, but recombination can usually occur only between two identical Frt sites. In some embodiments, distinct recombination target sites refer to non-identical recombination target sites from the same recombination system (e.g. LoxP and LoxR). In some embodiments, distinct recombination target sites refer to non-identical recombination target sites from different recombination systems (e.g. LoxP and Frt). In some embodiments, distinct recombination target sites refer to a combination of recombination target sites from the same recombination system and recombination target sites from different recombination systems (e.g. LoxP, LoxR, Frt, and Frt1). For instance, in some embodiments, a mammalian cell can include at least two distinct RTS wherein at least one RTS is chromosomally integrated into an HI locus and at least one RTS is chromosomally-integrated into a chromosomal locus selected from Fer1L4 (see e.g. U.S. patent application Ser. No. 14/409,283), ROSA26, HGPRT, DHFR, COSMC, LDHA, or MGAT1.

A cell incorporating an RTS at an HI locus can be further processed to produce a recombinant protein producer cell. In addition to the RTS, a recombinant protein producer can include a gene that encodes a site-specific recombinase. A recombinase enzyme, also referred to as a recombinase, is an enzyme that catalyzes recombination in site-specific recombination. In one embodiment, a recombinase as may be utilized for site-specific recombination can be derived from a non-mammalian system. For instance a recombinase can be derived from bacteria, bacteriophage, or yeast.

In some embodiments, a nucleic acid sequence encoding a recombinase can be integrated into the host cell. For instance, a nucleic acid sequence encoding a recombinase can be delivered to the host cell by methods known to molecular biology. In some embodiments, a recombinase polypeptide sequence can be delivered to the cell directly.

Examples of recombinase enzymes as may be utilized include, without limitation, a Cre recombinase, a FLP recombinase, a Dre recombinase, a KD recombinase, a B2B3 recombinase, a Hin recombinase, a Tre recombinase, a λ integrase, a HK022 integrase, a HP1 integrase, a γδ resolvase/invertase, a ParA resolvase/invertase, a Tn3 resolvase/invertase, a Gin resolvase/invertase, a φC31 integrase, a BxB1 integrase, a R4 integrase or another functional recombinase enzyme.

In one embodiment a FLP recombinase can be utilized. A FLP recombinase catalyzes a site-specific recombination reaction that is involved in amplifying the copy number of the 2μ plasmid of Saccharomyces cerevisiae during DNA replication. A FLP recombinase can be derived from species of the genus Saccharomyces, and in one embodiment can be derived from a strain of Saccharomyces cerevisiae. In some embodiments, the FPL recombinase is derived from a strain of Saccharomyces cerevisiae. A FLP recombinase can be a thermostable, mutant FLP recombinase such as a FLP1 or FLPe. In some embodiments, the nucleic acid sequence encoding the FLP recombinase comprises human optimized codons.

Cre recombinase is a member of the Int family of recombinases (Argos et al. (1986) EMBO J. 5:433) and has been shown to perform efficient recombination of lox sites (locus of X-ing over) not only in bacteria but also in eukaryotic cells (Sauer (1987) Mol. Cell. Biol. 7:2087; Sauer and Henderson (1988) Proc. Natl Acad. Sci. 85:5166). A Cre recombinase can be derived in one embodiment from bacteriophage, e.g., from P1 bacteriophage.

In one embodiment, a mammalian cell can include an RTS chromosomally-integrated within an HI locus and the cell can be transfected with a vector comprising an exchangeable cassette encoding a gene of interest according to an SSI integration protocol. Upon integration of the exchangeable cassette within the HI locus a recombinant protein producer cell can be selected that includes the exchangeable cassette integrated into the chromosome. Selection can be, e.g., through the detection of the presence of a marker or can be through the detection of the absence of a marker using methods known to those skilled in the art.

An SSI protocol can be used to introduce one or more genes into a host cell chromosome. As used herein, “site-specific integration” can refer to integration of a nucleic acid sequence into a chromosome at a specific site and can also mean “site-specific recombination,” which refers to the rearrangement of two DNA partner molecules by specific enzymes performing recombination at their cognate pairs of sequences or target sites. Site-specific recombination, in contrast to homologous recombination, requires no DNA homology between partner DNA molecules, is RecA-independent, and does not involve DNA replication at any stage. In some embodiments, site-specific recombination uses a site-specific recombinase system to achieve site-specific integration of nucleic acids in host cells, e.g. mammalian cells. A recombinase system typically consists of three elements: two matching DNA sequences (recombination target sites) and a specific enzyme (recombinase). The recombinase catalyzes a recombination reaction between the matching recombination sites.

The term “matching” in reference to two RTS sequences refers to two sequences that have the ability to be bound by a recombinase and to affect a site-specific recombination between the two sequences. In some embodiments, an RTS of an exchangeable cassette matching an RTS of the cell refers to the RTS of the cassette having a sequence substantially identical to the RTS of the cell. In some embodiments, the exchangeable cassette contains a sequence substantially identical to one or two of the RTS chromosomally-integrated into the host cell genome.

As used herein, “transfection” refers to the introduction of an exogenous nucleic acid molecule, including a vector, into a cell. A “transfected” cell comprises an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to as “recombinant,” “transformed,” or “transgenic” organisms.

A vector (also referred to as an expression vector) can be any suitable replicon, such as a plasmid, phage, virus, or cosmid, to which another DNA segment may be attached to bring about the replication and/or expression of the attached DNA segment in a cell. Vectors can include episomal (e.g., plasmids) and non episomal vectors. For example, in one embodiment an episomal vector can be utilized that is removed/lost from a population of cells after a number of cellular generations, e.g., by asymmetric partitioning. A vector can be a viral or a non-viral vector and can introduce a nucleic acid molecule into a cell in vitro, in vivo, or ex vivo. Synthetic vectors are also encompassed herein. Vectors may be introduced into the desired host cells by well-known methods, including, but not limited to, transfection, transduction, cell fusion, and lipofection. Vectors can comprise various regulatory elements including promoters.

As used herein, the terms “exchangeable cassette,” “expression cassette,” and “cassette” are used interchangeably and refer to a mobile genetic element that contains a gene and can include an RTS. In some embodiments, an exchangeable cassette can include multiple RTS and/or multiple genes. For instance, an exchangeable cassette can include a GOI in conjunction with a reporter gene or a selection gene.

A GOI can include, without limitation, a reporter gene, a selection gene, a gene of therapeutic interest, an ancillary gene or a combination thereof.

As used herein, the term “reporter gene” refers to a gene whose expression confers a phenotype upon a cell that can be easily identified and measured. For example, a reporter gene can include a fluorescent protein gene or a selection gene. In one embodiment a selection gene can encode a product that confers to a cell the ability to survive in medium lacking what would otherwise be an essential nutrient. In some embodiments, a selection gene can confer to the cell resistance to an antibiotic or drug. A selection gene may be used to confer a particular phenotype upon a host cell. When a host cell expresses a selection gene in order to survive in selective medium, the gene is said to be a positive selection gene. Selection gene can also be used to select against host cells containing a particular gene; selection genes used in this manner are referred to as negative selection genes.

As used herein, the term “gene of therapeutic interest” refers to any functionally relevant nucleotide sequence. Thus, a gene of therapeutic interest can include any gene that encodes a protein the expression of which is desired the preparation of a therapeutic recombinant protein. Representative (non-limiting) examples of suitable genes of therapeutic interest include monoclonal antibodies, bi-specific monoclonal antibodies, and antibody drug conjugates (including blood clotting factors, well expressed mAbs where protein expression is limited at transcription, hormones such as EPO, immune-fusion proteins (Fc fusions), tri-specific mAbs, etc.).

As used herein, the terms “ancillary gene” or “helper gene” are used interchangeable and refer to a first gene that aids in the expression of a second gene or that aids in the stabilization, folding, or post translational modification of the product of the second gene or that creates a cellular environment that promotes the production of the product of the second gene. In some embodiments, the second gene encodes a DtE protein (or a portion thereof). An ancillary gene can encode, for example, an RNA (e.g., an mRNA, a tRNA, or a miRNA), a transcription factor, a chaperone, a chaperonin, a synthetase, an oxidase, a reductase, a glycotransferase, a protease, a kinase, a phosphatase, an acetyl transferase, a lipase, or an alkylase.

A GOI can encompass a gene encoding a well expressed therapeutic protein at a desired copy number. For example, a gene encoding a well expressed therapeutic protein can be at a copy number of 2 copies, of 3 copies, of 4 copies, of 5 copies, of 6 copies, of 7 copies, of 8 copies, of 9 copies, or of 10 copies.

As used herein, the term a “difficult to express protein” refers to a protein for which production is difficult. For instance, production of a DtE protein can be difficult because protein expression must be highly regulated, the protein is difficult to recover from the host cell, the protein is prone to mis-folding, the protein is prone to clipping, the protein is prone to degradation, the protein is prone to aggregation, the protein is poorly soluble, the protein is a membrane bound protein, the protein is difficult to purify, the protein is cytotoxic, the protein comprises multiple polypeptide chains, e.g. 2, 3 or 4 polypeptide chains, or any combination thereof. For instance a DtE protein can include multiple polypeptide chains that form a homo-oligomer or a hetero-oligomer to produce the DtE protein. In such an embodiment, the chains of a DtE protein can be encoded on one or more genes of interest that can be associated with the same or different RTS of a recombinant cell. A homo-oligomer or a hetero-oligomer can be formed through covalent interactions, non-covalent interactions, or a combination thereof. A DtE protein can also be a protein for which the expression of an ancillary gene is required to produce the DtE protein, or a protein for which a post-translational modification is required to produce the DtE protein.

A DtE protein can be a monoclonal antibody, such as a bi-specific monoclonal antibody or a tri-specific monoclonal antibody. Other examples of a DtE protein include an Fc-fusion protein, which is a fusion protein wherein the Fc domain of an immunoglobulin is operably linked to a second peptide. A DtE protein can be an enzyme, a a membrane receptor, and a bi-specific T-cell engager (BITE® Micromet AG, Munich, Germany).

In one embodiment, a GOI can be located between two RTS, i.e., with one of the RTS located 5′ of the gene and a different RTS located 3′ of the gene. In some embodiments, the RTS are located directly adjacent to the gene located between them. In some embodiments, the RTS are located at a defined distance from the gene located between them. In some embodiments, the RTS are directional sequences. In some embodiments, the RTS 5′ and 3′ of the gene located between them are directly oriented (i.e. they are oriented in the same direction). In some embodiments, the RTS 5′ and 3′ of the gene located between them are inversely oriented (i.e. they are oriented in opposite directions).

In some embodiments, a cell can include one or more additional GOI, and the one or more additional GOI can be chromosomally-integrated. A second gene of interest can be, for example, a reporter gene, a selection gene, a gene of therapeutic interest (e.g., a gene encoding a DtE protein), an ancillary gene, or a combination thereof. Additional GOI can be located within the same HI as the first GOI, within a second HI locus, or within a separate locus.

A second GIO can be integrated in a cell through use of the same or a different vector as is used to transfect a cell with the first GOI. For instance, a cell can be transfected with a first vector comprising a first exchangeable cassette encoding a first gene of interest and a second vector comprising a second exchangeable cassette encoding a second gene of interest. The first cassettes can be integrated into an HI locus and the second cassette can be integrated into the same HI locus, into a second HI locus, or into a separate locus. For instance, the second cassette can be integrated into the Fer1L4 locus. A recombinant protein producer cell can then be selected that includes both the first exchangeable cassette and the second exchangeable cassette integrated into the chromosome at the desired locations.

Beneficially, the SSI using landing pads located in HI loci in preparing rP expression cells can ensure that the pool of rP expression cells is homogenous in its genetic makeup. In addition SSI using landing pads located in HI loci to prepare rP expression cells can ensure that the pool of rP expression cells is homogenous in its efficiency. For example, the pool of producer cells can be homogenous in the ratio of a first helper gene to a second helper gene and/or that the pool of producer cells is homogenous in the ratio of helper genes to genes of therapeutic interest. Accordingly, SSI using landing pads located in HI to prepare rP expression cells can ensure a more consistent rP product quality.

The cell lines described herein, including prokaryotic and/or eukaryotic cell lines, can be cultured using any suitable device, facility and methods. Further, in embodiments, the devices, facilities and methods are suitable for culturing suspension cells or anchorage-dependent (adherent) cells and are suitable for production operations configured for production of pharmaceutical and biopharmaceutical products-such as polypeptide products, nucleic acid products (for example DNA or RNA), or mammalian or microbial cells and/or viruses such as those used in cellular and/or viral and microbiota therapies.

The cells can express or produce a product, such as a recombinant therapeutic or diagnostic product. Examples of products produced by cells can include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

Disclosed methods can allow for the production of eukaryotic cells, e.g., mammalian cells or lower eukaryotic cells such as for example yeast cells or filamentous fungi cells, as well as prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesized by the eukaryotic cells in a large-scale manner. In some embodiments, also disclosed are the use of microbial organisms and spores thereof utilized in microbiota therapeutics. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and methods can include any suitable reactor or bioreactor including but not limited to stirred tank, airlift, fiber, microfiber, hollow fiber, ceramic matrix, fluidized bed, fixed bed, and/or spouted bed bioreactors. As used herein, “reactor” or “bioreactor” can include a fermenter or fermentation unit, or any other reaction vessel and the term “reactor” is used interchangeably with “fermenter.” The term fermenter or fermentation refers to both microbial and mammalian cultures. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1 to about 100 or more bioreactors in each unit, for instance about 10 to about 90, or about 20 to about 80 bioreactors in each unit and/or a facility may contain multiple units having a single or multiple reactors within the facility. A bioreactor can be suitable for batch, semi fed-batch, fed-batch, perfusion, and/or a continuous fermentation processes. Any suitable reactor diameter can be used. For instance, a bioreactor can have a volume of from about 100 mL to about 50,000 L. Non-limiting examples include a volume of from about 250 mL to about 10 L, from about 10 L to about 500 L, from about 20 L to about 200 L, from about 500 L to about 5,000 L, or from about 5,000 L to about 50,000 L in some embodiments. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments and unless stated otherwise herein, the devices, facilities, and methods described herein can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

The recombinant cells can be mammalian cells as discussed previously and, in one particular embodiment can be CHO cells (e.g., a CHO-K1 cell, a CHO-DXB11 cell, a CHO-DG44 cell, a CHOK1SV™ cell including all variants, a CHO glutamine synthetase knockout cell including all variants, etc.), but the disclosure is not limited to these cells. Other examples of cells as may incorporate RTS in HI loci can include HEK293 cells including adherent and suspension-adapted variants, HeLa, HT1080, H9, HepG2, MCF7, MDBK Jurkat, NIH3T3, PC12, BHK (baby hamster kidney cell), VERO, YB2/0, Y0, C127, L, COS (e.g., COS1 and COS7), QC1-3, HEK-293, VERO, PER.C6, EBI, EB2, EB3, oncolytic or hybridoma-cell lines. Eukaryotic cells can also be avian cells, cell lines or cell strains, such as for example, EBx® cells, EB14, EB24, EB26, EB66, or EBvl3.

In some embodiments, the eukaryotic stem cells can be utilized. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). A differentiated form of any of the cells described herein is encompassed herein.

A eukaryotic cell can be a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii), the Geotrichum genus (e.g. Geotrichum fermentans), Hansenula polymorpha, Yarrowia lipolytica, or Schizosaccharomyces pombe.

A eukaryotic cell can be a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

A eukaryotic cell can be an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

A cell can be a bacterial or prokaryotic cell. For instance, a Gram-positive cell can be utilized such as Bacillus, Streptomyces Streptococcus, Staphylococcus or Lactobacillus. Bacillus that can be used can include, e.g. the B. subtilis, B. amyloliquefaciens, B. licheniformis, B. natto, or B. megaterium. In embodiments, the cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus Ohio 43210-1214.

A Gram-negative cell can be utilized, such as Salmonella spp. or Escherichia coli, such as e.g., TG1, TG2, W3110, DH1, DHB4, DH5a, HMS 174, HMS174 (DE3), NM533, C600, HB101, JM109, MC4100, XL1-Blue and Origami, as well as those derived from E. coli B-strains, such as for example BL-21 or BL21 (DE3), all of which are commercially available. Suitable host cells are commercially available, for example, from culture collections such as the DSMZ (Deutsche Sammlung von Mikroorganismen and Zellkulturen GmbH, Braunschweig, Germany) or the American Type Culture Collection (ATCC). In some embodiments, the cells include other microbiota utilized as therapeutic agents. These include microbiota present in the human microbiome belonging to the phyla Firmicutes, Bacteroidetes, Proteobacteria, Verrumicrobia, actinobacteria, fusobacteria and cyanobacteria. Microbiota can include both aerobic, strict anaerobic or facultative anaerobic and include cells or spores. Therapeutic Microbiota can also include genetically manipulated organisms and vectors utilized in their modification. Other microbiome-related therapeutic organisms can include: archaea, fungi and virus. See e.g., The Human Microbiome Project Consortium. Nature 486, 207-214 (14 Jun. 2012); Weinstock, Nature, 489(7415): 250-256 (2012); Lloyd-Price, Genome Medicine 8:51 (2016).

The rP producing cells can be cultured to produce peptides, amino acids, fatty acids or other useful biochemical intermediates or metabolites. For example, molecules having a molecular weight of about 4000 Daltons to greater than about 140,000 Daltons can be produced. The molecules produced by the cells can have a range of complexity and can include post-translational modifications including glycosylation.

Proteins as may be produced can include, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-la), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex′ recombinant human luteinizing hormone, dornase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21 S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favid, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 1311-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111 In-hEGF, AE-37, trasnizumab-DM1, Antagonist G, IL-12 (recombinant), PM-02734, IMP-321, rhlGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

Other examples of peptides as may be produced include, without limitation to, adalimumab (HUMIRA), infliximab (REMICADE™), rituximab (RITUXAN™/MABTHERA™) etanercept (ENBREL™), bevacizumab (AVASTIN™) trastuzumab (HERCEPTIN™), pegrilgrastim (NEULASTA™), or any other suitable polypeptide including biosimilars and biobetters.

Other suitable polypeptides are those listed below in Table 6 and in US2016/0097074. One of skill in the art can appreciate that the disclosure of the present invention additional would encompass combinations of products and/or conjugates as described herein [(i.e., multi-proteins, modified proteins (conjugated to PEG, toxins, other active ingredients).

TABLE 6

| Protein Product | Reference Listed Drug |
|---|---|
| interferon gamma-1b | Actimmune ® |
| alteplase; tissue plasminogen activator | Activase ®/Cathflo ® |
| recombinant antihemophilic factor | Advate |
| human albumin | Albutein ® |
| Laronidase | Aldurazyme ® |
| interferon alfa-N3, human leukocyte | Alferon N ® |
| derived | |
| human antihemophilic factor | Alphanate ® |
| virus-filtered human coagulation factor IX | AlphaNine ® SD |
| Alefacept; recombinant, dimeric fusion | Amevive ® |
| protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| Digoxin immune fab [ovine] | DigiFab ™ |
| Rasburicase | Elitek ® |
| Etanercept | ENBREL ® |
| Epoietin alfa | Epogen ® |
| Cetuximab | Erbitux ™ |
| Algasidase beta | Fabrazyme ® |
| Urofollitropin | Ferinex ™ |
| Follitropin beta | Follistim ™ |
| Teriparatide | FORTEO ® |
| Human somatropin | GenoTropin ® |
| Glucagon | GlucaGen ® |
| Follitropin alfa | Gonal-F ® |
| Antihemophillic factor | Helixate ® |
| Antihemophilic factor; Factor XIII | HEMOFIL |
| Adefovir dipivoxil | Hepsera ™ |
| trastuzumab | Herceptin ® |
| Insulin | Humalog ® |
| Antihemophilic factor/von willeBrand | Humate-P ® |
| factor complex-human | |
| Somatotropin | Humatrope ® |
| Adalimumab | HUMIRA ™ |
| Human insulin | Humulin ® |
| Recombinant human hyaluronidase | Hylenex ™ |
| Interferon alfacon-1 | Infergen ® |
| Eptifibatide | Integrillin ™ |
| Alpha-interferon | Intron A ® |
| Palifermin | Kepivance |
| Anakinra | Kineret ™ |
| Antihemophilic factor | Kogenate ®FS |
| Insulin glargine | Lantus ® |
| Granulocyte macrophage colony- | Leukine ®/Leukine ®Liquid |
| simulating factor | |
| Lutropin alfa for injection | Luveris |
| OspA lipoprotein | LYMErix ™ |
| Ranibizumab | LUCENTIS ® |
| Gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly |
| | LeuTech ®) |
| Somatropin [rDNA] | Norditropin ®/Norditropin |
| | Nordiflex ® |
| Mitoxantrone | Novantrone ® |
| Insulin; zinc suspension | Novolin L ® |
| Insulin; isophane suspension | Novolin N ® |
| Insulin, regular | Novolin R ® |
| Insulin | Novolin ® |
| Coagulation factor VIIa | NovoSeven ® |
| Somatropin | Nutropin ® |
| Immunoglobulin intravenous | Octagam ® |
| PEG-L-asparaginase | Oncaspar ® |
| Abatacept, fully human soluable fusion | Orencia ™ |
| protein | |
| Muromomab-CD3 | Orthoclone OKT3 ® |
| High-molecular weight hyaluronan | Orthovisc ® |
| Human chorionic gonadotropin | Ovidrel ® |
| Live attenuated Bacillus Calmette-Guerin | Pacis ® |
| Peginterferon alfa-2a | Pegasys ® |
| Pegylated version of interferon alfa-2b | PEG-Intron ™ |
| Abarelix (Injection suspension); | Plenaxis ® |
| gonadotropin-releasing hormone | |
| antagonist | |
| Epoietin alfa | Procrit ® |
| Aldesleukin | Proleukin, IL-2 ® |
| Somatrem | Protropin ® |
| Dornase alfa | Pulmozyme ® |
| Efalizumab; selective, reversible T-cell | RAPTIVA ™ |
| blocker | |
| Combination of ribavirin and alpha | Rebetron ™ |
| interferon | |
| Interferon beta 1a | Rebif ® |
| Antihemophilic factor | Recombinate ® rAHF |
| Antihemophilic factor | ReFacto ® |
| Lepirudin | Refludan ® |
| Infliximab | Remicade ® |
| Abciximab | ReoPro ™ |
| Reteplase | Retavase ™ |
| Rituxima | Rituxan ™ |
| Interferon alfa-2^{a} | Roferon-A ® |
| Somatropin | Saizen ® |
| Synthetic porcine secretin | SecreFlo ™ |
| Basiliximab | Simulect ® |
| Eculizumab | SOLIRIS ® |
| Pegvisomant | SOMAVERT ® |
| Palivizumab; recombinantly produced, | Synagis ™ |
| humanized mAb | |
| Thyrotropin alfa | Thyrogen ® |
| Tenecteplase | TNKase ™ |
| Natalizumab | TYSABRI ® |
| Human immune globulin intravenous 5% | Venoglobulin-S ® |
| and 10% solutions | |
| Interferon alfa-n1, lymphoblastoid | Wellferon ® |
| Drotrecogin alfa | Xigris ™ |
| Omaluzumab; recombinant DNA-derived | Xolair ® |
| humanized monoclonal antibody targeting | |
| immunoglobulin-E | |
| Daclizumab | Zenapax ® |
| Ibritumomab tiuxetan | Zevalin ™ |
| Somatotropin | Zorbtive ™ (Serostim ®) |

In embodiments, the polypeptide can be a hormone, blood clotting/coagulation factor, cytokine/growth factor, antibody molecule, fusion protein, protein vaccine, or peptide as shown in Table 7.

TABLE 7

| Therapeutic Product type | Product | Trade Name |
|---|---|---|
| Hormone | Erythropoietin, Epoein-α | Epogen, Procrit |
| | Darbepoetin-α | Aranesp |
| | Growth hormone (GH), | Genotropin, Humatrope, |
| | somatotropin | Norditropin, NovlVitropin, |
| | Human follicle-stimulating | Nutropin, Omnitrope, |
| | hormone (FSH) | Protropin, Siazen, Serostim, |
| | Human chorionic gonadotropin | Valtropin |
| | Lutropin-α | Gonal-F, Follistim |
| | Glucagon | Ovidrel, Luveris |
| | Growth hormone releasing | GlcaGen |
| | hormone (GHRH) | Geref |
| | Secretin | ChiRhoStim (human peptide), |
| | Thyroid stimulating hormone | SecreFlo (porcine peptide) |
| | (TSH), thyrotropin | Thyrogen |
| Blood | Factor VIIa | NovoSeven |
| Clotting/Coagulation | Factor VIII | Bioclate, Helixate, Kogenate, |
| Factors | Factor IX | Recombinate, ReFacto |
| | Antithrombin III (AT-III) | Benefix |
| | Protein C concentrate | Thrombate III |
| | | Ceprotin |
| Cytokine/Growth | Type I alpha-interferon | Infergen |
| factor | Interferon-αn3 (IFN-αn3) | Alferon N |
| | Interferon-α1a (rIFN-α) | Avonex, Rebif |
| | Interferon-α1b (rIFN-α) | Betaseron |
| | Interferon-α1b (IFN-α) | Actimmune |
| | Aldesleukin (interleukin 2(IL2), | Proleukin |
| | epidermal theymocyte activating | Kepivance |
| | factor; ETAF | Regranex |
| | Palifermin (keratinocyte growth | Anril, Kineret |
| | factor; KGF) | |
| | Becaplemin (platelet-derived | |
| | growth factor; PDGF) | |
| | Anakinra (recombinant IL1 | |
| | antagonist) | |
| Antibody molecules | Bevacizumab (VEGFA mAb) | Avastin, Erbitux |
| | Cetuximab (EGFR mAb) | Vectibix |
| | Panitumumab (EGFR mAb) | Campath |
| | Alemtuzumab (CD52 mAb) | Rituxan |
| | rituximab (CD20 chimeric Ab) | Herceptin, Orencia |
| | Trastuzumab (HER2/Neu mAb) | Humira, Enbrel |
| | Abatacept (CTLA Ab/Fc fusion) | Remicade |
| | Adalimumab (TNFαmAb) | Amevive |
| | Etanercept (TNF receptor/Fc | Raptiva, Tysabri |
| | fusion) | Soliris, Orthoclone, OKT3 |
| | Infliximab (TNFα chimeric mAb) | |
| | Alefacept (CD2 fusion protein) | |
| | Efalizumab (CD11a mAb) | |
| | Natalizumab (integrin α4 | |
| | subunit mAb) | |
| | Eculizumab (C5mAb) | |
| | Muromonab-CD3 | |
| Other: | Insulin | Humulin, Novolin |
| Fusion | Hepatitis B surface antigen | Engerix, Recombivax HB |
| proteins/Protein | (HBsAg) | Gardasil |
| vaccines/Peptides | HPV vaccine | LYMErix |
| | OspA | Rhophylac |
| | Anti-Rhesus(Rh) | Fuzeon |
| | immunoglobulin G | QMONOS |
| | Enfuvirtide | |
| | Spider silk, e.g., fibrion | |

In embodiments, the protein is multispecific protein, e.g., a bispecific antibody as shown in Table 8.

TABLE 8

| Name (other | | | Proposed | Diseases |
|---|---|---|---|---|
| names, sponsoring | BsAb | | mechanisms of | (or healthy |
| organizations) | format | Targets | action | volunteers) |
| Catumaxomab | BsIgG: | CD3, | Retargeting of T | Malignant ascites |
| (Removab ®, | Triomab | EpCAM | cells to tumor, Fc | in EpCAM positive |
| Fresenius Biotech, | | | mediated effector | tumors |
| Trion Pharma, | | | functions | |
| Neopharm) | | | | |
| Ertumaxomab | BsIgG: | CD3, HER2 | Retargeting of T | Advanced solid |
| (Neovii Biotech, | Triomab | | cells to tumor | tumors |
| Fresenius Biotech) | | | | |
| Blinatumomab | BiTE | CD3, CD19 | Retargeting of T | Precursor B-cell |
| (Blincyto ®, AMG 103, | | | cells to tumor | ALL |
| MT 103, MEDI 538, | | | | ALL |
| Amgen) | | | | DLBCL |
| | | | | NHL |
| REGN1979 | BsAb | CD3, CD20 | | |
| (Regeneron) | | | | |
| Solitomab (AMG | BiTE | CD3, | Retargeting of T | Solid tumors |
| 110, MT110, Amgen) | | EpCAM | cells to tumor | |
| MEDI 565 (AMG | BiTE | CD3, CEA | Retargeting of T | Gastrointestinal |
| 211, MedImmune, | | | cells to tumor | adenocancinoma |
| Amgen) | | | | |
| RO6958688 (Roche) | BsAb | CD3, CEA | | |
| BAY2010112 (AMG | BiTE | CD3, PSMA | Retargeting of T | Prostate cancer |
| 212, Bayer; Amgen) | | | cells to tumor | |
| MGD006 | DART | CD3, | Retargeting of T | AML |
| (Macrogenics) | | CD123 | cells to tumor | |
| MGD007 | DART | CD3, gpA33 | Retargeting of T | Colorectal cancer |
| (Macrogenics) | | | cells to tumor | |
| MGD011 | DART | CD19, CD3 | | |
| (Macrogenics) | | | | |
| SCORPION | BsAb | CD3, CD19 | Retargeting of T | |
| (Emergent | | | cells to tumor | |
| Biosolutions, | | | | |
| Trubion) | | | | |
| AFM11 (Affimed | TandAb | CD3, CD19 | Retargeting of T | NHL and ALL |
| Therapeutics) | | | cells to tumor | |
| AFM12 (Affimed | TandAb | CD19, | Retargeting of NK | |
| Therapeutics) | | CD16 | cells to tumor cells | |
| AFM13 (Affimed | TandAb | CD30, | Retargeting of NK | Hodgkin's |
| Therapeutics) | | CD16A | cells to tumor cells | Lymphoma |
| GD2 (Barbara Ann | T cells | CD3, GD2 | Retargeting of T | Neuroblastoma |
| Karmanos Cancer | preloaded | | cells to tumor | and osteosarcoma |
| Institute) | with BsAb | | | |
| pGD2 (Barbara Ann | T cells | CD3, Her2 | Retargeting of T | Metastatic breast |
| Karmanos Cancer | preloaded | | cells to tumor | cancer |
| Institute) | with BsAb | | | |
| EGFRBi-armed | T cells | CD3, EGFR | Autologous | Lung and other |
| autologous activated | preloaded | | activated T cells to | solid tumors |
| T cells (Roger | with BsAb | | EGFR-positive | |
| Williams Medical | | | tumor | |
| Center) | | | | |
| Anti-EGFR-armed | T cells | CD3, EGFR | Autologous | Colon and |
| activated T-cells | preloaded | | activated T cells to | pancreatic cancers |
| (Barbara Ann | with BsAb | | EGFR-positive | |
| Karmanos Cancer | | | tumor | |
| Institute) | | | | |
| rM28 (University | Tandem | CD28, | Retargeting of T | Metastatic |
| Hospital Tübingen) | scFv | MAPG | cells to tumor | melanoma |
| IMCgp100 | ImmTAC | CD3, | Retargeting of T | Metastatic |
| (Immunocore) | | peptide | cells to tumor | melanoma |
| | | MHC | | |
| DT2219ARL (NCI, | 2 scFv | CD19, | Targeting of protein | B cell leukemia |
| University of | linked to | CD22 | toxin to tumor | or lymphoma |
| Minnesota) | diphtheria | | | |
| | toxin | | | |
| XmAb5871 (Xencor) | BsAb | CD19, | | |
| | | CD32b | | |
| NI-1701 | BsAb | CD47, | | |
| (NovImmune) | | CD19 | | |
| MM-111 (Merrimack) | BsAb | ErbB2, | | |
| | | ErbB3 | | |
| MM-141 (Merrimack) | BsAb | IGF-1R, | | |
| | | ErbB3 | | |
| NA (Merus) | BsAb | HER2, | | |
| | | HER3 | | |
| NA (Merus) | BsAb | CD3, | | |
| | | CLEC12A | | |
| NA (Merus) | BsAb | EGFR, | | |
| | | HER3 | | |
| NA (Merus) | BsAb | PD1, | | |
| | | undisclosed | | |
| NA (Merus) | BsAb | CD3, | | |
| | | undisclosed | | |
| Duligotuzumab | DAF | EGFR, | Blockade of 2 | Head and neck |
| (MEHD7945A, | | HER3 | receptors, ADCC | cancer |
| Genentech, Roche) | | | | Colorectal cancer |
| LY3164530 (Eli Lily) | Not | EGFR, MET | Blockade of 2 | Advanced or |
| | disclosed | | receptors | metastatic cancer |
| MM-111 (Merrimack | HSA body | HER2, | Blockade of 2 | Gastric and |
| Pharmaceuticals) | | HER3 | receptors | esophageal |
| | | | | cancers |
| | | | | Breast cancer |
| MM-141, (Merrimack | IgG-scFv | IGF-1R, | Blockade of 2 | Advanced solid |
| Pharmaceuticals) | | HER3 | receptors | tumors |
| RG7221 | CrossMab | Ang2, VEGFA | Blockade of 2 | Solid tumors |
| (RO5520985, Roche) | | | proangiogenics | |
| RG7716 (Roche) | CrossMab | Ang2, VEGFA | Blockage of 2 | Wet AMD |
| | | | proangiogenics | |
| OMP-305B83 | BsAb | DLL4/VEGF | | |
| (OncoMed) | | | | |
| TF2 (Immunomedics) | Dock and | CEA, HSG | Pretargeting tumor | Colorectal, breast |
| | lock | | for PET or | and lung cancers |
| | | | radioimaging | |
| ABT-981 (AbbVie) | DVD-Ig | IL-1α, IL-1β | Blockade of 2 | Osteoarthritis |
| | | | proinflammatory | |
| | | | cytokines | |
| ABT-122 (AbbVie) | DVD-Ig | TNF, IL-17A | Blockade of 2 | Rheumatoid |
| | | | proinflammatory | arthritis |
| | | | cytokines | |
| COVA322 | IgG-fynomer | TNF, IL17A | Blockade of 2 | Plaque psoriasis |
| | | | proinflammatory | |
| | | | cytokines | |
| SAR156597 (Sanofi) | Tetravalent | IL-13, IL-4 | Blockade of 2 | Idiopathic |
| | bispecific | | proinflammatory | pulmonary fibrosis |
| | tandem IgG | | cytokines | |
| GSK2434735 | Dual- | IL-13, IL-4 | Blockade of 2 | (Healthy volunteers) |
| (GSK) | targeting | | proinflammatory | |
| | domain | | cytokines | |
| Ozoralizumab | Nanobody | TNF, HSA | Blockade of | Rheumatoid |
| (ATN103, Ablynx) | | | proinflammatory | arthritis |
| | | | cytokine, binds to | |
| | | | HSA to increase | |
| | | | half-life | |
| ALX-0761 (Merck | Nanobody | IL-17A/F, | Blockade of 2 | (Healthy volunteers) |
| Serono, Ablynx) | | HSA | proinflammatory | |
| | | | cytokines, binds to | |
| | | | HSA to increase | |
| | | | half-life | |
| ALX-0061 (AbbVie, | Nanobody | IL-6R, HSA | Blockade of | Rheumatoid |
| Ablynx; | | | proinflammatory | arthritis |
| | | | cytokine, binds to | |
| | | | HSA to increase | |
| | | | half-life | |
| ALX-0141 (Ablynx, | Nanobody | RANKL, | Blockade of bone | Postmenopausal |
| Eddingpharm) | | HSA | resorption, binds to | bone loss |
| | | | HSA to increase | |
| | | | half-life | |
| RG6013/ACE910 | ART-Ig | Factor IXa, | Plasma coagulation | Hemophilia |
| (Chugai, Roche) | | factor X | | |

### Example 1

Described is an example of the process of generating multi-dimensional maps of a genome by orthogonal methods, and then using that map or maps to generate a list of candidate HI loci for targeted integration of transgenes with predicted high expression and stability. The filtering process or algorithm employed to obtain the list of candidate loci using the multi-dimensional maps is summarized in FIG. 1 and described below.

Firstly, a reference genome assembly was constructed onto which multi-level genetic and epigenetic data was subsequently appended.

Hi-C data derived from the CHO-K1SV 10E9 Chinese Hamster Ovary (CHO) cell line (Zhang et al., Biotechnol Prog. 2015: 31(6) 1645-56), was used to inform de-novo assembly of CHO-K1 SV (ancestral cell line of 10E9) sequencing scaffolds initially constructed from short-read Illumina sequences. As a result of proximity-based ligation, Hi-C data is characterized by an increased density of contacts between regions residing close to each other on the linear sequence, and/or regions within the same chromosome. Thus Hi-C can be used to ascertain connections between previously isolated sequence scaffolds within fragmented reference assemblies. Over 310 million unique, valid Hi-C read-pair alignments from three biological replicates were used to cluster, order and orientate CHO-K1 SV sequence scaffolds via the published LACHESIS algorithm (Burton, J. et al. Chromosome-scale scaffolding of de novo genome assemblies based on chromatin interactions. Nat. Biotechnol. 31, 1119-1125 (2013)). The LACHESIS assembly comprises 1146 input sequence scaffolds and includes 90.52% of the original CHO-K1 SV sequence. The final assembly clustered input sequence scaffolds into 13 high confidence groups, with a length profile ranging from 12 Mb to 455 Mb.

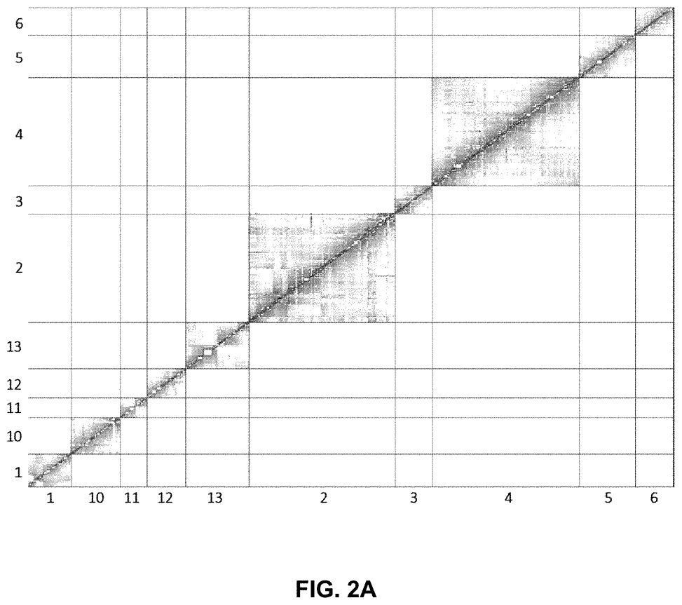

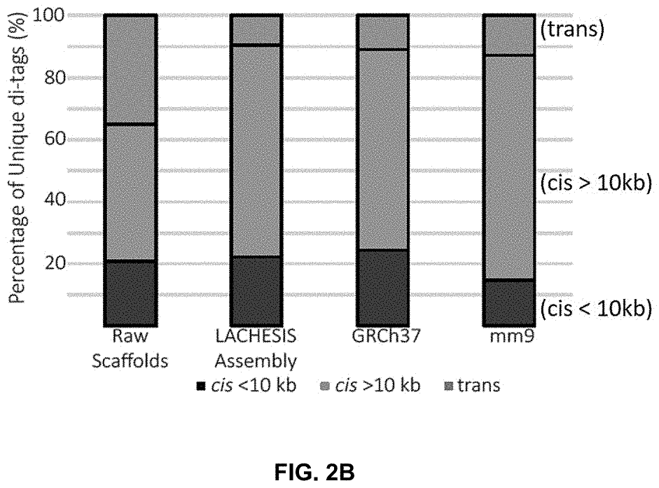

Hi-C data from the 10E9 cell line aligned to the LACHESIS assembly produced genome-wide contact maps (FIG. 2A) akin to those associated with the more established human and mouse reference assemblies and possessed a cis/trans ratio of valid read-pairs consistent with equivalent Hi-C datasets derived from human embryonic stem cells and mouse fetal liver cells (FIG. 2B).

Three replicates of paired-end Hi-C sequence data and Promoter Capture Hi-C (PCHi-C) sequence data, derived from the Chinese Hamster Ovary SSI 10E9 cell line (Zhang et al., Biotechnol Prog. 2015: 31(6) 1645-56), were individually processed through HiCUP version 0.5.9.dev under default parameters (Wingett S, et al., F1000Research 2015, 4:1310)). Mapping of uniquely aligned, valid read pairs to a sequence of interest was carried out using Bowtie version 1.1.0 (Langmead B, et al., Genome Biol. 2009; 10(3):R25) as part of the HiCUP pipeline.

Three replicates of paired-end ATAC-Seq sequence data generated according to a protocol described in Buenrostro et al. 2013 (Nat Methods 10, 1213-1218), and derived from the Chinese Hamster Ovary SSI 10E9 cell line were sequenced across two lanes. All resulting FASTQ files were trimmed to remove sequencing adaptor sequences in paired-end mode prior to mapping to the sequence of interest using Bowtie2 (Langmead B, Salzberg S. Fast gapped-read alignment with Bowtie 2. Nature Methods. 2012, 9:357-359) in paired-end mode and a maximum fragment length of 2000 base pairs. Subsequent BAM files corresponding to the same sample were then merged using a custom Perl script and alignments with a mapping quality score of less than 20 were removed from the sample merged BAM files using the Samtools view function (Li H., Handsaker B., Wysoker A., Fennell T., Ruan J., Homer N., Marth G., Abecasis G., Durbin R. and 1000 Genome Project Data Processing Subgroup (2009) The Sequence alignment/map (SAM) format and SAMtools. Bioinformatics, 25, 2078-9).

Published histone modification ChIP-Seq sequence datasets, derived from a suspension-adapted CHO-K1 cell line (Feichtinger J, et al. Biotechnol Bioeng. 113(10):2241-53 (2016)—Accession Code PRJEB9291), were downloaded and each FASTQ file was trimmed to remove sequencing adaptor sequences in single-end mode. Trimmed FASTQ files were then mapped to the sequence of interest using Bowtie2 in single-end mode and a maximum fragment length of 1000 base pairs. BAM files corresponding to different time points of the same histone modification were merged using a custom Perl script and once again, alignments with a mapping quality score of less than 20 were removed from the sample merged BAM files using the Samtools view function.

FASTQ files from three replicates of paired-end total RNA-Seq data, derived from the Chinese Hamster Ovary SSI 10E9 cell line (Zhang L, et al. 2015), were trimmed to remove sequencing adapter sequences in paired-end mode. Trimmed FASTQ files were then mapped to the sequence of interest using HiSat2 (Kim D, Langmead B and Salzberg S L. HISAT: a fast spliced aligner with low memory requirements. Nature Methods. 2012, 12:357-360) in paired-end mode under default parameters. Alignments with a mapping quality score of less than 40 were removed and replicate datasets merged within Seqmonk. RNA-Seq quantitation (RPKM values) was carried out using the RNA-Seq quantitation pipeline within SeqMonk (Babraham Bioinformatics—SeqMonk Mapped Sequence Analysis Tool by Simon Andrews), specifying that the libraries were non-strand specific, paired-end and that only reads overlapping annotated exons should be quantitated. The resulting quantitation was normalized for varying transcript lengths and log-transformed. Gene loci with negative log-RPKM values were all given a value of zero for downstream analysis.

### Hi-C Analysis

Filtered and mapped Hi-C BAM files from three replicates were merged using a custom Perl script. A Hi-C summary file was created from the merged BAM file using a custom Python script, before a HOMER (Heinz S., et al., Mol Cell 2010 May 28; 38(4):576-589. PMID: 20513432) tag Hi-C directory was created.

Topologically Associated Domains (TADs) were identified by subjecting the above Hi-C tag directory to the ‘findHiCDomains.pl’ HOMER script with a resolution of 5 Kb, a super-resolution of 25 Kb and a maximum interaction distance cut-off of 1 Mb. TAD boundaries utilized within the algorithm were the base pair extremities of domains defined in the output file.

Principal Component Analysis, mediating the identification of active genomic compartments, was carried out by subjecting the above Hi-C tag directory to the HOMER ‘runHiCpca.pl’ script with a resolution of 50 Kb and a super resolution of 100 Kb. The first two principal components were identified using a selection of 152 ‘actively expressed’ gene loci (determined by quantitation of steady state RNA-Seq data from the Chinese Hamster Ovary 10E9 cell line) as seed regions. Upon instances when the first principal component represented the segregation of different chromosomes arms, data from the second principal component was used. For all other ‘chromosomes’, data from the first principal component was used. ‘Active’ domains utilized within the algorithm were identified by subjecting an amalgamation of the principal component analysis data discussed above to the HOMER ‘findHiCCompartments.pl’ script.

Data input to the algorithm following this analysis included TAD boundary locations identified within the sequence of interest and coordinates of active compartments identified within the sequence of interest.

### ATAC-Seq Analysis

Peaks in accessible chromatin were identified in all three replicate ATAC-Seq filtered, merged BAM files mapped to the sequence of interest using the MACS2 ‘callpeak’ function with the following parameters; -q 0.01 --nolambda --nomodel --call-summits. The union of peaks that overlap in all three replicates, defined using the GenomicRanges Bioconductor package (Lawrence M, Huber W, Pages H, Aboyoun P, Carlson M, Gentleman R, Morgan M, Carey V (2013). “Software for Computing and Annotating Genomic Ranges.” PLoS Computational Biology, 9), were used subsequently within the algorithm.

### PCHi-C Analysis

Significant promoter interactions were identified from Promoter Capture Hi-C datasets using CHiCAGO version 1.1.3 (Cairns J, et al., Genome Biology. 2016. 17:127) under default parameters. A promoter capture RNA bait library was designed against the sequence of interest and a list of baited, promoter containing HindIII restriction fragments created. Prior to running CHiCAGO, aligned PCHi-C BAM files were filtered to remove read pairs not overlapping one of these baited, promoter containing HindIII restriction fragments using a custom Perl script. CHiCAGO was then run on individual replicate, filtered BAM files using default parameters. Cis interactions classed as statistically significant in at least two of the three replicates were extracted for further use.

### ChromHMM Analysis

Filtered, merged ATAC-Seq and published ChIP-Seq BAM files aligned to the sequence of interest were used to inform the production of a 17 state ChromHMM model (Ernst and Kellis M. Nat Protoc. 12:2478-2492 (2017). States 2 and 3 were attributed as being potential active enhancer regions, while states 11, 12, 14, 15 and 16 were assigned as regions having a potential repressive characteristic.

A list of potential active enhancer HindIII restriction fragments were defined as those restriction fragments first overlapping at least one ChromHMM state 2 or 3 region not within 2 Kb of an annotated TSS. These candidate restriction fragments were subsequently filtered to remove those also overlapping any of the ‘repressive’ ChromHMM state regions (11, 12, 14, 15 and 16) and/or a baited, promoter containing HindIII restriction fragment listed within the PCHi-C analysis section.

For the purposes of the algorithm, the list of cis PCHi-C interactions classed as statistically significant in at least two PCHi-C replicates were filtered against the list of potential active enhancer HindIII restriction fragments to give a set of reproducible promoter: predicted enhancer cis statistically significant interactions utilized within the algorithm.

The resulting potential HI loci discovered by this version of the algorithm are described in Table 1, with HI loci encompassed including these sites +/− about 5,000 base pairs to either side of the specific identified sites. The sites in Table 1 have been ranked according to predicted performance based upon a non-weighted additive summation of the ranking for each site with regard to proximity to the nearest TAD boundary, number of reproducible predicted enhancer cis interactions, and the steady state mRNA levels of the ‘associated’ genes.

Examples of where candidate HI loci sit within the 3D genome maps are provided in FIG. 3A for candidate HI loci SEQ ID NO: 3 and in FIG. 3B for candidate HI loci SEQ ID NO: 2 compared to that for the current industrially relevant FerIL4 landing pad in FIG. 3C. Of particular note is the spatial position relative to 1) TAD boundaries, 2) mapped peaks in open chromatin determined by ATAC-Seq, 3) the Promoter Capture Hi-C interactions mapped to the region, and 4) mapped epigenetic marks.

### Example 2

To demonstrate the ability of the method to identify HI loci using the procedure outlined in FIG. 1 and described in example 1, five of the top ranked candidate loci and five of the lower ranked loci were chosen for empirical evaluation. This was achieved by measuring the expression of a reporter gene cassette targeted for genome integration at the identified locus. Target loci were evaluated alongside two controls; a heterochromatic region and the 5′ flanking sequence of the Chinese Hamster Ovary SSI 10E9 cell line (Zhang et al., Biotechnol Prog. 2015: 31(6) 1645-56), Fer1I4 landing pad. The heterochromatic control region represented a peak in accessible chromatin not overlapping a HindIII restriction fragment involved in any reproducibly significant PCHi-C interaction. The peak also resides approximately 14 kb upstream of the ‘non-transcribed’ FbxI2 gene (Ref Seq ID NW_003613997.1, Genbank ID JH000418.1), within an inactive genomic compartment and overlaps a region populated by the constitutive heterochromatic histone mark, H3K9me3. The inclusion of these controls provided direct reference points for the assessment of candidate loci.

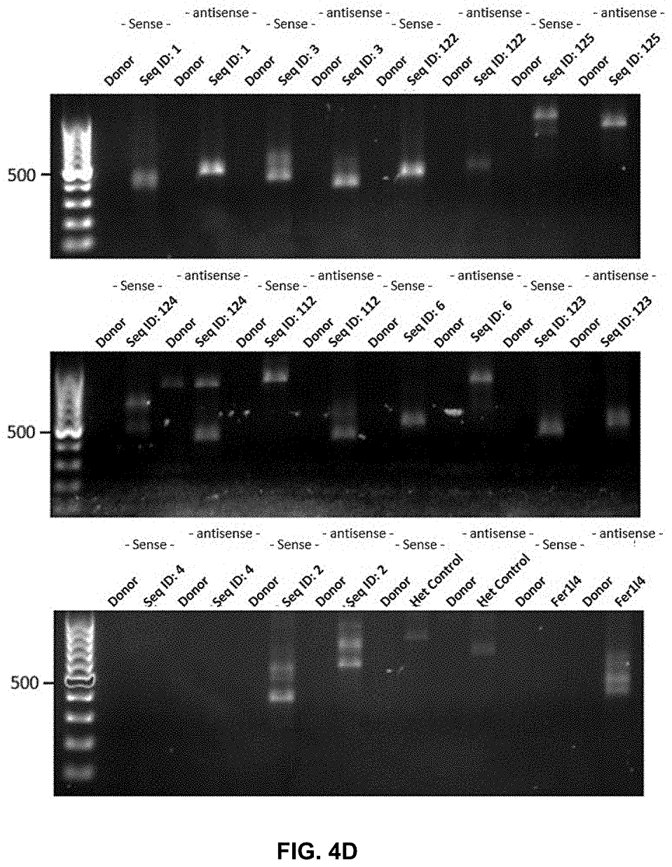

To test the candidate loci a custom designed GFP donor template plasmid was constructed, consisting of an eGFP expression cassette under the control of the constitutive CMV promoter, flanked by recognition sites for a custom designed ‘pseudo gRNA’ (FIG. 4A). The premise for using a custom designed pseudo gRNA sequence to mediate in vivo excision post transfection was taken from a published generic gene-tagging technique (Lackner et al., 2015; Nat Commun. 6:10237.). In addition to the reporter gene, the donor plasmid contained both the pseudo gRNA and locus-specific gRNA sequences (to target the CMV-eGFP cassette to the loci of interest), both under the control of U6 promoters and both including the gRNA scaffold sequence specified in Ran et al., 2013 (Ran et al., 2013; Nat Protoc. 8(11):2281-2308). Furthermore, the locus-specific gRNA cassette backbone consisted of two Bbsl restriction sites upstream of the gRNA scaffold sequence allowing incorporation of locus specific crRNA sequences using the cloning strategy outlined again in Ran et al., 2013 (Ran et al., 2013). The pseudo gRNA remained constant in all experiments, whereas the locus-specific gRNA varied to allow locus-specific targeting of the CMV-eGFP cassette.

After co-transfection of the donor and Cas9 plasmids, the Cas9 nuclease cleaves the CMV-eGFP cassette out of the donor plasmid as directed by the binding of the pseudo gRNA to the recognition sites flanking the CMV-eGFP cassette. The cassette should then be integrated at the target genomic loci by the cellular endogenous NHEJ (non-homologous end joining) machinery following target genomic DNA cleavage by Cas9 working in combination with the locus-specific gRNA.

For each candidate loci, crRNA target sequences were identified using an in-house CRISPR gRNA design tool that takes into account the propensity to mediate off-target genome cleavage. The top three ranked crRNA target sequences, each specific to distinct regions across the relevant candidate loci, were chosen. These sequences were then individually cloned into the donor plasmid downstream of the U6 promoter and upstream of the gRNA scaffold sequence at the Bbsl sites to create the final expressed gRNA for the target loci as outlined in Ran et al. 2013. For each target loci three separate donor plasmids were constructed containing the individual crRNA sequences. Sterile 5 μg donor plasmid libraries for each candidate loci were created by mixing equimolar ratios of the three constructed donor plasmids. These libraries were then transfected into Chinese Hamster Ovary SSI 10E9 cells along with 5 μg of a sterile Cas9-Puro plasmid (Dharmacon U-005100-120), giving a total of 10 μg plasmid DNA at transfection.

Chinese Hamster Ovary SSI 10E9 cells on days 2 or 3 of subculture were transfected with the donor and Cas9 plasmids by electroporation using a Bio-Rad Gene Pulser Xcell electroporation system, with a cell to DNA transfection ratio of 1×10⁷ viable cells in 0.7 mL CD-CHO media to 10 μg plasmid DNA in 100 μL TE buffer. The triplicate transfection cuvettes were then pooled into 30 mL pre-warmed CD-CHO media and left to recover. Cultures were left for a total of 13 days to recover prior to analysis. During this time, the culture media was changed on day 4 and cultures sub-cultured at a cell density of 1×10⁶ viable cells per mL on day 7 and day 10.

On the day of analysis duplicate injections of 20,000 cells from each cell pool were analyzed for GFP output per cell by flow-cytometry using the Guava easyCyte 12HT benchtop flow cytometer. In (FIG. 4B) the average percentage of GFP+ cells in each transfection pool targeting a specific genomic locus can be observed. The donor plasmid lacking any locus-specific gRNA was included as a negative control (‘plasmid control’), for GFP expression achieved from random, homology-independent genomic integration of the donor plasmid and/or expression from residual, transient plasmid remaining after pool outgrowth. In (FIG. 4C) the median GFP signal of the GFP+ cells for each pool is shown. From this sample of loci it can be observed that it was possible to identify HI loci that were approximately equivalent in expression performance to the Fer1L4 site, which has previously been identified by large-scale, random, empirical screening as a high-performing genomic site ((Zhang et al., Biotechnol Prog. 2015: 31(6) 1645-56)).

To demonstrate that on-target integration of the CMV-eGFP cassette had occurred in the pools analyzed above, genomic DNA from each cell pool was extracted using the GeneJET Genomic DNA purification kit under manufacturer's instructions. Targeted integration of the GFP expression cassette was assayed via PCR using a GFP specific primer and primers specific to the upstream and downstream sequences of each candidate integration loci. Aside from locus Seq ID: 4, targeted integrations at all candidate loci were confirmed (FIG. 4D). Using the primer combinations in this study, a sense amplicon from the FerI14 locus was not observed.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged either in whole or in part. Furthermore, those of ordinary skill in the art will appreciate that the foregoing description is by way of example only, and is not intended to limit the invention so further described in such appended claims.

1. A mammalian cell comprising a first recombination target site (RTS) chromosomally-integrated at a first high integrating (HI) locus, the first HI locus being within an active genomic compartment of accessible chromatin and within about 30,000 base pairs of a topologically associated domain (TAD) boundary, the first HI locus overlapping a region of the cell genome that interacts with at least one enhancer element.

2. The cell of claim 1, wherein the first HI locus comprises one of SEQ ID NOs: 1-125 or is within or overlapping about 5,000 base pairs of either the 5′ or 3′ end of any one of SEQ ID NOs: 1-125.

3. The cell of claim 1, wherein the first HI locus overlaps a transcription start site (TSS) within the active genomic compartment and wherein the TSS is optionally operably linked to an active gene, the expression or the lack of expression of the active gene being non-vital to the mammalian cell; or wherein the first HI locus does not overlap a gene locus and/or does not overlap and in situ endogenous promoter of a gene locus.

4-7. (canceled)

8. The cell of claim 1, comprising a second distinct RTS.

9. The cell of claim 8, wherein the first distinct RTS and the second distinct RTS are chromosomally-integrated within the first HI locus or wherein the second distinct RTS is chromosomally integrated at a separate locus, wherein the separate locus is optionally a Fer1I4 locus or is a second HI locus.

10-15. (canceled)

16. The cell of claim 1, wherein the mammalian cell is a mouse cell, a human cell, a Chinese hamster ovary (CHO) cell, a CHO-K1 cell, a CHO-DXB11 cell, a CHO-DG44 cell, a CHOK1 SV™ or variant thereof, a CHO glutamine synthetase knockout cell or variant thereof, a HEK cell, a HEK293 cell or an adherent or suspension-adapted variant thereof, a HeLa cell, or a HT1080 cell.

17. The cell of claim 1, further comprising a first gene of interest and optionally a second gene of interest and optionally a third gene of interest, wherein the first gene of interest and the second gene of interest if present and the third gene of interest if present are chromosomally-integrated.

18-27. (canceled)

28. The cell of claim 17, wherein the cell comprises the first gene of interest, the second gene of interest and the third gene of interest, and further wherein at least one of the first gene of interest, the second gene of interest, and the third gene of interest is within the first HI locus and at least one of the first gene of interest, the second gene of interest, and the third gene of interest is within a second HI locus.

29. The cell of claim 1, further comprising a site-specific recombinase gene, and optionally wherein the site-specific recombinase gene is chromosomally-integrated.

30. (canceled)

31. A method for producing a recombinant cell comprising:
mapping peaks in accessible chromatin of a cell genome;
identifying within the mapped peaks a first set of peaks within active genomic compartments of the accessible chromatin and also within about 30,000 base pairs of a topologically associated domain (TAD) boundary;
defining within the first set of peaks a first high integrating (HI) locus, the first HI locus overlapping a region of the genome that interacts with at least one enhancer element; and
inserting a first recombination target site (RTS) within the first HI locus.

32-33. (canceled)

34. The method of claim 31, further comprising identifying within the first set of peaks those peaks that overlap any transcription start site (TSS) for a gene, the expression product of which or lack thereof is non-vital, and defining a second set of peaks that overlap the genes and are downstream of the TSS, wherein the first HI locus is defined within the second set of peaks.

35. The method of claim 31, further comprising identifying within the first set of peaks a third set of peaks that that do not overlap any genes, wherein the first HI locus is defined within the third set of peaks.

36. The method of claim 31, further comprising transfecting the cell with a first vector comprising an exchangeable cassette encoding a first gene of interest and integrating the first exchangeable cassette within the first HI locus.

37-46. (canceled)

47. A method for producing a recombinant cell comprising:
mapping peaks in accessible chromatin of a cell genome;
identifying within the mapped peaks a first set of peaks within active genomic compartments of the accessible chromatin and also within about 30,000 base pairs of a topologically associated domain (TAD) boundary;
identifying within the accessible chromatin regions of the genome that interact with at least one enhancer element;
defining within the first set of peaks a plurality of high integrating (HI) loci, each HI locus of the plurality overlapping an identified region;
integrating a recombination target site (RTS) into a plurality of cells; and
selecting from the plurality of cells a cell comprising the RTS integrated at an HI locus.

48-49. (canceled)

50. The method of claim 47, further comprising identifying within the first set of peaks those peaks that overlap a transcription start site (TSS) for active genes, the expression of which or lack thereof having a non-vital function, and defining a second set of peaks that overlap the active genes and that are downstream of the TSS of the active genes, wherein the HI loci are defined within the second set of peaks.

51. The method of claim 47, further comprising identifying within the first set of peaks a third set of peaks that do not overlap any genes, wherein the HI loci are defined within the third set of peaks.

52. The method of claim 47, further comprising transfecting a plurality of the selected cell with a vector comprising an exchangeable cassette encoding a gene of interest and integrating the exchangeable cassette within the HI locus and selecting a recombinant protein producer cell comprising the exchangeable cassette integrated into the chromosome.

53-57. (canceled)

58. The method of claim 52, further comprising inserting one or more additional RTS within the cell, wherein the gene of interest is located between two of the RTS.

59-60. (canceled)

61. The method of claim 47, further comprising ranking the HI loci.

62. The method of claim 61, wherein the HI loci are ranked according to one or more of expression level of one or more genes associated with each locus, distance from each locus to the nearest TAD boundary, number of predicted enhancer interactions at each locus, and expression level of mRNA of one or more genes associated with each locus.


SEQUENCE LISTING (155 sequences)

SEQ ID NO: 1 - DNA, 572 bp, Cricetulus griseus
cgtgaagagaaacttctttaggtaactcggagaataccttattgtttggtttctttgaacagcatttcgggtttgttgattatgcttgtttatttcaccaccccaacttttatatgtgtatattcacatgacgttttttgtcacagggttcttcaattttccttaaaaatcttgggaataatatggcgctggtggagttgtttcaaaaggtcattccttcgagccggattcgaaccagcgacctaaggattaccatgactaacctacagtcctccgctctaccaactgagctatcgaaggaagacaatagtgctttctttaaggcttataaagtagctggtggatttgttgcactgcctttgattcattttaaaatggacgatatggaaatgtttttgaggtactagaatattttaaaagtaaattagctagtgaccacacaacaggtatttaagaacatgtttgagatgtgtgcttttcaatgtatacgtagttcacatagattccccctcccccccgccccaaaatgctcctagaaacgttcatgcactgaatccttacagaccagatca

SEQ ID NO: 2 - DNA, 459 bp, Cricetulus griseus
acagatgtccatgctaacagcattttcctggttgtatatagacaaccacattttttgagccttttatcacagtgggggaagcaattactcacagaaactttgcttaaacaaaactgaccaattagtcatacatatgcaaagtaaataaaggagtaaaccctgagagaatctgcctggggcacaagggtctatttctacccttaaggcccaggtctccttaagcactcttcagttactgtagtagccaaaggtcaagccaggcagcagcaccaggtaaatgttgttaagtgtgtggatttcttccccaagccctctcactgtctttgcacatttgggtggtaactctcctctgtcaaacccctgctactatgcctgttcaccccacactttcattcagaaaaacaggtgttcctgttaagatcagctgtctcaaactgccaaatagctcagatccctaca

SEQ ID NO: 3 - DNA, 618 bp, Cricetulus griseus
tcacctctagcaaatatgtatataaatatatgcgtatatatatttcatattctacttttcagttatcagtatctgagggccgcctttggttgctctaactcctggtgtcctcggcacaaatgaggtcctgactagtaaaataacacccatccattgaagaaagagggagggggaaggtcgccgccattctctcctcccagatggcaatctcgcagagcctcttcctcccgccgagcgggctgagctaagtggacagccccgcctctgcttccccgcgcccggacttgtcagcagctgcgcgcttttgcatctggcgccagttttgacctcggctacgtgattggtcggctccgcttcaatcaacgtcacaagtcccacccacccctcagtcctcgctccggggtcactccaggagctgggcacgtcagcgttaaggcgaccggaggtggggaaagggagtctggtgagattgagtgcttcctgcaaacccacacagcagtctgcatcagactgacaaaaacgcaggcacagcacccacagcccctgcacagccgtaccaggtcatgcacatttttaatccagatggaaagggaaaatagtatttttaggcctgtttta

SEQ ID NO: 4 - DNA, 555 bp, Cricetulus griseus
gtcgctgtgtaaaagttggcggggaagccgagtcggcgggcgagcacccgctgtagccgccccgccccgccccgccccgaggatggcgtttggagcagcctgggctccacagcgcgagacaggccagccggggttctagacatacgtggtcccggtgcaaaaaattaaaagataaccgaactgacgcacagactgacaaaataatctgaaactgactctagacgtgcgggcaaaatctcactgttttcaacctgaagatgactccttagctgggtgagaattgaggggaaggttttcccatcagactcagatattcacaagactcttcgaaattgaatgtactccaaccgtttttatgaagcccaggtttgtgtgtgtgtgagagagagaaagagacagagagtgttccttccctattcaaaaaacaagcatttaatgccctatgtacgtatcaattacacatagtacttttcattgttacattttcatatatggatatgtatgtctcctctaccctaaaacctattcctctttccagccagacctctgtgtgtg

SEQ ID NO: 5 - DNA, 476 bp, Cricetulus griseus
tgctctctctcacatagcccagctgtcccctttgcttgggagactttggctagtcttattcctccctggactcacattacctctagctacaacaaagcttcttggcagaagcccacaagcaggtcttcagtgcaaagcatcctgtggttccttctgctgaccctacagtccctggaggccttgcaaatactgcagacaaagccagctgcagctcagaactgcctgcctgttctatagaggcagcctatggaactgacagcctctgcagatctgaactgcagatcaactcaggagccctgaaacctgactccaaatacagcagcagatgcactgccaaaaggaagtgcggcggcggcgttgtttgctgaaggtttccattctcccagccttagcccctgagtttaactagtccctcaggaggccacagaacaaaagtgaccagtgcctcctatgacagcagtctatccttctaggac

SEQ ID NO: 6 - DNA, 1134 bp, Cricetulus griseus
tagtaaagaagtgaagaagtgtccaagcattgaactaagggacccaaactcgaacacataagcacacaacctcatagcaagggtagtaccgtgtcaccaccacgcgtggctcaaaggtcggggggtaagatctacctgcgggacgcaaatcaatgggtgaaaggttaacaggaagtggctacaagactagaggttcttaatgtgtcacagcatgacagaggaggcgttactaaaatagttttctcaacaccctctcagctggtaaagccataaagagtctgtaatatctggaagaaaacaaaaacaaaaacaaaaacaaagcatgattctcagggttgctgcccagacagcagccacaacgggagataacaaaggcgcatgcgcggcatccgggtttccggatgcgcgcctaaggccgacgtggcaaaggggtggctcccccagtcctacagcatggggcggaacttccggggacggcacgccagctccctcggaaccggaagtggagcctgggatccttgacgttaggaacgaagtcgaacctggatctggagccgggtgaggagtggcatcgcgaggttgcgcggtggggaggaatgctgaggagaggccagggtatttctggtctggccgtggtccggttttagaggaaagggaggagcctggaggcggggtctgggaagagaccctcacctcccgcatccgtgaccggcgatcggtcccagacccctcccttcacctcctggcccaggttccggctctcgcctcgccttcccggaatgttgaagggctttagaagcttggtctttactgcttaatgaaccggccctcatcgtggccctagaggctggcgtggcttgctgatctcgatccctggagctcccaagcctctgcatctcgggtactttcagagttctaggaggcttgccccgcccctttgggttgtcaccctcgactgtccacctagaatggagtcaagattggagtttgactccaagcttattgcgttgctctctctcttgacctgaggaagttaaagtgagagggacgtctcccaagctcactgtcagtggcatgtgtaaggtttagggtttggaaggacagttcctaaaatggctttgactttacagagtt

SEQ ID NO: 7 - DNA, 1120 bp, Cricetulus griseus
gtagagaggcctccttgctctctagctccctcccctccacagcctctgacctacacaagcagctgaggggtgggggtggggagatggagaggagagaggaaatgagaggggtgaggagaggaggcagactcatgcctggcctcactcttctggctttgctgaggagaggatggcagacaggagagctgttaaggtccaggacagtctcaggagcccatcaagtaagctcaagccaagctggatgataagccaggccagctggggatgtttgcttaagagatgcttgccagggcagtcgagccagaggaaataaaatgtgtctcacagaacctcggtaggcagggaaggtgtgtctcccccgcccccccccccaggcctaaggaagacctgggtggctgactttggtcttgtccagctgtggagcagaggtgagccaaagcctgaggtctgtatggctgagaggaagagctcagaagcccagcaaggatggtgaggtctctcagggggcaatcgccggcctcttggcactgataacaaggaaactggtttattgctggattttgttcggattctctccgaaaaccacataagcctcttcctccccacaggccctcagctggcatagtttcccatggccactcatcaccctgtgccactgtaacacctggactcttgtttctctctgattcccaaggcttctgtggcaaggagtgacatccacccagtacctggcctcagggctggggaacccagggcttagagccccaggcttcagcttcaggaagtttcccttgcctttgccttggaaagcattgtctaaaggcagctgagattgtgctatggtgctacatggaagtgtcgccctcttcaggcccagctgagagatgcctcgatagtcactagtacttccagaggacccaggtttgggttacaacgcccacatggtggtttgaacactcctgcaaacacatcagttgctgcctacactctgggctgctaaaacaaggtgtcatgtcataaataacagctgttactgaaccaaggaatctgtgatcaaggtgagggcctgccctccttgatgactttgatgtgtcctgacagggtgcaaggggccaagcct

SEQ ID NO: 8 - DNA, 426 bp, Cricetulus griseus
gttcggctccgggacctcggccgcccgagccgcctgacttgcccgcccaccggcctggctttccagtgcccgcagcctccctccgaagtgcatggacccggggcggtgaccgggagtggggtgggaggccaggctggggcgcaggccagagcggccgggcctccgggcgcgggagggaaagggcgccccctctcctctctggaacgtgtcaatgctttgcacttggggccggcatgaggctggggggccttccccaaggccctgggacccgggcgccccctgcctcgggcactttcggtgggtcagatcggccctccaggctttccagccctcgcccaggcagtggcccgcgcgggcggcggggtgggggccaggccagggggaggggtctcggggcccccctgcctctcattggttaatatttta

SEQ ID NO: 9 - DNA, 383 bp, Cricetulus griseus
gaggtgggagggacagcacgattcacaaacaacataatttgtggagcttgtcttagacaggatagcacctgaatggaaagagaggcttccctttagagcctggaaaagagcttcattaagaatatgaatgagggggctggagagatggctcagtggttaagaatactaactggtcctccagaggtcctggttcaattgccatctgtaatgagatctggtgccctcttctggcctgcagggacacaagcagacagaacattgtatacataataaataaatgttaaaaaaaaaaaaagaagaatgagagagcaatgagtagagaggcagacagcatggtgggtgggggaggcagctagaggcagacctcagaacatctctccagc

SEQ ID NO: 10 - DNA, 1562 bp, Cricetulus griseus
  modified_base (616)..(903): a, c, t, g, unknown or other
acagagaaaccctgtctggaaaccaaaaaaaaaaaaaaaaggaaaaagaaaaatcggtacagagactgagtcctaaccactaacgaacagcactgactatgaagtctttttcccccaagccaaatgagtcaactaacagtgaaatagacaaatgccttcagttccctgaaaacccttgggtccaagcccagtgagtgaacttcacctccaagtccctaaactcttgcctcgcatgaatattagcataatccctaccacccgatttcagatttcatcaggccaaactttaaaaaatctgtaagtgatctcagccctgcaggcccatccttctaccaggccaatagaggggaaaccctgaaatctgaacgcgctcccactatcactaccaagcgaggcagggaagtggcaagccgggtcgggtgctagccacatcccgcattggaatccctgtccttgtgtgagctctcaattcttactctggtgcgtccaaaacactctcttgcccctgacagttccagcgaccatatttactcccaccggggacccccagatacggcacaactatttgtgagccctggcctgaggcagaccctcgtggctcctcttcctcatccannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccacctggagccgccaaaggggcgggcacctgcgggggtgggcggagtcttacgtcactcgggtttctgtccagtaggatggcccgaggtcctgtcagtctgcggcaccgccctctcacgcactggcgggggcggggcccgctgcgcgtgtggccctggaagagggtaaggcctgatggaggaggcgccaccttgaggtcaagctgctaggtggggcctcagggtcacccctccccgggtgggtcgtcgcgacctgtgcctgagcggagccggacgggggcactgtgtcttcaggtgatggatttcggaacacccacgggtgtggttgggaaagacccgcgggtctctaggtagagatccctccggggcaggcctgtctcttaggttagcctgtttcatctctgatacctgccagccggtgcctcaggcttcctctgtcagctgtcttccagtctcaaggttgagaagcctgccgggttgccttttgcacagtcacaagtgaaagaagaaatgcaagcaaaaggggttggataacccgaggttgtgctaaatgtcacagtgagaatgggtctaggtgaggaaccaaaaattaagattaataaaagtttttaaacttaggccctgaaaagagaatgtccaaggaaaatttaactccttat

SEQ ID NO: 11 - DNA, 2319 bp, Cricetulus griseus
ctaaacagagaaaccctgtctcgaaaaaacaaagcaaaacaaaacaaaaacttagaggaatgctgcagggttgtggttattttccttcaaatccaaatctgtttctctccctttccccttttcttctgtgtcttgggctttcatatagtcctgagactctagacaatgtccccttggtcacagatgtcccttttacgagcaggggatgaaattcacacttgttattttgggtgcttcacctggaagtcaggttttggttcatgctatgaaagagccttgtcaaagaactacatgcagagacctggccaacacacctagattcctggtccataaaatagggcaatttttacttttagtgacgccttatttcaatcatgcaaaagagcttcagtttggagtgttagattctaatgaaaccttgatgccaagactgataaaaaaaattggctagaaaacttggcagaatttctcagtcagccatctgcctaggaatgaccttgttatcaaggccaataccaaaaaccaccctggatacaaggcagcagtgggacccctctgggaaaaggtctggtaagctcattgtagagccctcagacaggagacacttccactgcgaagggattgtctttgcttcttccttcccacaaaagcatacttctgatgcactttgtttcttgggaatccggggtgagaaactctaaagaatgtatagcaggcacagacctgtgtcagcctagagtgagatggggaagggtgggcgggcctagggagagggttgccaacacaaatgtcttatggggacttggaagttttgccaggaggctgacagggatgtgcccttgtctacagaaggtgcctggttccccctggtctaggctgaggaatctggagtcatgggaaagggcagagaaaactgctgcagggaaggactagaatgcaaaagaactaggcgaatgccaagggactactggaatttcccaggacctgggaggtgagggtgagaagccagagggccctcagaggaaaaggaaggggtgggggtgagttcaccgtacctctactcagttcctgattactgtgctgggtagaagggatcctctggtgtctggggcagaaggtgagtatggagccatggcttgagaatttcagcacaccaaattcactgtaccatctgcccaaaggttgtgggcatccgatcaattcacagacaatgcaagacacactggagttggaggtggcccttccacggtactccattctaactacactgatggccactgtcttccctgcctcagtaacacaggcattgaggttcactagtacttaggcttctcattttaactttcccaccttttctaagtccccaggtggattgggtgactttgtacttccagaaagagctgcatgcttgccttattgtgtaacatcccatcgtgttgtaatttctggctcacatctccatctccctgccatctgcattcctggaacaggaactgttcttgtctgtctgtcatggccaagccaaaggctctcactgagtagacagatgctcacatacccacaaggatcactccagcattgagtatcccttagggactagggtcaagagaacctagtactgtgtacctcctatccaatgggaatgcactttgccaccgcctatccacactgccatctcccaagaagctgccaagaatggcctttccaactggcaaagggatatgctttccttcctggaaggttttggctggagaactgccccttgcttctaagttgctggggccacacaaggatgaagttgtatcagccctagccttgttaccgtttcccaaagcttgtgtcatccacctcttatcttctccaagaacatctggcagggaccaggtttctccaatcctgggtagtagagatgaggggtgggattggctaatgcctctggtcacctggccacctttagctctacttaaccaacacaaatctacagtccaccacattgaaaaaagaacatgcctagacttggtatgatgggtgcttgctgtttttttttttttttcttctagagagagtttctctggctgtcctggtactcactctacagaccaggctggcttcaaactcagatctgcctgtctctgcctccagagtgctaggattaaaggaatatgccaacatgtctgactaggagtgcttgcttaacatgtggatggtcagttcccaacacacatgcacacacatctataaagccccaatctgaacaaaaccttaacccccttcttaggcaagaatggctacttcacagccttccccacagcccaggga

SEQ ID NO: 12 - DNA, 1501 bp, Cricetulus griseus
cgcactaccgatcctgctggacgacgtgcgctgcgcgggcagcgagcgcaacctgctggagtgcgcgcacgctggcgtgggcacgcataactgtaagcacgaagaggacgctggcgtcgagtgcagccaccaagacccggacctatagccaggcgtcacctgcctggctgtgccaccctgcgaagatggagccgagaatggccccttcggtctgcgggcaccctgtgcagcacgcacaaaccatggtgaagggcccaagagacggttcctcttcatgtgacctctgtgtctccctgtgtgtgtgtgtggtttgtggataagatctcttatctctcagcactccggagagggagagcgggggcagggactattcaggagtggggtgggggaggctggctgtctttacagtgttagactagggaaggagtcaggcgtcccctgccagggctgagtgacaggctccagaccaccaagagtgcagcctgagaaacagggccagagcgtaggcgggtgacccagcagttccaagcactgcagaaaaagaacaggcagtctgaaagccacagcggaggatcttatagggtcagggggcctgtggccatttgcagcccatcagggatgaggctgctgaagggaattaagagcccatgagggaaagcagactagagggcctggaggaccttggaggccacttgctgcgccctgcaggcaccctggaaaggctactgagtcccctccatccctgaagcctgaccattgcgtattcctgagacagaagcacccttaggctcacactcggtccctttggcctctgggccccagttggggggatgggggtggacctctactttcatcctctactgtctccatcacctcccaggggatcctcagactcccaggggatcctcagattcctccgccagatctgtgggctaagaagcctgtgggacctcaagactactcatctcaacaatttgactatttgcaatccttcctccttttatcacccaccacacacctggaagctgggatgccaggtgagggacagagtgaagagggcggggccttcagcttcactcctgccctgtgggaagagcgccttgctcacgggaggcctcagggtagctctggatagttgtaatttagcttgagataggggtggagccatgaaaacaaatgagtcaccaggcagggaggacaggacgtgacctgtgtcaggcagttgtggccagggatctgggttaggtttctggcccaacctttgcaggaaaatgtcattcctgcactgagttctgatctgaagaggcctgtgtgctgttgcaggcaaaggaactggtgcaggctcaggctgtgtgctgtgtgctcctatccatcactgctgcgaggggaacgggagggtcgccctgggctgctctgcagctttggaggctggttgttcccggaaataaataagaaagttactgaactgttaggaaatgacaggaaaggaaagcatcgcttc

SEQ ID NO: 13 - DNA, 662 bp, Cricetulus griseus
catcagtagagtgggagccagatgggtttcctcctcagatgacagtcagtagccctggaacttctgacttgtttctacaattgcagtacaacactaagaaaaacttttattctctgtaactagatattgtgggccaagaaaaccagcattttttttttttaaccttaccttggcagttggcaaatgaatgaccatggtgaccccgccctgcagagcacatgatcagccacagctgtgcatgttttttaggatgactgtgccaggacagagctccaagccactgggtggagcaatgtgcccctgggagaaggctcatgacatttcccctatgatttcagaaaagaaaaaaattcctcttttttgcttatgagttgtttctgcacaatgtcaacagaaaatttccttgtgtttatgcatgtccacgcatgaatgtggaaaatgtcagcctctgctacagcgggataaatacacaccacatttgacctcagagtgaaggtcaataagacacataccactagttagctgctttcttaacctccgctttttgcttttcccttcataacaagtctaattgtatttgggctggtaacaaatccaaccaaatgacaattcctcagctcctctcagcgaggtagactccagccaatgagaccagaagtgct

SEQ ID NO: 14 - DNA, 685 bp, Cricetulus griseus
cccctgaggtgctagggtggtgatttcagcctcccacagctcttagtggggctggtctaggggcaggcgtggtgtttggggtgctggtgggtatgcagtagcagcttgagagaacttggtcttctcttgagcccttcctcatagccttccagagtcagctggtcctactgcaccagaccagtagggctgctcactatgacagtccatgccaaatggctccctgtgagctttcctcggctctgaagccttctactgtatgagtcatacttatcctgtcgcagccctagaggctactggagactcattttatggatttggttcagcagcatgttttgaaggcatccaggatcttccatcccagctgaatagacagctgaccgcagtgtactcagtggttaagtgagtgagtgccctgtttttctgccagagtgggagcaaagggtgaggaggcagcagatgcttcaggtgatcgagcagatggaatagggagggaggagatccacagcatgaccttgcttggaaggttttctgaggtcaggcagaggggtgaccagtcaagaggtgcagatcttctcattagtggatgagtgtggaggttgattactggacgtgaggtgatatggagcctcatgtcagccacccactgcggtttcccagttagctggctgaggtcctggcctgaagc

SEQ ID NO: 15 - DNA, 909 bp, Cricetulus griseus
aaccaaattaaattgtgtgttgtgtaatagggcaacttcaaaaggaagtcacaaacttctttagatccccaaagatgacacctcaagaccagcacccagcaaagctcagtgctctagcccttcccaaccctcaaccagccacccttcagcccagcattgactatttagaaaggaccaatacactcactcttccaattgagctttataatttattattcctgcattaatggtctagtaccacaagatagctatttctcacagtacacttccacaggctatcagttccaccacccttcaatgcacaaatgaagccatcaagctacccggcagtgacgttggcacaggttgctgctttttaaggaaacaagagcttatccctggcgccaggccagaagtgatgattcagccagccccctctttccctggcagggaggagcagcaagcaaacaatccatgactaaggttttgtgcaatcagggctgacagctggaagtgtggtctcagtagtagtgttccttcctttctgaccctatctgggttcctgctgtcactttcccaaatatagtggccagatgctgatgtgaagccaaaagctggtagcaagacccgcagagaactctgaactcccctacaatgttcttgccctggcccccacctcatctgctggcagagccaaacaagagtcctgcctgggccgtagctgctcagaaatcaggcactggcctgaacagagcactttcactcacatcacgaggatgcacacaaagcacctagacacccaagaggtatcacaatcccaatttagagaggggaaaactggctcaacaggaagtcacaggctttcccaatgtctcagcactatgtgacagtaataatgaggctttgtcagagcccttgcttagtaacaca

SEQ ID NO: 16 - DNA, 1370 bp, Cricetulus griseus
cccctcctctgtgttttttgtttttcttggagccactgttatgtacccatgctcattctaacctccatacattctctcctttttataaacacatgggggcttaacaaccttcagtgactgcccatgcttcttggtaagctggcctccaaggggctcaggaacctttaggggatggatttactgctggggcccatgtaaggatcctggtttgactgcaaaggtagctagtttgattcagccttaagcttgacctaattcttcagagaaagcgtccactaggcattcctacattacacataccagctttcccaacctgtaagggggggttgtaggtcgtggttgggacgccaagtctcctgagggcaatatagcctaggcggcttagtgaggttgaaactagggtctcgtctgtgtgccaccagagaagcgcaaatccagcccagagtggaggactggcagttcccaccataggcacctcccttagtagtttcgtgctctacgctcctcctctggctcctcccatctcccttcctgatcgtccacgtccttccttatccaccagtcccattcttccagtcccaccctctccctctagtccctcctccctccaccctcctccccgcctccctctcaggctccaggtctccggcaaaacccggggcagatcttaggcccgaatacgacttgcgcacgcgcaggagctgcgccggctgggctggtggttcggaggttcctggctgctccgtcccgccgccctccaggcctcggctcctggttgggaccgccgcgttgagtaagtgcccggctggggccccgcactgggttcctggccggagggactggctgggtgattagcccggtctgcacctgtccgggtcttcgcgtctgctcatatcccctgcttgccgggtcctctgcttgaccaggtccccgtgtctgcccaggtcccctgcccagccggcgacagcctgctaccctgtggctgcgccccacttgcacctgcggtgaggtgtgtgcaccccaaggatctgcgtacacttgtccgggatggtctgcacctgcagcctcctggcgggccgggcagtctttggctctcggtccctcctcttttccgttctggagcctggcttgggctgcactgtgcgccacgcccctcgcctacaatgctgcacctggaactgggtgcaccatcccttagcggcccagtctagcggcccagtgcctccaacccagcccgactgggcagtttccctggaatgaaatctttgcgctgtaaaactccagtttgcctccttgcttgacagcttgggcggagaaagtgtcacacagcactaggcctttgatcttgcctttctgttgtagcctctctt

SEQ ID NO: 17 - DNA, 2683 bp, Cricetulus griseus
  modified_base (168)..(671): a, c, t, g, unknown or other
ctgtatgcagctgccaaacacggtcctgggacagactttatgggatggccacagtgatggaaggtcagaggcccaggaacgcaacgggttccagtgacagccaacggagcacagagtccaggcctgggctagatgacaggaagggaagggggcgttccggattaggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaccctaccagctgtcacccattgtggcaccgatgctggtggcgcccaggcgtgaccccgcccccgccggccgtcacccagccctgtggcgataatcgattgtctgtaggggcagcgccgccgctcctggggctacgaaggggaagagtgggcaccacgcctggttccctgacctgcatagtaggggcctaggcatcccccacgaagggagactgggttgagggcctgtctcctctggtccagggccagcatctccttcctaacctgaccagaaggccacgtgctgggggtccagggtgaggctgtagttatgacaggccaggaactgggtgaggggtggggtcctccagtccccagagcagcaccatgggggagggggggctctgcttacctcacttcatgcccatgcacctctgtgcttctctctcccacacataaattcatgctcgctagtccttggtggggcccattccaagacctgtaacttccaaggctctctatctgagtcatcccaatgcccagcattggtcctccaacccttgggtacaaccaggcttgtggccttctagcccgattcccacctttctaagagtgccggactgctgagaagctggcctgggtacctaccctgtgaaaaaccaaaggagctgagggttgtgaaagcagagtggtgtggaagtgggtgacccaaggggctgtccccggtggcttgagctaggccctgtttgaaggaagaaaaaaggcccagttctgacaccagaggccttggcaagccttggatggaggttcacacctagcattggtaccgagtggacccccagagtactagaccagagtgcagtgggcaaagcaaaagcagctggcagatgggatgagctggagtctcctggcggggacatgccccgcccaagactgatgaggctggttgggaggggctcccccaggagccttcctagagaactgcctccccaagctcatctcagcaggccagacaaagcctgctggggacacacgctgcctggccattgtgtaagggagatggaaggggccagaggctaggaaggcagggactgaggcatcccctaggcctcaggaaggtcctccaagtcgcttgtcctgggtacctccttgccgggatggagaccaccccccttccttcaggcctacatctgcccatcttctgaggtcagttaggagacagcctcacaccagccctagccccaggccataaggaagagatcactgggtcaaagcagatggagggagccccagaagggtgccagagttcgggtgtttgggctaggccccagggccctcgggaagcagcacacagtgccttcagtgcccagtgtggttggttgtacagggcttaaccaggtctctttcttactccactcggggtgaggggtcagatcagcagtctgtgtggaagaaggttgtctctctgccctttgcacccacacgcctttttggggcagtgatgtagattctgtcggctgtgcctactcagaattctcaggatggaaaaacctcaagctcctcattctcacgaagaggtgggcacagctgtagcatatggcgtcacatccctaaggaatatttgttgagcctcagggactagaaaaagagggggacagagccccctccccttcctcggggcttctccagggacacacaggttatgatggcgtgatactcagacagattgtgaataggaagtaagacctggacttccccagagaatgtaccttcccagtggacaagttgagaggcggtctaagaccaactagctctgtttggcattggggtgtggcccaggctaaggctgtctgtggtcttccagagatgccctggactggagataccacctgtctgggtggatgctgggctgagagctgtggctgtagcagttggagcagggaaggtgggtgagcacagcctggaagcttccttcagtcaagcaggagccagtagccactttctgggagccagaactgggaggctgctcttta

SEQ ID NO: 18 - DNA, 1262 bp, Cricetulus griseus
atactataaaggcaagaaatgagagatcccgtggggatatggggaggatttttaagaaggcaaagaagaatgagcgtgaacggttatccatcgcctccacggggttaagaaagcctgttggttgtggtgtcctggggttgggggtggagtgggaacgggtgtcaaaactgcagcgctaaccctgagaaacaggactcggagcgctgtggcaagaaagcactagcgaagctaatctataatcgtcctaagtgaacggcacacacttccctcctccaagaacgaaggcacgtctcccagtccagacccccaaacggaccccggtcctccgagcgaaggtctgggtgaggggcagaatggagagcaagggtgagtccctggcgccgcaggcagccagccacacagcccgtccaaaagccacccaccgtgggggcacgctggacaggtccaccagccccgagcagtgcccacaagccccgacaagccccggcagagcagctcggtcctcgctccctcccgcctgctctccccgcgtccggggcagcaccgggcggtcggctcggggggtctctagacccccttcctccgcgggcgaaaacccgctccgccacccagcaggaccttcccggaatcccactcccctcacctggcgccagccctgccccagaagggatgaaggagacggcggccgcagcaggtgcttcccacagggtccgggagcggcggtggctgcttcgcaccgcggccctaatctaccgccaacgccatgttggacggtgaggtcacccctgcgcgcttccgtcacggcccgcgtgggggagcggggctggggcgcggcgccgagccggcagtccgaggctcggtccgctggagcggaagcggtgaccctgctgcccgctggtcagtccgcgcaggcgtgacctgcgtacggagctgtccctcggcctctctttgcaggagccgggggagaaatagattcctggacttcagaaatgccttcgctaagtcatttccggtgccgagtagcgctaagatgaggtagacaccgcccgcctgctcagcccggtgaggaacgagcggctggtggcgtcgcagacctgtgcattccgggagtgcccaagcccgcgcgggttacctgtcagtacagcaatcgcgggcgctgccgggttcgggctctcagccacctacctgccaggaggttgtcctgaaagttggaaaagcaacttaaaagacaggactgacagacgggtctagagcccagacac

SEQ ID NO: 19 - DNA, 1013 bp, Cricetulus griseus
  modified_base (536)..(860): a, c, t, g, unknown or other
gttccatagggacccgtcaaaaaccatcaagatcctcagaaaagtgcctagattcgcttcccgttcaagagatgtaaagcagcataccctaacggttttcctgtcatttttagctccttcctttacagtgtcttggcagttttctatatgggaggccaatgctaaactccctgctttcaaagtttagaaaagggctttgcttctgtcctagtgaggactgaaaatcggtgactacagaccatctttattctccatcggtaaagcgggtagatagacctattttgcggtgcgtgcctgtggttcgcctctaccatcccaggttgagtcagggggatggcccagagttcaaggccacctaggggctacagtgtgcgggccctatgtcaaaaacaaaaacccagtccatcttgcaaagctgttcgaagatcatcggtgttacaatgctaaccctgtggatctccaaggtaacatgtcgtcggtaggaaagagcttcctttaccctcccggacacctttggccttcaggaggcctggccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccaccctgctccccttgctcggctccctcagctcccccttctctctctcattgtgaaagatcggttccccgactcggcacttggctccctgtggatggataacacccaggacctccaagttcaggctctctgataaaaccaggtgtccgagg

SEQ ID NO: 20 - DNA, 674 bp, Cricetulus griseus
ctgagccccctcccctcctgtgttcccatatctggatcgtggtccttggggtttcagatctgtccccatctccttcccttcactgtcccttccctacaataactcaggccgtgctggcccagcaggtgagggacagcaggagtcgttagccgaggattaggcacaacccgcggtcccaaggggaccagactcttcaactctaaaccgctccaggatactaatccgtatggctggcggcgcagcaagtcaaatgcagctgggctctccagaaagagcagattgcaggggtccgtgggtgcgggcgccctctggaggccgagcggctctcaagctcagggaagaagtctcgggctccccctgtcgggcagcgcctgctctgcactcccagggtttgctcgttaagtctgcgcctgcagtgtgcatacacaggtctgtgcctgggggcttactcgttcgtgcaaccaaggtagtggggcttctcagcctccgctcttgtggcgtagtcagaggacacagatgggacgaggactctgtctcgccaggtgtggacagcaggattcaggctggattctctctccaggctggcctcacacactcggggcttcatctgacttcgccttccctaggccccaggtcgaccttttttctgcttccttcttcagactgtctctagc

SEQ ID NO: 21 - DNA, 472 bp, Cricetulus griseus
cgtcattcctgggtcccattcgaaatccattcagaaacgtctccccagactggttcagggctggcatcccacacccaccccagtcctgaagacctttgctgtagctaggaatcccaaatttaaggtgtagctcaacctttgaccactagaggtctctgggaaagcattgacccgtataaccctgtttatcatcctaaccgtagacttgttgcacagccgggcgagcccgctctgctggaggccgccttctgacgatcgccaaccagccctggctcgtcgtggcgccagtcttgaagggggatgggccaggcggtggctgaccatcacacggggctgacttttagagtcacgattcaatccacacagtctaggggttacctctgaatccccacttcttggccaagcaagtagggggagagccccaggtgggcagctggagccctctgaatgtcaccctggccagaactaacca

SEQ ID NO: 22 - DNA, 418 bp, Cricetulus griseus
gtgcccatctggccaactttttttttttttttttttttttttttttttttttttttttttgaattttcgagatcagatttctctgggtagccctgactgtactgggacgcattctgaagctattgctcacttccatctttgtggcctttgtttcctcaccaaaaaccgagggcaggaagcagtaagccccttgctcctgtaagctgtccagacatctaccttactgtgcctggatttcagggtcttcccactagcctggggattcctgggaagacagatcttgagacagacagcagtagcggactggcacagctcctgcctgccaaccagggtttgctgagtaacttgaagaagcgcctgacacagcgctaggctagtgtccttaggggtcagggtgacccgtcaaacccagcagggc

SEQ ID NO: 23 - DNA, 911 bp, Cricetulus griseus
  modified_base (402)..(434): a, c, t, g, unknown or other
gtagactttgggagcccattccacatagaggaatactccctgagccaagacacacgtgggggggggctatcccaaaggatatgatagactctgatgacaccctatggaaggcctcaccctccctggggagcagaaaggatatgtgataggtagggttttagttgggggggggggtagtaggggaggacgggagaagggaactgggattgtcatgtaaaacaattttgtttctaattcaaattaaaaaaaaatctgaaagaaaaaaaaagaagaaaaacagcagagaatttaagaacatagaagggcgccttcttccagtcttcaggcgcatgtttttctctgcaaagaggccaagtggaggaaactggagaaaactggagactgaaggacagacagcgttgnnnnnnnnnnnnnnnnnnnnnnnnnnnnnnnnncgcctgcgcggtgaggctctctccagcccgccccgcggcctctgcgcctgcgcgcgatcgcccggcagaccccggggaggaggtgccggagttgtaggtcttgagccttccggcctggggcgggtcggcttcgtgcatctgctgggaggatcctgcgagaaatctggcctcgctgttgcattccggacagtggggtctgccccgagagtgggacgcagtctctgatgagtgtcccaccttaagacttgtcacggtgtgtgtgtgtgtgtgcgcgcgtgccctccagtgtgtgagtgtgttgggtagggaaagggggatgttaaggaacagaagaccaaaggactgagcagttaggggggagaagtatgttgtacagacatgttgcctgtgcacgataacttgagcagcctgccatccagtaactttaataatgaaagcataaggcagacgctttgcatttttgccctatgccctttc

SEQ ID NO: 24 - DNA, 419 bp, Cricetulus griseus
ttcagtgggacatctgaagaagttttaacataaccacaactgcaaagatcccatttctgtgtcaggttacactctcggatgctcagggttaagacagcaacataagaattttgggtgataagcagctcaacctacagcaagtggctttagtcagcaaagctaaacactaaaaatagcccaaatgcccttcagccagggaatgaatgaacagacatggtatttttatacaacaggatattactcagaaacacaagcaaatgatttactgatgcacacagcaccctggatggagctcaagtgccttgtgttacatggaagtaactggactggacaattacactctccaatggtgcagaagctgctacttacactgtatacgagggaagcacacagcacagaagacagatcagtatgtccag

SEQ ID NO: 25 - DNA, 380 bp, Cricetulus griseus
ggtgcgcacaaatgtcaggttgtgcttatggaagtcagaggacagtgatagggaatgtactgtgtatgtttatcttattgattgttaaataaaatactgtttggccaatgagacagcaagtagatgggactaggagtcaaagaggattctgggaaatgtagtagagaagtggcaatccaggcaggaagtgacatagcaaggggactcatttaagcgaaggagaaacaggaagggtctctcttttcccctctgctcctgctccagcggcaagatgcgatccaccgacaaggagggacgccaataaggtgtccgataagataagtcttataaaatatagagatttatgataattaagactgagcaaacagatgagaaatcct

SEQ ID NO: 26 - DNA, 534 bp, Cricetulus griseus
  modified_base (1)..(389): a, c, t, g, unknown or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttctccgctgctgttgctggggtttgggtagcgacaaggtgttcgtccaagcccggcctcccggacgctggcacgaccggacggggacgggaaacccgagtcccctgccagccgggaacacgcagcgagaccccgttccccggtc

SEQ ID NO: 27 - DNA, 541 bp, Cricetulus griseus
gctctcgctggagcactgaacaccccactggagggcagacagggcctgtgggacttcctggaggttccctcagagcacctgtgggtgacatgggggtacagggaggtgttcatgtctagcttgtctggttctttccagagaccaggctgatgggctaaggaggtgcagcatttattcggcaagaacagtttgaggactgagcctctgcactgagccgctttgctctgacttctcctgagcaggcacatggaccctgatagaacaggaagcaaatggttgcttactcagaagacagttgttagacagttgttttcaagggagtgggagggaagggagtaggagtcccaggctgtgagtcaccagtgagggcctcagtgtgtgtctgggattgactgtgtctctgaaaattcatatgctccagcagcttagaacatatccatattctgtgacagtgtcatcacagctgtaactacctaggacaaggtcttgtggagtgaggtgagcctccaatccaatgtgattgatgacattagaaaaagga

SEQ ID NO: 28 - DNA, 797 bp, Cricetulus griseus
acccttgttccctctcttctaggtcgtaccatagtgggacaggaatccccttaggaaaccccatacacggtgggtcaagggtgcttctccaacccacaccttcctccacaggcgttgagggccctgaaatcatcctgccttgcttctaccaaaggaaaagctgagaccagagaacatggatgcagggcaaagcccacaatccccttgcttgtgtcctgcgcggccataggcatttcagagttgttccacgggagagggcgacccctggtggtgatgagaagaatgacggcctgtcttacaggacccagaggaagtaggtcatccgaagctgcggtcttatttatcaagaatgtaaacagaggagagtcccttcgcggggaaaaaaaatctctctcctcactcttggtcctcagtcatgtttcccgctactgatttagtaaaacttcagggttttaattgaaccctgaagattggatcttcaggaagcaaacacagtcattatcaaatgatttttttggcaactagcaagccacgcaagcttttacgattcaggaatcgggtgctaagccctatcagagaacgcagaagcttgcattctaataagacttctaggatcaccatgacctcagggtcccaagcgagcaccgggccgtgcttccacggtagcccgctaagcggcgccccagcccaaaatcgagctcaggttcttattcttcagctcagaggaatgccttcttgtaccaaacgataaatgtctgcctccaaacctaaggaaaatgtttctttagccaagcatg

SEQ ID NO: 29 - DNA, 686 bp, Cricetulus griseus
ggcccggaggcatcgttttgctgtcttgggagcggccccggggtcccgcgcggttcgctgtgtgccgctcgctctccagcgcagtggctcgggcaaacaaatttttcagttccgcctcagggagtttttctgcctgagaggtccttggcctcgccgtcgtcttttttaaacacacaagttgtttggctggttgcagcggtgttggatttaatggcgacttgaacgctgagtgttttttaattggctttgatcgagagcagtgttgctgcttaacggtactttcagtggagagcagttactcagttctcacaacttaattattgaggtggtttaatggtacactaaaggaagtaaaaaccctaagcctaaagaggcgcattttaaaccttttattggttgccatggctgtgggtgcttgttacgtacatgcagtgtctagacgcctgcgtaacaattgctttagggttgtttaaccattaagctgctaggcatttaaaaaagaaaataagtctcaaacctaagcgccttaattgtatttgagggtattccagggggtattttgatgcggactcgttaatacttttctcccactttgagaaactgtggccctctgttattttggcaggtgttttgtagaatattttttgttgttttggaggcaaatgtttgtaaccctggctactata

SEQ ID NO: 30 - DNA, 471 bp, Cricetulus griseus
cataaatgcagtttgacatcttcagacctgcgcgctcgagcacacacacacacacacacacacacacacaccaccatcatcatcatcatttagttatatgtgtctgtgtacatgtacatatatgttggctcagtggataggctcaagtacactggctgcctccatccagagggctccgccctgataccagagtcacacagctgtctgagatgtcacaggaatttccccactgctctactcaggaagcctatctaccccttctggagctcgtacagtgctctgtttgcaaaaccctgtctccaacaagccatgtcatgtaaatacaactttgaggcttaaaaagacagtgtcacaccaggaggcaaagatgttgagatctctacatcctgtctcaggcacaagggttccgtccagtttgggctgtaatcttctcttactgtggctgtggcatgggcttgtagtcccagctac

SEQ ID NO: 31 - DNA, 483 bp, Cricetulus griseus
ctactgagccatccctccagcatccttgttgagaattttaaaataaaacataattgtatctgaaagtgaacaagctttgagaggaagtctgtggaggcaaagacaagagcctcacctagactgctggctgctgagggccccgaaaaaagtgttagcggagaggcccacgagctctgctctccctgagcctctgcagtgtggcatctcagaaggcaatagatccgtcagtcttggaagagacacttacttctgtgcacagggagaggcaagtcccaccagcagaggtcactgacgtgccagctgggaatccgcagctcctctcacatgtgcgcgtgcgcctgcctttctcccttctcagcactttgaagggaatcagtgcaaagtgtatgcttgctgttggcagatattctcagtggggcctgcctttgataccctcaaccttaggctgtttgttttaacatcgatgtaatacttagggcagct

SEQ ID NO: 32 - DNA, 382 bp, Cricetulus griseus
aaatcagaaatggagtaagttgtcagatgactaaagatgatagcataagcaacatgctcagcagagtcaaggctcaaaggcagagtgtagctcatggtcgcaaacaattgtaaactacagccagatcacaagactctccccacggccacccagcaacccagcctaggatgctgggagtaacactggtgagtaaccctaccagcacatcctgtactctccgacccgctaccatggaaatggagtaaacaggtcagctggaagagataagcagtgcccagcccacagtgatttgcttgaaggaagaaagttcctttatagctgagctcttagaagtgggtgcttccaagctgctgatccttgtcttcaagcccaaagaatataa

SEQ ID NO: 33 - DNA, 958 bp, Cricetulus griseus
  modified_base (304)..(801): a, c, t, g, unknown or other
cgcacacacacactggggtggtgggccccctgggtcgcagacggtggttctctgtattatgggcttcagtacgcttaggcgcgcttgctcatgtcacaccttaaccggcacccccgcagccttcggaacaggccagaacttctaatcccgtcaccaccccgacatgtcatcccccagatccttggctctctggcgttctgaccgctcaggggtctcaggtggaggctgcgagggtgctctctcccgtgtccccgcagggctggcactcggggcggtggaggaggcgcaggaggcggggggtggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctcgggcctcccccacagccctggcccgggataacgggccggggcatgggaacgtgggagacgccaccagcccgcgacaccccgccacatcctgtccccatccaaggggagagggtgcacccggggccaggctggcatctcataggcccaaaccca

SEQ ID NO: 34 - DNA, 509 bp, Cricetulus griseus
tggaggaggtgagcccagcattgtggttggaaggaatgtgggcaggcctggcaggaaaaggagttagcagagagcatcctgagaccaggaggccaccacaggttctcaggggacaatgggtggaagtggagtgaaaggtgtcaggcctgagctcgccttccatggagagcagacgccagggcagtgaggccccttcccttgtatcaggatgccctccccagaaagtcttccataacagagagagtgagcatgcttccaggaaacacacagtgctctcccccagcttgtgtgcactggtctataaatacacccagacaggcttggctggagcctccataccccgagggcatctggatctggctctgagctgcctttgtcccagcagcagtttcgtcacccagcagactgggggtgggggccgggaatgcagctgtcacatcctgagcccaccgaggacacccaagcaagcatttggcctgtaaaggccaaactgaaggccaagttgactc

SEQ ID NO: 35 - DNA, 463 bp, Cricetulus griseus
agacagaggtggtggctgtgcaatattgtgaaagtggtaaccaccatgggtttgtttgctttgcattttattataaccgaaacgagtaagacagaaccctatgatggctctttgagaaaggtcacacttctgagcctgccattctctgcgaacctggctccaagctctttgcaggttctctggacccttctgtctccacaggttctctcctccctttcccttctatcgccccctagtggcttagaggcaagtcccaccactagggctaagccatcttctcagacctccctttttggtgggagtcatcaagagatgtggaaggcagagaactaaagtttggagtgaaaaagaactggattcagcttctcattgttctacttagctgtgtgatgtagagcaaggtcagtgcttcatagccccgtctcctgacacagctgtgacagccacatggcaggaagcactg

SEQ ID NO: 36 - DNA, 476 bp, Cricetulus griseus
cctagtttctagctgtggagcagaaactgtaggtgccatcttggcagaagggaaaggtgcacctccctgtaccagctgactgagagtcatgtgtggttggtcttggaatttgccccaggggataaacacagaccctggaacaatcaggaagaatctgctgaacgctttccttgtgcccagagggttccaagccactggccagagctagacaggaaatatggctcactctggccaactatgagcctctaggggattgttcttcagtatctctgcccaagctcctcttgaccaagtctggcatgaagccttggtgctcaatccatgcttgcaggaggcagaggcctgccaagctctgactcctgtaccttctagggtgctgaaaacctctggttggtgtataggctgtgtccaggctgacctctgggtgaggatctgcaaggaatgactggcctaactcctcatttacatccaggctc

SEQ ID NO: 37 - DNA, 1172 bp, Cricetulus griseus
cctaaaatggagcattctcaggcgagaggggctggaggacagacacaggatgacctgtggggtctgaggaaatctttaacgaggcagtttaaaaaggagcgaacaggagcttgtgggtgagggtgagttcccccacactggacgccgtaggcaaggcgtctcatctccgtaggcacggacagactctgcaccctcctggcctagctggaccttcctgtggcccctcccacgctcccggggtcccagccccccccccaggggtcccggacgtcgcagcggggccctcccgcagccgaccgcggcccacccgatgtccaagcagccacccttgtcagaggctgacgactgcgctccggccggcctggcctgttcacgcgccctggccaggccggcgggctcacgacacagccccctctcccgggcgcacagacacggcctgggcctcactcaccgcagcggcgcgcgcccgcagacaaagccggcgccgagcccgtagatgctgcagcagcaacgagcggcgccggactacaactcccaggagcccgcgcgcgccccggggcactgtgggaagctccgctgcagagctgcggcagaaacgtggaaggctggcccggtgttggcaggttctaggggaggggagtgccgtcctgtggaggagctgcggttgaagtctggggatgccgaggacattgttgaactacgcaagactcagggttcactgtagggctcagaacccggaagggagaatttcggaaaaagaatcaggaacacccgctggtcggttcataagatttctcaccccaaagatggacgaggcaccttcaggcttcggaatttaggagccacttggactagttgtgtgatccagggtgagaatcccacctatgggggagttcagagtgtgggcgtgacgaccgatcggttggacagggactctggcactcaggagtcaaagaataccacaggtcacagcaagcatagcaactcacagccctgcacattttgtttagagtgggatgccttgctgtgcctgtagaggtcagaaaacagtttgttgttgtggaatgttttaaggtgtgttacatttgcttatgctgtggaacatttgttttaatgatcgaaggatgtattatattcatttatgttacatttttttttttttaactctgcga

SEQ ID NO: 38 - DNA, 1221 bp, Cricetulus griseus
agaagtgggaacctggtttaggcttctcagagagtgctttaacttggcttttacaaacaagtaaataaaaataaataaagcaaaaccgatcttcacgacaaaaatggagtaagatgcgaaagtgggaggtttctttggagaggttgccctgttggtgggctgggacgacctctctgcgttaaccgtatgcacagaggctatcttaggccaaagtcattgataccaccctaaccgaattcagggattccctgtaaaattcacggaagtaggaaacggtaaggatccactccacggtggcggtttttcagagtattaacgagaccataatgcaggcttttactcagatccttcatggtgaaggagatcaagaaggctaaaacgcaaaatcctccttctggcctccccgtcggggaatcgaaccccggtctcccgcgtgacaggcggggatactcaccactatactaacgaggatgaaccacgctggttctttcccagacagattgctatagacggatacacgcagcggctgtcacccggttcctttctttccgtgttcctcctctcctgctttttctctctcttttcttctgctcttttgttctctcatctgtttctttccctctgacacctcatcttatgacgcccacattatgatcagaggccaaagacttcacaacagtgtgtgttgataatcagtactattctggaaaactgtctttacagcaaggggatgtagctcagtggtagagcgcatgctttgcatgtatgaggccccgggttcaatccccggcatctccagcttttcttctttttaccccctccagtaaattaggccagaatgcaacactacagacccaggcttcctttcccaccacgtggagacggccctatggggcagactgactgcgcggatggcgggaatcagcatttgtaagtcaacctcctgtagtaacttgtgagtggggcaggaggaaaacattgcatttccaaggaggttctataataatcggcggctcctgcgcgctcattgcctcgtaatgtgcacccgttgtgcacccatcgtgcgtccaaatagatcaagtagcccagtaaccttgccaagaccacgtaaggagtctcagcctttctggaactcaaattccgactcgaggctatcacttgaaatccaagggatattttatgaggacgcacagaactgctgggacatccacgagatccgca

SEQ ID NO: 39 - DNA, 480 bp, Cricetulus griseus
aataagatgacagacaacaaggacacagagaggtcaggtatggagcaaccctgactttggcatcaatcaaggtttgcttcctgtgggactaaggaagtcagaagtcagaagctggaatctacaaatggtcatgggcaccgacccaacaaacagggcacggctggctttctaaacaaccgaagcttctggaagcttctggagtggacctccttcccgctcgctcgcctctagtttcatccctggccgccaaagctcgcctaggccgagtgtgtgtgtggggggtgacgctgtggaacacaggctgcagagaccgtcctgtcctcaggccacttccacactccctcatcttcgtggcctcatcttgcatataagcatctgcccctgagccttctccatcctttcatggccagccaccgatttacttatgtccttcccaccgacagaaaagcaaacctgggtttttaacaagccatctggggt

SEQ ID NO: 40 - DNA, 1181 bp, Cricetulus griseus
gacacaggtctgcagcagcaggagacacaaatactgtaaacagcaggtgacttctctaaagacgcttacagtgtgggttttgggctcgaacttcctcaacccaaaggaaaaggaagaaaagaaatgatttttctttcttttaaacagctgtgtaacttgctggctaagcaaaatgcttttccttgttatagcttggaaaagagcagcacttggacctctctccccttagcccttcctcccaccctggactgtaccattaaatctgagataatggggtgatcaaagcagaaattaagttagcacttgtcaaaacctgtccctctgcatgcatggcctcgcctgttactttctcctgattgtggcgatcagaatctgttcaggggcttgctgcgcttgcaaggtcgctgcgagagggagcattgaactttggactgcacctcagagggtgaagtaaacaacagggctatatatgtcagtgtcattctggctttgtggagctgccctgccagagcaaagagagagaggaagcaaactagtccgaaggggttgcgtaagtaggacttgattgtttagagtttgagaaaagttttctgagactgttaaaatacgtccccgctttacaataggcttagtaaaaagattggtaaaaggcataaaaaatactctaaaaaatgaacaagagtttgggtgtggtaggtgaattgtctggggattgctgttagccttttttatttttttcttttctttttctgtgctgtgctcggggaagctggggaagtggactgcagctatgctatttctctctgcaggagactgtggtgttctatgggcaatattatctgattaaatttctgtgtttcccattgaagggtagagtgaggacaaacaaggggaaaagctcgtggatttcaagagcagagacagaaatatgcccattttacagtactgtagagatagatgtcctgggaaacagtgagcagcttcagaatgacgagcagggaggggacaggctccatccacagtgcagaagtggtcacctttagaggaggtccggctgtgccacaggtggtacatgggtagtttttgagttaagcagagaatggaagcccttaggtgggtgaggggctttaaagtgtggttcgaagttcagagggaggggctgcgtgacaaagagtaagtat

SEQ ID NO: 41 - DNA, 719 bp, Cricetulus griseus
ctggtcattggcagtcatgggaaatccaggaggctggaggcagtggacaatccactgtggtgttaggaagacagcatagcttgacatacaagctcacacgggcccaaccataaacaacatgcttggtagttacgaggacacataccaacattccagagcccaggaagtataaacatggagaaccaggctctatctagcagtggccagagtcctggaggacagggaggagcaagggcctggaggactgactgtgtccctgtccctaagactttggaagctaaaggtttgaccacaggacactagccctcccaagtgggaggcaggatcttctagggacttcagcagaggatggatggggctttccgcagccaccttcgggaattgtagacacaagggactaggaaataaggaatcagaaaagcccagagccagtgacgacaccacagctaagggcaaggaacgaaaagtggaaaatgtgagtggggagaaaggaatggagctagcaaaaaggggggtggggtgggaagaggatacaggagacagaattatgggatggaaggatgagtgcagcaccctgctattcccttcagggatggagcctgggttttaactagactggctggccaacaagccccagggatccatctgtctctgtcagtctaccccacacccctcccttgcctgccctgacatgtggctagcacctggctttttaggtg

SEQ ID NO: 42 - DNA, 864 bp, Cricetulus griseus
agtgtgcaaggaggattactgctacatggcacaaatgatttggaggttagaagtgcgtgattgcctcacacaaggtcaaattcaagatcactttgctctatataaagtttagcctgacagcttgtcaacctcaaccgggcagggggcaggtgttttaggaccttgtaacacttgttacacaagacgcagcccaactctcctctatcacttcaggcaggaccttgatcagagcccgggtgtggaggcagtcaccccgcgatcctggccgctcctgagcaagtattcatcagtctcgagcccccataatcagcacaatcgcagaccgaagagtatcctcaccttccaggtgcccaagcccacaacagggatggtttgcatggcttcccttgacccactggctcagcctccaaacagcaccggagggcccagagagcagctgcgagtgcccaactacgtagaaattccaggaagcgtaatggcgtgattgactggagttctggcttgccaattggctggcggagaggtccgaacgttctctgattggctagtagtatgatcaatctttcagctgagcaactgagtctggttgtgcctttcacctctcataaatcctctggaatcctaggagtcctgtctcctagcaacctggctaaagctcgcttctaatacttggtatttccagaaagtaggctggctggcatacaggtttatttcctagaaatcagattcgttgtgctgatgttcgttcctagtatactggaaaataattgttatggataaagtaactattcgtcttggatccttgctatggttgtgtgttaatttgaggagcatttgttgttttgattgttttaaaactataga

SEQ ID NO: 43 - DNA, 776 bp, Cricetulus griseus
  modified_base (358)..(697): a, c, t, g, unknown or other
cagggaccgattttttttagggtcccgctggactaggggcgggcaaggcctccaagaggtgatcgggtgtctattgggtcccaagtgaccaagtcgctcccttgctgcaagcgggactagaaaccagtgcagcaagggaagtccgaactggccgcgttggccccagcagaggctccctggaggcggggcgggggccatcctcacccaaaatcgtcgtccatagacttgaagaagaacttatagctgggccgctgcagaacgcccttaaagtccgccaaggtgacgcgctcggcgggcaggggcagcttcaccaggtacggcgtctcctggccgtccaggtggtagatgatcttggtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttgaagaccggaggaggatggcttcggcgtgggtctttgggttctgctgacaacacagttgctgtcacaggacggtac

SEQ ID NO: 44 - DNA, 1442 bp, Cricetulus griseus
aaggtcagggatttctaggaggtaattccttttttttttttattagttcaaattaggaacaagcttgcttcagatgtcaatcccttctccttctccttcccctgccccccagtatcctctaggaggtaattttaattcaatctccacactccttgcagacacccgcctttctcctcctgagttcccattggctcctgaggtcagccactctccacgaccagcgcggttgctggcccagcctggcttcacaactctcaagtacctagtcgctctcaaaaccccgcccactccccaacgaagttcctcccacttgcccgcccacctgaaaatccccttccaccgtcttcgcgcgcgacggagttcactttctcccatctgtttgtcgaagctccgccccctctaccgctcgctcgctcgcgagcctccgcccacttcaccggcccgcaaagccccgcccccttctgcccgtaccccaactccttcccttcttccgtcgtttctacctttccccgcccccacagctgtctttcattggctaaaaaggctaaaccgcgcggggatgcagcagagccacattctgattggctaaaggctcagccgggccccttgtcattggttgatacgagaaaccagcaagagtggctgtgctgggggtgtgcggccgctgctttgttgccggagggggcggcgttggaagttgcagggccagcgttctcgggaagtggagccgcggccgctgctatctttccctcggtagtcttcggccagtccccgcccgacaacagccatgagcggcggcgtgtacggcggaggtgagtgagcgcggcagtgcctcggggcatgcgggccggaacgaccggcccggtccctggccgcttccgtttccctccggtccccggcgggcccttccggtctttggccggttcttccggtccccgttcgggtcagggcctctccgcctcttcggacccggagcccgcgtgcagtgggctctcggagatggcctcgagggaggaccttgccgcatcttgcccctggggctccaggaccggcgggactctgatggcagtcacctggcttgcggggtccctttccgtaccggttcctccgcgtgttcgtcctccgcagctcgcggaggtttcgggccctcggtgtgggctctgggtgcgagtcgcacccggtgttgacgctgctgctcccgatggtgcagcagctgctcccgactagatctggttcctggtgtgtccggtctctagatgagcggagcgctgaggtacgcgctgctgttcccgtccaaatgggcaggagccttttatagattggtggtaacgaaatcaaggctcccaagtacttgtttattcacgtgtaactagttagggactcttctctgcttggatgctgacagaagatggggccgctgtcccccgcgttaatgaagaaag

SEQ ID NO: 45 - DNA, 478 bp, Cricetulus griseus
atacaagactggtgagcagagggagaacttgggctgtggctgcagttttggtatagggtttcacatagcctacaacttaccatgtagctgtggctgccatctccatgctcagaaggagcaagaggctcaggtgcctcagccccacactgaggaatgcagacatggagggcctgtggggtagtcaagttttaaagactgtttggtaggttgaccatgctccagtgggtggctccactgagcagtttaggggtagcacaaaacagaccagatcaagttgaagctactggtttggatgaatggtcagagcatactgtacaaactgccctcccccactaaaccaaacaccatgttcaggccctaatataggtttgagatttgagaaaccagttgggcagtgaaagcatgtctataatcccagcacttgggggcaggcgaatcaaggacaacctggtccacacagataaaccccaacacgaaa

SEQ ID NO: 46 - DNA, 2038 bp, Cricetulus griseus
ctgtgagttggttgagcacttaagcacaaatataatacttaggtaaactaacaaattttaacaagttgttgtgtgtgcaaaaacgctattcctcatcaatgcataaatgtgttacttctacacgacagctacaccaagacaatgataaattaggcagcaggaaaaattttggtgaatctgcataaagctgcgagttttgttgacaactctggccagttttcttacagttgtaaagaacatactgtttccaaactatttcccacatttaataatctctttgagaacgaagaactgttctatttaaatgaccttactagcatccaatcaggacatgattttggaaaaaagacctaacaaagtagttcattctgagccaaacaggagtgaacatgaattcattcaagttaccctgaataaagtgttccggcatggctgcgttttaaatacaaagtggggaagggctgactctagctccatcggtagagggctaagtaagactcttgaggctctgggcttgatcctcagcatattataaaccaggtgtggtggtacaggaatgttagcacgaagaaggtggagactagaggatcaggggttcaaggtcaccctccgctacttatgtagttcaaagccaacctaggtttcatgggctcctgtctcaaaaggaaaacaaaaatgtatttcagtttgacatctcacacaaacacccacgcagactcctgggtcgctgagctggtcgggcacatatcctttcccccgacacagaccattttcacacgcgcctctgaacccttttttggcctaaattagggttgttcccgcgcttgcgtctatggccgcgcatgcgtagacggtaagagccctacctacctatgacgcacgattgacgcagccgagagctcagttcccagggcaacgagctccgactgcccgccttccgctggtctgcgtcacttccggttcgcctctttctgtttcatcctggttgatgattggttccgccttgcgctgtgtgcggcggggcggtgctgggaaaggcgagccttcggctgtggataggacctgcgctggggtgggcggagtcgggaggagcttggcttctcctggccaatcgctttcaaatctgccccctgggcgccgcggagcatgcgcagtctcagaaggcactataatccactcccggagtttgcaagcatccggagggtactctgacggcgggcgggaggtgtggccttgcgggcggggccgctggtaaaggcgttgattggccgactactggtgctggagtagggacttagctcattcattcattcattcattcattccttcattccttcattcaatggaccgagggacactaggctttacaatttcgaccctgcttcccaagagtccatggtttggctgacactttggtgtcgccctctcttttaagtctggtgccatagcgttttgcaaaagggggacatagcctctcttactccagggaccttctcaggtggctccacctctgaactcaacacaccccaaacctaagtgacactctggcttgctgcttatttaccaccgagcccttttccttcactttccatccacccggacagtagagctgtctcacagaggcactgtgaggtttgaaatgagcgtggatcagctaacctcggggttagattccaggctttttggtacactgctctcttagcatgcatgtagtcttgggttcagtccccacatccctatgatcctcaactgtgcagttgtaggcaaaagactcagaagatcaaggtcctcctggattacacagcaagtttcaaagccaacctgggctacatgagagctgtctcaaaatcttcttttatagctgttgactttattataagtgtaccattatgtgtacattgagtgcaggtgccacagaagtcagaagggtcagatcctctacagctgcgttacagacagttgtgagtcatctgacatgcgtgttgggcttggaactctggtcctctgcttgaagagtatttgctcttaactactgaac

SEQ ID NO: 47 - DNA, 404 bp, Cricetulus griseus
ggacacgcccacacacatcctggttgtccacactgttgggtagtggcccgggtagaataagaatggctggccttaaccttccctgagtggtgtgaatgtgtgcctccatgaacagctcttagcactggatatgctgtcgtttgcctggcaaatcagagacagttctcgcctttgtcttaggactaatttctgcagtgaccttgttctcaggcacttcagctaacttggttggtatgtgtttttattagtcatctcttaacccatttttccctgcagtctagggactttggaaaatcatagctagaatatttctggcattcatctgtcccctgccctgttccagccctgggttttgcccatcacagcctcttactacactgaggccagccctccagcccagactg

SEQ ID NO: 48 - DNA, 917 bp, Cricetulus griseus
cactggagtcctcatcccaggccaagaatggaatcaggaatagtcagatctattcttcactgtacaaaacactgccacacacaatatttcaagagcccatcaaatgctgagttccagtgcagaagatggcaaataagccaggcaggagacagaaggtgcttgcctcctggggcttggagggcgggccttgggagagaagccagactaatgaacactggggagaagaatgtctcccctcacccagctctggggcctcagctgcaagcacagtgcaggcctcaggcaagagcatcagagggaccttcagacctggagggcccagaagaggagtgtgagtgtgtgtgtgtgtgtgtgttctccatggcctctaggcccactgtccagttaatgtggtcaagcctcagcccctgggagagggtgtgtgtgtgtgtgtgtgtgtgtgtgtgtgtgtgtgtgcctggcccagccagataccttggtaccccatcgggccaaggccagcgtgtgtgtgtgtgtgtgtgtgtgtgtgtgtgtgtgtgtgtgtgtgtgtcccctctcaggccagcgtgccagctgctacagaaccctcaggctccaacattccaacctctcttaaaggggcagtccctgcttccaaactgcagcagggtagggaggggaagacaagagaacacaaataggcccttcctggccttccagccaggctctgaagccctatgcaacatctcctctgggatcaccatctggcgagaaaatgccccagcacctggttagtgaggactcctcctccgggtcagacaacaccaggtcagaccagggaagaggggagacagctcaataaacatcatgtccagcatctagcctggcagaacccccaccccccaccccccaactcctccagttcaaagtcccaccgg

SEQ ID NO: 49 - DNA, 458 bp, Cricetulus griseus
ctattatttcactgagttaattatggactcaccccaaaggtatgtgaatataccaagtgtggagctattgttgtctagtgtagaggaactacagcttgttttcttgcactttcagttaaaatgtccccacgttgggggacatggctggctgcgattcctatttgtagccacaagatgacaccattgctcagagtctttactctggaaaacagctcagcatcagaaaattctcaccaccacccggctcgagcgccttctcttctcttctttccatcttctctctttccctcttttcttcaataggcttaaacagcactacaatagcaagtctgcagctcctggaaggaagacacaaagcaggatactttgtctcaggcagtactttgctcaaggccggatccataaactttatcttccccctggcccaaagcccagagccctgctgttccaggcctaac

SEQ ID NO: 50 - DNA, 461 bp, Cricetulus griseus
tccccttcttcacacgagtgggttgcatcaatggacaagtgcgacagaaaataagaactcatgtcagtctcctaggaagcatggtaagcaagaggctaggagtgaaataaccttgttgaacaggacgtcatgctctagcaacaccactagagggtggaaaagactcaccacagacccgaattggcgcctctttcagaaagtgaaggctgctcacacattttctgagaacgaaggaactacagcaaagttaaactccgggtagaatatgtctataaatacacacaaacacatgtgcgcttctgaggactgaaggaaacgtaggtgcgcttgttgtgctgccatgagttataaaatcttgcaagcgtcttgttctagtcctctaaatcagggtgaagacatgaatatgcagagagaagaataatgggaaagcagaaacagcagtgactactgctgccaccaag

SEQ ID NO: 51 - DNA, 319 bp, Cricetulus griseus
aacttgcttttttttttcttttaacctcaaaattcaactgtgttgaatagggtagtcatggatcattcattttccctttgcatttcgtccagtctctccttgatttgcacccactgagaaatagaaagtagtctatttgcttgagataccaacaggcatccacacctatccagcacgcagcattggtgccccctggtgcacagtgcaggaattgctgcagctctaatgttacctctattgtaggagttgagatttactgcttaccatggcaagccatgcagaccggccagagccctcttgtgtaaaaccatacccaaaa

SEQ ID NO: 52 - DNA, 842 bp, Cricetulus griseus
  modified_base (94)..(203): a, c, t, g, unknown or other
gagcccgagtggcctcgcgctggccttccgagcgacgtgcggcctgctcaggagaagccagggccgtggggacttggggcggacacaggagccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcatcagccgtccggaactcacctgccgtcgcaaccgcgccgcctgtcagtcaagcgcgcccaccgcgacgccgcgcagagtcttgtggctctgcctgtcgtgccccgcctccctaccgtcagctgtccaatcgagagagcctatgccaaggacggggcggggtcacatggcgctgcctactagggggcgggaccgtgcgtgctgggggcgtggccgtgcgagtactgagtgcccatgcatgctgggagtagggggagtggccgtgctgctggggcgtggccacgtggttcgaagactgtcttgctggtggagggcacagtctcggcgctggggcgtggccacgcggctgtacctgcccttgctttttggagttgggagctggtggctatgctgctggggcgcggcctggacacccggaggcgggtctgcatgcagccggccctctgacgacacttctgcgcgccggaagagaaagaggttcccggcactacgcggcaggtaacgccgctcctaagatctggtgccgctatgcggatcactgggaagactttccgccggcggcgggctgactcggagtcggaggaagatgagcaggaatcagaggaggttcggtacgtgtttggcatgacctccacgg

SEQ ID NO: 53 - DNA, 958 bp, Cricetulus griseus
gtgttcgtccccttgtcgttctgtcgtgaatgaccgtccccgcgcgcctcgctgcacctagatcactgctgtgatgagtactgtgaccctggaatagaaccagctctctgagccgcagccccgcaccatccgcaccaccccgctgctgccacgcatcacagcggtctcggaatggggtctgggaggagggagcgccaacggtcaggcaagccctggcgcatgcttgtgtccccgggaggctttcaggttggcatgagcagcagggaaaatatacgactgctgttcccgcagatcccattccagaaaggcgagcttcaagtcgacattttaggagcttcctaggtcgcggccgcgggagatggcggcgggagtacctcttgcgtggagccacactgccatctcctggatttttcttcgcactgcagcagcagctgctctggcccgcgtgaacttcggcaataccggtgacccgaccagcgtcccactcccatgggcggagcaagggccctgtccccggggatcctcagggagccgggaatctactctgcagaaggggcaatgcccataaaagagggctgtttccgcagtctctttatcggcggcagacagagcaaacaaatgaaagggttttggtgggataccctaattggagctggacagttgtaaactcattaagagccgaattccagacagcagaccttcctttatggcacacccttactgcccaggctttgaagttctttctgtctgcaacaacagagggcgtgccaacctggcggcgcattgggggtgggggcgccccatccacccatgtcctggttccctttttcctcgttggaagaccgtcaacatttcagggggagggtgaagaaaaggagctggggaggagagcaaagcagaatcccatggcttcatctggaaggcgcttgagcctccaaagttgtagccggtcccccaa

SEQ ID NO: 54 - DNA, 462 bp, Cricetulus griseus
gtgaatttaaggccagcttggactacatgagaaaaccctgtacatacatacacatacacacatacacacatacacacatacacacatacatactaatattgatatatttctctagaatgatgggcaggagagccacaggactcccacaggagcagcggttttctctcccttgctccttcctctttccccctttcccatcaggccttgcaaatcccagtgtggcaggcagctgaaagcagaactttgcgtgactgctcggctccccgccaccgcagaagaagatcagaaacccaggcacagactctgtccctgaggaaagctgtctccgctcgtgacacatggcatgctcgctcttcagggcaggttaccatgtaggaagatcctttcccttctatttctatctttcttggtaaaagccatcatctgaggtgcagggtgaaacaagtggacacttgggcattt

SEQ ID NO: 55 - DNA, 740 bp, Cricetulus griseus
  modified_base (238)..(497): a, c, t, g, unknown or other
acagcgatcactatttagggaatatgtcaacagacagtgcgggaagaggccggagtatagcatgaaattactacctgcggtgtggagctgtgggatccggtactgaattcttacccactattggcagggtgtgaaaggaacaccccaggtctgggtttctccaacccagaccatgggaggtctgaggtctgagcctgggctgttcctgctccttcccggctgcggttccggcaggccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaaacctcagcaggaaagaggaaaattctctggaggggggaggggaggggggcagcaaggcatgggagggaccgaggccagaacagcagcagggtgggctgaacctggggtagtcggaggagggtgggacaaaggccagcccaggatgccagtatcatctttcctacatccactgaacttctaaaaccttagctagacctcccgagaactccttcaagtccttagcacaagatgtcccaatac

SEQ ID NO: 56 - DNA, 567 bp, Cricetulus griseus
ccacagcttagtgctaaggggtttacttagattatctcgctgaaaaccttaggtttgcagagtaagacataagtgacccagagatgcagagctgggactcggtgtcaggtggctgggctgctaagcttcagctcttcgtgtgttagcacactgtagcatccctaatgtctccaacactcaagctctgtaaccactaaacaacctgcaggagaggtgggaaggtgattccaaagcccaccctttcccctctctccactctgctctgcctgaggataatacacacactgccttgacttctaggtggacactagttgagttgaaggactgtaagcagctggacttggccaaaagagacattttgtgtgagccagtgctgccacaatcaaagaaagggttctgctgcccccttgtggtgcaggctgactgctactcagcaatgctccagtacaaagctgaaattccatataaaggcacttggccaccagacacaaatcagcaggaggccactctgattggctcagagtggaacagatttaataacttgaaaatgtgattttgtcactcttg

SEQ ID NO: 57 - DNA, 347 bp, Cricetulus griseus
agcaacacctaagattgtttgttttttgcagatttctaattttttaaattcctgtttcgtggtcgtcaagctacaactctaacaactcaaacaaaacataatgtgtgctagtgccaaagtctgcaggctgcattgtttcctaatctcatgtatcagtcattttacacatttaaatccccaccagtggagggtatgctgggtgactcagcccaaactgctgctgtgagaggagtttggcaaatggattagagttgtatacagtttcttaacaagaaagtatctttattccttattccgtgatatacggagaagactgatgaaacagatgctatttcttatataattaa

SEQ ID NO: 58 - DNA, 1007 bp, Cricetulus griseus
gaagagcatcagcttcgccgttccatagaaaacgttgcgagggtttttcagaggcagtgcttatgaaaagccagagccttaggcacccgagggcaagccacacacacacacttctctgaggtggacttccggcttccacagggcaaacactcggtttcccgcagagcaggaagtattccaggaccttggtggagccaagaagggtctgtttgctagcagagaacagtgcccgccacttaataggtgcttggtgcatgtgtgtggaccgaaatcattaagtctgtgaagaacgagccgcactcaccacgccgaagcttgccaggtctggccgctgcctggcgctttccaccgcctccagtccaggcactcggggagctagctacacaaggctcgcccgtccgagctggggtcctcccgggacagccagctgcgctgcgcacccaccgcacgcgccagagtcggaatggaaggcagagcaaaaacttttcccgccactagccgtaactccgcccacagcctgatcccgattggcgttctcaaccatgtcgccacgcctacccagggctcatgacgtatttccgccatcgctgatgcccgccctgcgtagttccggggaagtgttcagcgtcgcagtttccgcttccaagcgggttgctttcagctgttagaagggtttccccagcctctgaaggtctttttttaaacctggatgtgacgtattgaagaagccgacggaaatagaatcgaaagcgttctaaaatggcgaaccgtacggtcaaggatgcccacagcatccatggcaccaaccctcagtatctggtggagaagatcattcggactcgaatctatgaatcaaagtactggaaagaggaatgctttggacttacgggtgagtgacagagtgcggcccgcgtctcggccttgcggctggcttcttctaattcaatgaggaaggatagaccctcgggtgttggtactgcaaaagagtatgtcaaccattcacataa

SEQ ID NO: 59 - DNA, 777 bp, Cricetulus griseus
  modified_base (77)..(777): a, c, t, g, unknown or other
gtgtgtgtgtgtgtgtgtgtgtgtgtgtgtaggaggggggctggaaggggggggggagggggggtgtgggtgggtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 60 - DNA, 570 bp, Cricetulus griseus
  modified_base (1)..(292): a, c, t, g, unknown or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaatttataggcttggaaagtatgaagacagttcctccatctggagtgatctcagtgctgtgcttgcttccacccccgcctggatatgatctgggaagtctccagagggcgagaacctgcttagcctatagaatacaaagatgggtgagatcagcagagacacctgaagcacaaagcagaggcacaaaggaggaaaatggccactatgaagacatctctcagacagtggacacattttcaatacagactcagcagtacataccaaaggaaagtgtcta

SEQ ID NO: 61 - DNA, 403 bp, Cricetulus griseus
aacatgtcacgtgagagcacagaagaggatggtgacaactgtccagacacatgacccccaaaacaaaagacatgagggaggatgcaattgatttgaccctgaagagagttttactcttcaaacaaaacagggtcttctgctgctcctgaggccacttgccatgccactagcctggagccaccatctagttttgtgtagaactgggttcttgcaggtgccaccactcagagggccaccagagggtgctttcctcctttcaggttctgccttactgcaaacagctccttttgctcaatagcactgaatttcccaggcattggggattcggcagatgcagctgtggcagacagcttgggttctgggcaagagaaaatgtcaataattgtatgtgtgtatttcttca

SEQ ID NO: 62 - DNA, 1201 bp, Cricetulus griseus
ccccgcgccgccgctcggattcaagtcgcatgaacgggttctcgcttagcggacccagactctcggccagcaccagcttctcgtcctgcccctccggcgccgctccttgtcccgggcctccgctcagggcggccagtttagctcgcctccgctcaaggatctcccgcttccaggcaggcatcgcggtgcgcggccccgggcccggccgccctaggccggccatgctgcggacgcagccgccgacgggactgcacaggctgcggctcgctcagacagactctctcagctacctgcctcagtaccgcgccgtgccacgccccccttggtagccgccaattgggcaagtgtgctctgacccaagggcagaactgcccgccaatggtagaggtatacgcccagttgattggaggacacgcatttacaccctcccctatagcgttcttattggactacgaccggcaccgaccagtccctatgggtgaagatcagtggacacgctccgcacctcaatgtctgcccctcacttccgcctctgcgctctgaacccggagcctgaggaccacaatttggcagggctggtcttggcataacgcctagcagacccttgcgacctagatgaccttcgtacagttccgactccctcgacactcacacaactactctgttttctcgggagagatctctgcgcgggagccgggaggtccttgcttgcccccgtggacggtacacaaacgcatgggccttccacctttgggcccagaccctggtccaatctggagcccttgaacctcggtggataggcaggccaggggcccttttctgagatccgcccaggaataaggaaatgcttttcgataatctcggttttgtagatcccagacctgttgcttagacttgtgtgtgtggccccggacgttggtggtgcacgcctttaatcccagcacttgagaggcagaggcaggcgtatctttgtgagctcgaaaccagcctggtctacagagtgagttccaggacagtctccaaagccacagagaaaccctgtctcgaaaaacaaaacaaaacaaaaaagaatacgtgatgctccacaaaggacctagatttggttccttgcatctgctatatggccgtaattcattcctggggatccaacgccctctgtcggcctccacagacattgcacacgtggtgcacacccacacac

SEQ ID NO: 63 - DNA, 845 bp, Cricetulus griseus
gtgtgtgtgtgtagtgctggggtagccatggtcccagctccttttatatattatttttcaattttgagacacaatcacagtaagttgccctgactagccttgaactcactatgtagatcagtagatcttgagcttttgatcctttttttttttttttttaagttgggatttcaagacagggtttctctgtataacagccctggctgtcctggaactcacactgtagaccagactggcctctaacctggagatccacctgcctctgcctcccaaatgctgggatgaaagatgtgctccaccacccagtgagctttttatctttttgtgtcagcctcctgagtagctatgactaaggacctggaccgcaggcttagcttatctacttctgaaacagtgtcttggcatggggcccagacagcttcagtttcccagctgtttgggcttgtgtgtgtcacaacacctagtttgcatgctattttttatctgtaataacttctttaaaacatttggttttgttaattttgggggggagagttggtttgtttttctgagtcaaggactcaactgtgtagccctggctggcctggctccctatgtagacaaggctggcctcaaacgcacagaaatctgcctgcctctgcctccaagtgctgggattaaaggtctgtgtcataatacctggcttttaaattatttttaatggtgtgtgtgtgtgtgtgtgtgtgtgtgtgtgtgtgtgtgtaacagttagacactcatggagaccaaaagcatctgattttccagagctagagttgagctacctgacacggtcctgggaactgaacctgggtcctctgcaagag

SEQ ID NO: 64 - DNA, 388 bp, Cricetulus griseus
atttgcaggacgatggataaaattgaaaaaatattatattaagtgaagtaacttgggctaagaaagacaaccattctgagctggagagatggctcagaggctaagagccctctgcttgttcttccagaggtcctgagttcagttcccagcaatgacatggtgacttacaaccatctgtgtctgtacagagatctggtgccctcttctgccctgcagtgcagggaacatgcaggtggaatagtgtatacttaataagtaaataaaccttaagaaagaataaagactgtttgctttaaaaaagacaaacattccaaattctcttttatgtggcaatattagcttctaatttttatgtatgcatatgaatttcgactaggaaagtaggggg

SEQ ID NO: 65 - DNA, 375 bp, Cricetulus griseus
gggtgcattgtcctgggggggggggaatcaccatcatggccacatggtggcagcagagaactaattaatggctgctcccacttgagaatgggttggccaaggccatctaaacaggcttgtgaaggtccccaccccccacctcccaagtacccagaaatgtctcacaatctctggaggcaggaccacattcacaagaggccctgtctggatgctgtgtagagagtatttcagttcttctctgcatgactatctttagacatgttgactatggaacactggaagggtgccatggtttgtgttgtgtatttattcatttctgggccagttccacttagggaaaagagcaccttttatgttgccttgggcatagtattc

SEQ ID NO: 66 - DNA, 452 bp, Cricetulus griseus
agtctgaatacatactcacacacacacacacatgcgcacacttctgagtcaccatcataactagaggattgttctgtgttttttgctgcaggaagatagcaagggacccaaacctgatagtcaaggtgtatgtggaaggatggtgtcggagggtggttgtgtgtgtcaacttagaatttttggctctcctcttgcctttcagggccgggctgaagatccaaagccagtcttatgctgccccctggtgcctgcactgatttctgtcttgcacatccctgggcgccatacataggacatacatagccggacaagtcagcagatggcaggaacaaactgcacatgtagtatagtcgtggactctgagcggagctctttgcgtccaacgtgctaacttaaaagctggctggtttattatctgtaaaatggagcacacagggtaacttagctcatgc

SEQ ID NO: 67 - DNA, 1122 bp, Cricetulus griseus
tggcccaggcccaggcttggctagaatgtttcatgctgcctctccccaggctcagacaatgacacaaatagcatcagtcaggaccagcccctctggacaagagacagctcatgtgcaagagtgaagagcaggagcacctcagctgaaacgatccaacactgagggctacaagggacaagtcctcctttctctttagatgacttcctctggcatgggacattcccaagctgctgatgtcacaaaacagaaaaaggttagatccacagtgaagctgtgaccattagtaacttctacatgggactttatgaatttgcctattttttcattatcattatatcattattgttattagtttggcagagatcatttgaatttggctttccaagtactgtctggaaaaacgtctggccacacaccaggctgccccagctcagctgcctgggactgaaatgggcactgtggatacaggcacagtctcagatttcttgttgccggcttctctgaccttctgagtcacacgacctgccgagaccggtcctctcatctacccagaggccgctcaggaaatgtttggctcgcactcttggactcatgtgccaaaacagccgagatagcgatctggctccaaacaattccagctgtaagtaacacacttccgactgcaagaagccagctaccgggaacatggcccagtcctgcctgcagcccgcccccgtgtggtgccccaggtgatgcctgggaaacacgcctggaaagtatgtctgttgacagcaaacagaatgagctaggcgcagaacagcctgccacaacaggaactggccactcaccagatttctaagtggcattacattctgaaagaatcgctgtaaaaatcttttgaacttggctggcttagtggggtattcgactgatgatctgaggccttcagtgtaaacaaaggagagcctgtagaatagcactggccacagatggaaggccagcaattggagtaaagaagggtgttaggtttctcacgttcaagtcagtctcagagggtaataggccaaagggcctcaatccagaatgacttcccacacactctgctagggaatttggtgtgtgtgtggcttaggatactagtga

SEQ ID NO: 68 - DNA, 1344 bp, Cricetulus griseus
aattatttattatttatttgtggtacaggggctagaacacaaagcctcattcctcttgcatgcgaggcaagtgttccaccactaagtaagatatatccctagccctgcaaatcttagctttttattaaaccaatcagagtggcacatctttacagtgtacaaaaggattattccacaacacctggattcactctctagctcaagatcatcttaaactcatagcaatctccttgtctcagcctcccattgtgtgactactgtgggtgactcacaggccagctttataaacaatatgggctgctgagatggcttagtggctaagagcactggttgcttttacagaagacccaggtttggttcccaacacccacatggccactcacaaccatctgtaactccagttccaggggatctgatatccctgggtaccaggactgcacctggtacacataaatacacacatatataatacatacatacatacatacatacatacaagcagaacattcatgctataaaacaaaatacatttttaaaaaaccaaaggaggacaggagagggtggttaaaggccaaggcagtgttccaggactttccccagggtgacatgaatagatgtctgctcagtccagacagggccctgactgtagcccaaagaaacagttccacccaagtctagcccagagaaccaatgagctcactgggttacttactggggcacgcttaaaaggtgacttatgggagcatgggtgactcaaaggtagcactaaaaagtccactccagcaccgggtgatgactcataaaagtttccttggtgttttctgcccagcttgcaggcatctccaccttagagtctccactctaataattgttaacttcttatgtaaccttggggtggggccctgtaactctcattagctcccagagtcttatggattttcctccaggagggaaatgtttcaattaggaggaaattcttacatgacatgtgggcatccaaaatcaatggcaaagtgaaatataaggatgagagagacagagagagagacagagagagagacagagagagagagagagccttcccatacaaagggtttcttagccataggcctggaccagtcaaccagacatcagctggactatagattctagagtgtaatgggcatcagggcaagtggcgggtggcaaagaagaaatcttgtcccctttgctcttatttccacctcagaccttacatcttatgcttgatgtcatgagcctcaccccagaaatagtgactcagaccctaaaatgtcagggctggaagtgccctcaatgacagctagctctgtgctgtccgaggtg

SEQ ID NO: 69 - DNA, 1947 bp, Cricetulus griseus
taagacaactattaaaattggaaccttaatttctaccacaggtccttcccatggctcagctgcaggctttgttagtggccactcacaaaggcctgaggcagtgtcagcgaacgccttcaacctcgtggtctctaaaatcagcccgtagggctctgcgttcctcaaacgcacgcccgaaaaaaaatgaaggcctctcgcggtgactcagcatgctccgccatcagagccgttggtattcatcaacaaggtcagttgtccctaccaacatagccttcatcatagcgagaacatgggtgccgagtttccaacggagactcttaacaagcccaataccaccatgtgggtcgcgcaagcatgcatgcaacgccaagctaccgtaatctccatctcatgggccttaaaactcattgctaagcgtcctccagcttgaggcggctccacctgcaaaagactagccaacaaggcctaaaattaccaaaccaccaaggactcttacctgcccgcagtcgcctcacagctccgaaaggccgatatggcggctgagtcctgtatataaggtctccttgacgtcacgacgcacggccctccgctgcgtcctcttaagtcccgccccgtctctcagactcctcctcaagccccgcctctttcccgctctcaagctctcccgccggccccgccctcccgcgcctggagggcggagtcgccgcgagcacgtcgagcccggcctccgcctccctgaggagtcagggctgaggggagggggctcccgcaaggaaggcgctgggggaggggcggtgtgagggtgactgatttccttccggcacgtcgcccgctccggaggaggggggcggggggtttcacttccgggacggtgttccggcccattccggcccatttccacccccggaggtaggtgctgttccttgactgggctcgggccccgctcccccgccccattagcctataccggacgctccttggccgcgtctcggctgcagccagctcctggggcgagcagccgctccgccccacctcccctcccagcgctcggcgaccttccccgcctctttggctcggggctgcagagcttgcgggcccgcccccttgcgagcccctccctcaggaccccgcggcggggacacacccagacacacccccaccgcggctgcctccgccgccctgaaccgggtcccggggcgccgcccccttccccctcccgcgcctcacctgtgcgcctgctctctttgcccgggttctgtgccatctcccttcccgcttcgcaaggtccccttctcccttgctccctgacttaacacggaagctgctccagtacctgactggccaaccccaagcgttcccgattctcccttttacagctctcgcccctccagtgccgacgctagagaaagactttacttcaggggcttcctcctcactggggaattgctggaaagagcaaacaaaaaggcgctggtgtaggctccaaaataaacatctgaattcgtgatggcatcgacagaggctcttaatggattaaaagtttcaaaacaaacaataacgctttggaaaaaggtcagattggttaaagttgctacttgaaagcagtgtttgcacttccctgtttactcaatgaatgttgtaaatgcgggctgatgatgtgagcttctcaggaccttcctgatgtgtagtctcataaatttagagatgaatgggatgcacagtgagccacagtcgtcgtgatacatccgagctaccctcaactgagactgtttgatgcagttgcagtcttttagtgtgaaatttcaggctttattgtcagctttggggaaccaactcccttgaattaaggtagcattgccactttggaattaccaacctcgagtttgttgggaagagaaagacttagtagctagagaaagaaagatttacttaatttttgcctagcct

SEQ ID NO: 70 - DNA, 2363 bp, Cricetulus griseus
  modified_base (258)..(472): a, c, t, g, unknown or other
tcctagacctttggcccaacccttaccctcctggggactggggacctccaacccacaagcaactcccatttcccctaaatccccttgcagattgactcaagtctccatgaagcccatatttctcccactgcgctcccggtaacggtgctggccaggtctacaggtccggcccggacccggttgcgtttagtcccctagggagggacgcagggaaggcggagccacattgcaagaggcgggggcagagctggcctccannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccaccccccccccccccgccgtaggggaagcccgacagcccagaagcttcttcaaaagcttggtggcccgagtcctacccagcgcccttttcccctgaacttcgagtccgctgatcgtgggaccccaggatcagaagtcaagggcttctctgagttgcccaatgtgtgcatatggcggtggcggtgggggcgggaaagggtgcgaggaggtggagccaggtcagcacgaggagggttaacccccggccggcgggcggctctccctcccacggggaaatctcagccttttctgcagcagaacgagccgtgcgagcaagggggaaattccccctcctccggggcaccatgaggtctacctctgggtctcgctcaccctccagccacaccctgaacgggaacccttttagcaggggtggaacaggctggggggggggggctggagcatcaacttctagggaggggcagccacgtcccctacacccagaagacccccgtttcctctctgccccgagacttttccatgtcccgggaggtgtaaggccatgggtcggggaaagtggtgtgacgcttcggggaggggagtgtggaggcctgtacccacccgagtccttccaaagttggaaactgccaaaacccagtgccatcagccactaacaccagcagaaaccatttccaggggaccaaccgccccgcccaggggaggatcccgagaggccccgaaaatccaccccccacacttaaccaaaggccccccaccttgctcgcccgcagacatcccctcaccttggcagcccctctgctatcttctccctttctcagcgggcactgccaggccccatgggtctgtggctctgtccagcgggttctgggctggccctaaaaactcagtatgtacacgcacacatcacacacgcatacacacacgcgcacacacatacacacaaacacgcactcagctccaggccttcctcttcactgtaaaccctggctgccccggcctgctgtttccggcttctctgtctgcccaggtccctccctggagctctgacgacacagagaagagaaagtgggctggaggaggaagaggaagaaagtggggtgaacatgaatgcctgctgtgcccggggggaagcacgatgaagcgggcagggctggagctccactgagtcactggaaggaggctgacctgcgcctctagccccctaacaatgacccattttttcctctgcaaagtcacctactatctgggagaggcatgccaccctcaaccgctcaaactttccccgggtcggaggtcggggccccgtgggactgtgcgctggggcatctgaccccactgggctgtgcgctccctccagtggaggaccctgagaagtagcccggagcctgcgggccccaagagctaggttcacctccttgccaggcgggaacgccttagaggtcccgtgacgctgtaaggaggaagtgggggaggggaacccttagctcagaattctggaagactcccagaggcgctggaagggagagggatccgcacagggtaccacgcctcagaccctagtgatagccactgaggactgccaggagagtcctagggggtcaagcttgaatgtagatatgcaaaatgcctgtctgtttctggggtaaacacggagacacaccccaatacaaacatgtataatttccctagccacaagactgcatgcacacgttcacacatgtaacggcaggctggcacacatgtcagcactcggagccacttagtgtctcacacacacaccttgctcggccacacacacatgcacacgcacacacacccacacgttcacacatgtcagcactcaagtcacttagaatcacacacacacacacacacacacacacacacga

SEQ ID NO: 71 - DNA, 1790 bp, Cricetulus griseus
attcactttcaattttttgcttttataaagcattgaggtatgttctttatgcattatagattcaagtaagaaataatgacctcttcttcactgctgaaccatctctccattccccgtcctagtttttctctgtgtcacaacagacttaactctttggatgacaggtcaggtagggtcccaggctccagtcaacattagttctctacgtcctcaatgctcgacccagggcgccccctggcggtcagaagctgtcatcgcccgtggctgtgccggtcccttcagcggccccagggcggccggtagggagtgcgcaggcgcggtccgacgtggcccgggcctggctgggaccggagtgtgtcgggcgtgtacggcgctctctcagtgggctcccgggctcggagcggaccgctggcctccggtgcgctttctggaaggtcagagtgggcaaaagctcagctcggcgcgggtgtggtgacgcgtgggcgggcccggacacagagggcgtcactcggggctttgtgagccagactaacccggggtacgccgcgggctgtggggggctgcggcccgcgccacgccggcatcttgcagggagctcttctgccgcagaagtttccagaagtcctgcagagggtattccagctagtattgaaggagccttcgccctccctgcaccctccagagtgctggccccgggagtggcgctcaggtcttagatcctcccaccccaggacgccgcgcatcccagtgcgggtcttcccagccctggtactcgcttgttccctgtgagtccttccatacctggtccctgcggattcctaatctgagtcctcccatccctgatccctccctattcatccctgtgtgcgtcctccacggttctctttccagaccccactggctttctcgctccccagggcctacagcttgcccatgggtgggcagccagctccaggagccacagcactgggaatgaatggcgtgggggtttgctcattggaatagactggttactggcattcagcgtctctgaggacccctgcggcctaacttcatatctggaaaaagttttctctgggaatgcgcacggttttgtgacgcggcattattgcgttagtgtatggctggctccgttactcccatggctgtgttatttcttgatggtcctcgaggtttgccttgttgtattatacacttaacgattttgccttctaatttttccttttttttttaaatctcgataaaatgaccctcggatatctaagaagttagcaactatcttaaaatttacagaagcaattaggcgaataataagtcagtagaagagtactcctagttcctagggaactgctcttggtccgtgctcttttctgaccgtcttaatcgatggtcgtggaggacttgtttgaaacaggaaacctctgtactaatgtggaatgtggtttgctgcccatgcgtggtgctttctgaaacatacagttctctagcaagccagaattggctagaatggtacaggagtcgaatagaaacccacccggagttttagttccccatgttccctaccaggataggagtaagtgatgtagtttttggatcttagttcagtctgctgactgtagctaatcatggaatgttgtacacatcagtgggactgaatttcagctgtctcattttttaaatatcatatatctggggttttgagtaagctaattagctttatttcatcattccatatttcattttttaaagaatcagaataatggggctggtgagatggctcaaccctggaa

SEQ ID NO: 72 - DNA, 502 bp, Cricetulus griseus
tagcttccagccgtgtttctcgatttagtaggcagggtagaggagtggtttctccactttctcttaaacgcagcatttcccaccatgtagatccctctaacaataggagatttttaggacggaaggggccccataagttgtaaattcagcatcctctttggtctcccacaagagggcagcgaacaaaccgcctctgagtgaatgctggtcccaggactgtgagatgtcgccctctagtggacccagagggctcagacagatggaggagggagagcctaagagatgggatgcaagaaggcccagaggcatccatccccatgtgaccagccccattccctgggccatcctgaatctctccggcaatcttttcaccatacatcattccctggagctcattcatctggagtggtgaccaagcaccctcatccacccacccggggccaagctcgggccaggctgggaattcctgaacttacagattcttgcttgttcccacccatgt

SEQ ID NO: 73 - DNA, 631 bp, Cricetulus griseus
caatggctggtggtgatgcaccccatggcgcacacaggcctgagggtgaccggggtagggatcttgtctctgcaatcatgagaaggaaaaacctcctgaaaacgtccacgggccacttgtgcaccacctgctgggaagccaccagggaacacgtgcgttcccagcgaggctgccgcacgggatgctttggggtcccctggccgagccagtttcgctctcctctgtggccactgcattcgtgctgactaatggttaagagtcagcaatgagggctctaccgaaacgcatacggaaaaaaaagttttgatgtgaagaggaaaagcaaaacacaacaaaaaatgggaaactttgtcacttggatccttaggtactaagtcgccagggaaggtagggtagggggccctcgaggagaggacttgacctatgaggaagagaaaggaaaagggaacggaaggcacagctagcacatcctcttaaccgccccagagggcacggcggcctggggctgtctttggagaggtccatccctccttgtcaatcagctccacctggcattgcgattggctggtggcttccttgctccccgcgccccacctcctagttgcattgggtttacttccccaaagtacac

SEQ ID NO: 74 - DNA, 435 bp, Cricetulus griseus
gcaccagcaccctccatcttgctagtgaaaagaaggagagtcccagcacttgggaggcagaagcaagaggctcaaagccagccttgtctgcacaaggagttggaagccagcctgagctatgccctgttatcaccccctccccccaaaagaggagagcccagcattggtgggtgagtcaagattaggccactgatctggacatcatgtggtggtttgtgggaggccatggacgaggtgtcagcagagcttgagatcccagacctgaaacggaggcagagctgtgtcccgcctcagggagtacaaggctggctcccacctggcctgcagcatgctgaccaagggcttccatattcctctctcttttctcccatcctcatctccagtcaggcaagcttccctttttcattatcgaacaacactatctgccatgtgctc

SEQ ID NO: 75 - DNA, 1254 bp, Cricetulus griseus
  modified_base (542)..(923): a, c, t, g, unknown or other
gctacagaacatgaccgcagcacagagtggtcaaaacgcgccagttaaacgaagctgacttcatgtttcacaaatgcctgagggatgtagaaacgaaaagtttccagtaaaacggaacgttctggattcctgtaagaaattaacgtttggaagcaaccctatagtctttaaggaaaaaaaaagaaaagaaagaaatcacgacagcatcgtcgtgcgaacaaaccatcaaaacaaaactgaaaaacgtcccgagaacaagtgatttcgaagcccgagagggcagtaggagagcccaggcgcagagccatttcagctctgccctaagtcccgacaccgcgccgccccttcttgaaaccaggagcttggaaagaacattcccgaacaggtcctcgaggatccactacggagcacggagtcgaaacggacgtttcgaagacggggggcaccgtcgtctcccgggcagctctcgagcgcgcacccacgcgcaccccggggcggcggcctttgtggtggggctgtgactaacgaggcccccgttcgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctctgcggggaggacacgctctggcccccgctcctggcccggacagtcccctccatttggtcttggctttcttctatcctttatgcgccgaaatacgcattctagggggaactcagattactgcaactcttactgtgcttgcgctggtaaggctgttggaaataaagtgaggaccaacaggcaaactggtcccacctggtgggggaggagatgctgttagttagagacttagctaaggtacttgtacagctaaacgtcattcgttcaccactcgtgcattcatttttttttaaactcatctactttgcaaaattttcattaagaacactt

SEQ ID NO: 76 - DNA, 919 bp, Cricetulus griseus
aaattaaaaaaaagttggaaaaaaaaagtaaggaaaggagtaacacgatcttatcatctaaacatttaaatagccacctgtccaacaaaaaataaacactacgtgaacttctaatttttcagcaaccataaccatgtaagcatggcagggagggtgcgcctgagcgggtagagagcgcctccagtggagagagagcctaggcgcgcagaccctaggccccctgccgctctttttagctaccaaaacacattgagagtcccgccctttcgcagccccgcccacccggtggctgtcactcaacagggactctggaccttgtagttaccagcccctcctttcttttcttccctttcggtgatgaaacccatgaaaaactaccaagcccagaaggcaccgcacgctcccgaatgggaggagcggcgggcgagagggcgggacatgaaggcctcccggaaaggattcaaatttccaggactgaaatcgcgggaggagccggctccgcggctttaggtaggagaggctgctgcgtccttcccaggacccgggcctgcgttctcgatggcggttggttgttaggtttttcggtcagcaccctccctcacagcacacagggatgcagttacgctcggccggaccggtttgggctcccctagaagtagtctggccgggcggtgtcctgactccgctcccctgctcttagtgacatttgacagaaggagtgggtggagggactggagatggcccatagggtgctgagcactgttcgctcttcccgatgacccaggtttactccccagcacccacagaggggggctcgccactgtctatttctccagttcggggatctgacgcccacgtttggcttccgggggcactgcacataacgtagtgctcagactctcgtgcaggcaaaaacacc

SEQ ID NO: 77 - DNA, 329 bp, Cricetulus griseus
aagttggtgatcatcttgggacctgcttcctaagcgcgtccgtctgcttctggccactagggggcagtgctgcacggagacccactttcagccaccctgaaaagggcccctttctcttccacacctgtgaatccagctgggcaggtctggaaggcctctgtcccaggcgactgccggagaagctcctgagtcaccatggatgctggctttggacccacggctgcccccggggcttggcaagttgcactggaaaaactgggtcaggcatgtggcctagtttgctggttcgcagcacaggccagggatgaacccgggagtgcgaagggcgt

SEQ ID NO: 78 - DNA, 485 bp, Cricetulus griseus
tctctctctctctctctctcacacacacacacacacacacaccccaaagtaggtttttagtaagctttaacatgatataaagagatacaaccactaaaagttttaagaacaacaccatctgggacattagaagcaacaaaggctttatcagctcctagcaaacgtctgaactacaacactgacatctagtgttcatgctgatgaacctgcacaaaggcagcaatgagccacactgaggcagagaagtcttcccatgatgcaagccactgcagctcaccacctactggaggaatacaacacaggcaccaacctgtaaaaagtttacagcattattttatacacatcctaccaatgaaaaaaagtgatgttcttgttttggtttgtttttccagcagttgaaggcctttattaggggtaaggaaactcacatgcagcagacacaaagaaagacagggaacacaagaagccaagaaatccctattttt

SEQ ID NO: 79 - DNA, 1272 bp, Cricetulus griseus
  modified_base (245)..(261): a, c, t, g, unknown or other
tccccccaccccaccctccagcatagcatagtccccttaatggtgagtcataccccaccacagatgactcagttcctgatcttgtcctgccttctcaaataagagctttttctactcaatgaattttcactgttctttgaagttctggcttcaccactaacttgaaaaaaaggtgtctctgaatggcctgggacttaagaacaaaaaaaaaaaaaaaaaaaaaaaaaaaaaaaaaaaaaaaaaannnnnnnnnnnnnnnnnaaagaaagaaaggaaaagctaggaattgtgattaggggagatgggttagtttcttatttcaaaaatatgcactgctgtgcaaacagctgctttttatgttttaagtcctgaatggaatgagtgaaattcagaagctagcttaatgaactgtggatcttcactgagcactgctaaaatcccttctgtgctgcagcagcgtgtttgtgcagctctaaccaggaggtaaactcaaaatctgcctgcaagtgatagtgtctgctcagggtatggggcttggcaaatcagctggagtcatccccggagtgccacaggtgtctgctcctctaatgccaacacctagtagttgtactctccctctcctgcttttggaacataactaaaaaggcggccgtagttcttcaccttcttctcttgtctttcatctcttgagcagttctctcagtctcccccaggtcccctaaaagactaaatacaataacaagaaaatagttttctagtgtgtcccccctcccctgcccccttcttgccgcttaaccacacacttgctccatcctggtctacgcccccacctagtggtttaaattcgccagttgtccttcccttcagtttccaatagcttttaactgagcctggtggttaataaaataatcaggactcctgaggtgggtggatggagttgaaagagggggaacacctacaaatgtggaagaaaatgcttaatctgctgaagtttctggccttacgaaaaggaacagagaaaaaataatctttttttttctttcatctactgcaatccagctaagaccccttagtaaaaggagcagcctcttgaagcttggatgtattctcttccccacacttgctgtcaaatgaaagtatagtgatttagttgactatcatacactgtctttgcctctcctaggatataactgctgaaaaaactgggtctgaatttgccctgtttgtctccatattcctagtgtggaggagaatacccagcatcgattatga

SEQ ID NO: 80 - DNA, 1119 bp, Cricetulus griseus
  modified_base (96)..(852): a, c, t, g, unknown or other
atctccatccttccactcttccgttctccttgggaactggggtctccacgcccgtctctcccttcctttctgggccctacactctttatacctccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntccgtccccagatgctgccctttgtggtcgctgccctctggagggtgggatggggtgtgtgtgttgcggggagggggtggtccggcccccttcgaaatccagagcgaggcaataacgtttgtacgtgctaaggaatggtgcaaggcggtccgagtcatttggaagcttagccttttttttgtcgcaagaccggggagacaggatgagaactgtcctggagtgctccttttgcgttttcacaggagctactggctcgtgactcaggaa

SEQ ID NO: 81 - DNA, 528 bp, Cricetulus griseus
actggaaactccttagactaacttgatttcaagacattcctttgaaaaacacttgaggtaagaatttcatcaagaagtcggccccacactgaaacaacaaatccaaacattagtgtttgtatgtctttaactatttctgtaaattcgtcatattccactctcagggattggagctaggggttgtgcacgccaggagagctcatgggtgacggggacatctagaggctatagtaggaacttcaggaagctttgagtgctcggcattactcaagcaagactgcatgcagtgtgggaccctgccacaccaaatccagtagattctgtgtgacaagttccacagactgaggtgataacttcagaaggaggaggtctcctggaaatggttgtagaactctcagctggggaaaaatattgggctgtgattctcatggcaatttctgttcttctattcctttttttttttttttttcacaacgaaatgttccaaaaagcctatgctggcattttaaacaaaagaaatagataaga

SEQ ID NO: 82 - DNA, 920 bp, Cricetulus griseus
ctattatgggatttcacgtgctttcatttaagtctgtgtttgctagaacaattctccagaaacttcctgagaatgtacataggacatacaatgttttgaatcacataaatgaccctttaggactggtgcggtgtctctgtgtgtcaaagtgtgtgctcccatgcctgagttaggttcctgtgacccacatggtaggaaaggagcactgactctgcaagtcctttgacctccacgtgcagaccatggcatctgcccctctgtaaacaaataggtgtaatgaaaatgtctatttacattttgtcagtagtttggctgggggtagacatttagatgtgatcatgactcattgactacttcagaggatgaggcgggaggggccgacttcaaggagtggctgagtaactgtgagctccaagcttgcctgtgtaacttagcgagcaaaatcaggagtgaagaagtgggtaacaggggaagagccttctaggcaactcccaaggctcagccccccgccccagggctccatcaggagaagatcctacttttggaatttttttttaaacttacaaatcattgacttcctgtcctctagctttcagttttactgtgtttaaaaaaggacaccattctgaatctgaatcctttgtcattatctttgcatatgtctttcttttaagaaagactagtcacttctgatagacctgacaagcttgtcttgcatttttgatgggcctgtcccatctggggatttgaggacttcagtccggtaaactctattccctttctgtcatttccgccttttctttcccagaaagccccagggtttgagaacatgccttctaccatactttaaaaaagggttttgtttatcagggtttttcctttccccacccattttctaggagatttcttcacctttggtt

SEQ ID NO: 83 - DNA, 717 bp, Cricetulus griseus
accttcccccacacccccataactgaggcatgcgggagagtgggctctgatatcacaagagtgagagagctgccccgaccctcaccagctgcagcgctcaggatcgtggcagctcagcagagctgacttatcgatgggggtatggatgaatcagccccaagaatgtgagtgtgggagatctggccccatccctcatctgccatatggtgcccgcggggcgggggtgtcactccctcttgcctgtggcgggagagatggccctgaggtcacaagaatggaagagcttgtccctgcccctcaccagctgtagcacttgggagagtgttcttggcacctcacctaggccacacagtagagctggccctgatgatccaggtgtgggagagtggaccctgcgggcaggaaagcaggcgaactggccctgccccttgatcatccctgcaaagggtaaactgactgggaagtgctggagagctcactctagtggtgagaatgggggagagcgggcaggctgacaacccagcaactacccaggcccagaacccaggttggctcaccccaacatccacccatccatgaactgcaggagcgtgtgaaggggctggtcctgtaaacccaaagctgcgggatctccacaacacagggcaacaacaggatatccaagaggaggcccagtgagggcccagcatcgatagtgtagcagaaaccagaggcctt

SEQ ID NO: 84 - DNA, 366 bp, Cricetulus griseus
gatgtgcagtggcagcaaagggtttaatggcttcttcaagcaaaaggagcctgagctcccaaatagatcagcaatctcctaggatcaaaggaaaagctgctagtcaacagagactgcacagatgcaagctgcaacagtgccacctcgtggcagcatagagtaaaagcattcatgtgtccgaccctgccccttcgctaagagtctaggcctggccatttagttcttgattctcctgcctcagtttgccccatcttggcttgtcgtggacttctgataaccagccagatggttctacaaaagatacttgatggcatcacagctgattactgaagaaactatttaatctgaaggggtgacaataactcg

SEQ ID NO: 85 - DNA, 482 bp, Cricetulus griseus
acctccccgggtcatcaggcctgaatcttctcagccaaacctgttttctgctgtgaggtttgggggtgggatggggagaccgactgtggaagtccctgaaataaagtgatgcaattagccctcacatgcttttgctgtctctggagcgccgcccctcccccttccccttgggactgccattgtgggctcctcccccactctggcgggaccagatggtccccctgccctttgtccactaggccagatggagggaaagggagattggattcccctccccacccacacacacccaggtgccaagagattcggcttagggggggaggtggaggtcagtgggaccagcatgagcggattacggaaagggggagcgtatctgctttgagatgggggaggggcaggctcggtacacccccaccacacacacacacactcctatccctttcctagtaaaggatttaatggcgcttcgtcccttataaccc

SEQ ID NO: 86 - DNA, 729 bp, Cricetulus griseus
aactgtcagctctgtctagttccagaacattcctcagaagaagccctggcttcatgagccttcaccctgccgggactgcaggcagcctcctgcacacactgtccacacccgcttcttgggttcacctggacatttcctgtcacagaggttacattgtgtggctttctgtgtctggtttgctctctgtcgggttgctgtagggcaatcctcttcatggttgagtaatactccatgtgtctgggctctggtgagtcactctggtgaggccagactgtgcttgcttcctgttagaccatcatttcaactactcagctgttggatggcagataactttgtctcagataactctggcttacatgtgaaagagccctgccaggggactctgtttaattaatgagttagtgaggatatcttttaaaacttgcacaattagacacacatattcaatgctgagggactgtatccctccacccctgtgttccagtcctcaccccagaacctccaggcctgaccttattaggaattaggatgctggagggtgtaattaattctgatgaggtcatgttggggcagagtgggcactgtgccaatagagtgtccttttgagaaaaggaactttgacacagacctaggactcatgaggatgatgacagaggctgagactgaactacaggccaaggacagacagggattgccagagtcaggaaggcctcccagagcatgggagga

SEQ ID NO: 87 - DNA, 334 bp, Cricetulus griseus
gtgtttcatctgcatgtatgtctgcaccccagaagaggtaatctgatcccattatagatgactgtgagccatcatgtgggtgctgggaattgaactcaagacctctgtgagagtagccagtgcttataaccacagagccatatctccagccccctcaaccgctttcttatagaaccatggactgcctgcccagaagtgagtaaaacaacagtgggttgtagtctcctgtgtcaatcaagaaattccttcagagacctgctggaaggctaatctgatggggacaattcctcatttgtgtctccttctcagatgactccagttggtgttgacaaaa

SEQ ID NO: 88 - DNA, 2525 bp, Cricetulus griseus
ctgccgctggaggctaaaagagggggaaacccaagcaaagggcactctgaacggtcactttaaaagaaacggcttggctttgccgtctcggttcagggatctgagcacgtgggccggcagcccgtagtggcccggggctggggatccgggatcggggctggggagcacgcagctcctaccttcagggctggaccatggccaccctcctccggacgcgccttccgctgctcgccgcctcctgcggcctctctcggggcttccgggacgccacgcgccctctgggcactcccgccggtggactccgacccgcaagctgccggaccacgagactccaggtccgccagaactcaaaagaagcactgaggggcagctaaagacaccgacctcgcgaccacacaacactagctcgtcccagcatggtctggcttttgacactgcacctggaggatgcaacagccagggcttcctggaatctgaaaccacagtccatccacgctctggatgacatcaggggaggatggtcccatttcactaaggggtaaactcggtaaagtctgggacaattaaaaacggagcccacagctatattgataccagcacttgaaagaatagttattcagtactaatatcaacagttaacatggcataatgatataatgcaatgtttttatatatagtatttagtcttaacagctgtattttagacgtaggtattaccacatccatttcacaggtagggaaactgagctacttctagccaagcaacttgcacaagttggagcagccagtcaatactgtaaacaggactcaaatgcagacaggacaggctgcaccgcctccttcagttagggaagggctgtgtagcagtctcagctgccctgttaaataacagcctaggtcgggaagggcaagccacttgaaaaacatgacatgtgcggcacaatatcatggagtttcttagcatgtgaaaatacctgtaaaggcatgcaatttcttaatgttatcactcatattcggcacactgccacatatgtataaatcaagacccttgctctgcaaatgatacccattttctctggtagcctgcaagccaccagtagccaaggaccagactcaagtccccatatactgaagctttgtggccctggctatcctgagcaccctgccaagagcttcccttagctcgaccgcaaaagacccagccctctcagacacacattgaatgtgtgccccctccccttccaaatcaccaccccggagcgatgctctccgacccgccagcgaacttaatgagcaacccaggcccctggcggtcagcttcacgctgacacagagcttacgtgaacaagcacgatgaaaacaccagctctcctcaccccgcggcgtcccacaccttccgcgtggatgcaaggcctgacggaagcaaactgaggctaaagactggctggttcctagtggctctaggaagccacgccctgctcttagtaggcgggtgaactgacagtgtcagaatgacaaagcaaaaatccagtcctcgtgcagccctggcaacctgtacccatcggctcggcaaagtaagagctcctaattggacagcgcctattcctgatgctcagaaccgccccacccccacccaaggctagggctgcaattcaaagaatgttgggaaccagacccagtagtagttgagtgtgcaatgagtaaaccatcccctgcctgccctgggcgctgctctctacgaaatacgtcgtcatatgaggaactttagaaatactgactcctggcacccacacagatgttctgatgaccttggtactggctgtgaccctggcacctaggttttaaaaactccacaagtgattttagttgacaataaagttagggacgcactgccctaaggcaatggattagtcacatttagtaattccctaaaggaagttactgggatttttaaatcatatctgactaaccctgaaaagaagtgactaattctccttacattacttcctccttgtcaggcttggggttgtcttgttcactttaagggaaatagtatatttggcacttagcacagcatctgccaaaggaggcatgtcctctcatgcttctctacccttaggtcggtgagaatgcttggtggatagatgaatgaatgaatgaatgaagtgtttggcccactactttggttcttgccagggtgaaagaaaacaaggcgaaagctcctaacagcttaaggccacacctcctggtaactgggcggcctcctgcgttccttctaggcttttccaaggtgaaagtaaaaaacttgcacaatgtacacacactgcacaggctaacccttctcccacacttcccccactccacacacttctcatatgtatgcagtgcttgtaccatcacctcacctggactgaggtgttaggactgtaaccaagtgcttagtagctctttgtggccccaaatacctactcttaattacacatctaagtatcagaactttggaaaagcaaacccttgtttgattt

SEQ ID NO: 89 - DNA, 513 bp, Cricetulus griseus
agccatggagtcttgggagaatggagttctgaggggaaaagcagggatattttgaaccaacatttaacgtttaaagacaacccagaatataatggtatttagaaattctcaaaaccccaaattttcagagaagggagctgatgaccctgagtaggtacttcagtcaagagattaaacaagatgtgtcatgtaactaggcccattaactttgctgcacttctcctcgtcctttgctgccctctagaggccaaccaaagtttcaagctgggttcagaagtcttccttcgccttcagcagtgtttgttactctaggtatctaaagaaaagcagcgtccagctattgttttgttttgtttttatcttgttttgacacagggttttgtttatgtcactgaggctagatcctatatcataatttagtctaggctggaggtattacatgaccatcttggctctgtctctggggcattgggtacacactcaggactcaagccctcactcccctccttgttt

SEQ ID NO: 90 - DNA, 663 bp, Cricetulus griseus
tttgtctccctagacactctcaactttcatcccatctgtacatgaaatctgggatccacaaatgatagaaaacatgtgattttttttctgagactggcataattcacccaatatggttatctccaattgcattcattttattgcgcatgacaaaactgcattcctctttatggctgaaaaaatccatcatatgcacaagccacattttctgcattcttctgctgatgggctgattccatgacttagctagggtgaatacttctgtgatgaatactgataattatttctgggaaatgctgagtggagtcttttgggtaaatatctaacagtggtataccaggctatatggtagagctcttttgtaagcaatcttcatggtggtttccatagtgattgactagtttatatttgcactctctgagtaaaagggctctcttttgcctactcccttaccaatgtttattatttcttctgactgtcattctgaatagtaggagatggcattccaatgtagcatgggttttcatttctctgatggatagtgaggttccacattttttcacttttttgcccatttgtatttcatcctttgtgaattatctctttatttcatttgcttattgactgatgtggttatttgacttcttcattcaaatggtgagg

SEQ ID NO: 91 - DNA, 328 bp, Cricetulus griseus
ttctaacatatatgccctgctcttgatcttaatctctaataataaccttccataaactttggagtaaaatggaaccaatggatgataaggattcatgaaaattagaagcatgcttccatccagcagagggcacaagtaggttgggatcagagttcagaagcaaatcccttactctgtcccaaatcctgccagcattaccttccaaggatcattttcaaaaaagggggcagttatgactgtatcatatccgtagattagtcatcaaatgtcattctccttttaaatgaacaaaaaaaacccttcagtttccttaagagtaaatctacat

SEQ ID NO: 92 - DNA, 475 bp, Cricetulus griseus
cacactgggtccttggaattggagtcacaggaggtgtgagtttccatgtggatgctgggatctaaaccctggtcctctgcaagagctgtaagtgctcttaacctctgagtcatatctcaagttctgaagagctatttttattatccaccacaaaataccactcctgagcaatgtgatttacctacctcttagtcataggatcattcacaggctatgggtgggaatgcagaattctggttatcctgatggctcccaacccaggacagtcatcacttgttatagagccaggatgggccttttgtgggcatagggctgcaggactcaggactcacagtcaaatatacaaacatgtgaaggaatctctccatgtcacataaagacttaatcatccttcatgagtcttcctaaatgtcatttgtttagtatggatctccttttaagtccgggacaggactgatccaggcagtatgtgttc

SEQ ID NO: 93 - DNA, 1160 bp, Cricetulus griseus
  modified_base (243)..(387): a, c, t, g, unknown or other
gctctcagaactccccgagaaacaaccagaagcggaggacgggagacgagaaggaatatccatccacctagctaaggtccactggaagctccagtctgggctgaggccgcctgcggcccaaggtcgcgcggccagaccctgcccagctccagggcggagtccccctcggtttccccggtgagggcaaggccctctgagaggcactgacctggcttgagtggctggccccccgcctctgcctcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgcctcgaaacccccgcccctcccgggccccgcccccaggcccttccatagcgacgttggctcctcccaaaccccctccgctcggcttgctcctttgcagccccgggtgttagcgccggcccctcgcagtgcctgcctcatcaaaacgccccgggaatcctcctctgcctccctttcggtcttagccacacgatgaaaaatgccagtttcccacgctcggtgcgccgcttacctgatggcgaccagatccaaagcttagattcctagtccaagcctcacttcctgtggcaccacagcaattttggggctccagcctagctcccgcccaatccgtccctggagtactttgtaagtggggggggggtctccatgttccttttggggctcgaggacggatgcttccggcggccccccgggtcctctccagggccggaacgataatgccagagccgtcgttttccgagccggcggagttaaggaggaagcatacgtcagagcgctctcgccgctggggaaaggaagaggaggggccctgcgcgcggtatcctgggaacgcgtggtattctgggtaccccgggcccgccattccctcgcttcacgccttcgccgtgtaacgtttgtgccagggccgaaagggagccggtgaggagaggcggagaaccggtaagaggaggcctaggcgtccccggagggttaggggcaggaagggagcctgcggaggagctcagcggggcactaatggcgggcgggaagcctgggccgatccaaacac

SEQ ID NO: 94 - DNA, 805 bp, Cricetulus griseus
gtacccaactgaccccttcaaggcaaattcagacctttccactaagtgcaaaccagcctttgtccagtgcaaagcttatcaaactttaaaccatccagcctcacctctcccaccctcaacgacttaaaaattgtcagcctccttcaagaccaagcaaagcccagccctgctccggtgcccgccagcctccactgtgtcagagacacttcctccgggggatcctttgtgcctctcaccttccacctccccttgttggccccttcaccttgttctcatgcttttctctggatgcatttacttttgtgcatattccctcaccagcagttacctcactgaaggtgagtagtaccctcatgtgtgtgattgttgttaatttctttacctagaatatagggttcattggggcaggggtggtgtcttcttttgctggccccttttatcctaactgtctcctctggtgccgcgtacctagtaggcaccgaatataggtgttgatgaatggatactaatgcgtatgtggaagtttcatacccagttacagtacttggcactaacactgccatctagtggcaacacattattatgcagtggtcctagtctcagacactgcaaactctgtggcctcactccaggcccagctggccatcactctgtgtacgatagacacctgccaagcacttgccctggctaggttatcctatcaggtttttcatgccactctctgtttgaaacacgagtcagttacctctggaggtcactaataactaaggaggctgtatttaggctcttttgcggttttacgctg

SEQ ID NO: 95 - DNA, 671 bp, Cricetulus griseus
  modified_base (313)..(422): a, c, t, g, unknown or other
tgctatctgccaccaaaaaaggcagcgctgtaataatcctttgaactcattagcattgaaaaggaattcctacagcagggttattatctttgcacataatatagcctttaaagcctcccgttactgcttcttgtaagaacccagtaattgaaatcgagacgctggcgttcctgagtcttagtggtgggactgggcatgcccagtaagtagctccgccgtgcttgcaaatagcaagcttgcagggttcagcgcctcggggactggcggctctggagacaggtctgacttctttagacgtaagcatgcgggcacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagacgccgggtcatttttgccagcgctgctgtgcggttctcacgccccgggctgcagacagctaggaggctttctctagtctcaaccaggctgcgggtgctccctcccttcctccctcccgctcgctcctctttttttttttttttttttttttccccgggggggtttttggctgcatttgcatgaaatcccaatggtgtagaccagtggcgatggatctaggagtttaccaactgagacatttttcga

SEQ ID NO: 96 - DNA, 417 bp, Cricetulus griseus
aggcagcaggcagccaggccaggaagttaagcaatcacatttccaaccacgcacagaaagcagagaaggcaaactggaagtaagccaaggctataaacctaaaggccacttccgatgatgcaattcctccagccaggtcacacctcataaaggttccagaaactcccccaaacaacaccaccagcatttcagcaggcgtcttgtaggtgatggactactcgaaggaagctgttttccaggaaacaacatgctgatgcacacatgaactagagggacctggactaagtcaggccagacaaaccccagcacggagaaggcggagtgggcacgggagtgatgctgggtgagtcagccacactccagggcagacctaatctcaggcgtagttggccagcacaaagcagactccatgtttgg

SEQ ID NO: 97 - DNA, 641 bp, Cricetulus griseus
ggcagtaaggacacagtccatactgagagccagtgaattgcccatcctcaacaaggggaaaagttggtgaacaacgcccccttgtgtacttgtggtgaaacaacaatctagagggggtggcagtagtacatgaagtgcatcccaaaaggttcagtgggcagcacaggttcagtttgtgcccctgatcctgaactggtgcctgagcatagaaggtatttcctctggtcctggaggcaaaagatcttgttcccgcttcctgtctcagggctctgactgctcttctttttgatgcatcgccaaatggttgattatatacgcatgcatgcacgcaagcaggagtgtatacatttatcctgtggcaggtctccttgtcccacagaaatgagacacagcagcagtgtaagatgatgggaacagggaagaatgctgtgggggaaaccgggccactcccacattccagttttctaccctgagacgaacagccctgtgtttacatcaggccaattatgtctctgtgtcagaggcacggctggtggcttgtcatcgaggcagacctggaatatttcactgattcagtatgacacatgcatcaaagaagaagtttccagaagaaatttctgaaaactccaacactgaccaga

SEQ ID NO: 98 - DNA, 408 bp, Cricetulus griseus
tactgggctcagtgtcacttaggtaggttaattgctagaatgtcctgcatcctccaactgatgtcattttattacagagaagtaagatgatctgaaacaggataatcattgcaaagccccggtgtagaagccagagctcttcatatctttagtgtttcaggttgtggttgatcactgctgtaccaaaggcaaggaagcttcagagccctggagagagcatgtgattagatatttttgtgggatttcctgtgcagcttctgtcttagacacagaagtgcatgctgccacctgatggacatcctgagaagttgcaactaaatgtttttaattgtgaaatttaactagagacaataagcaacaaaatgttagtataaacacatgaaagtgaaaaatttatatcaggacttc

SEQ ID NO: 99 - DNA, 1286 bp, Cricetulus griseus
  modified_base (84)..(934): a, c, t, g, unknown or other
tgtgcaccagtgtgttcctaagactgcggctttgttctcgtgaaggcaggagccggaactgcacgctagggcggcagcgtttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccggagttccagggtaggcggcctttgccggcgcttctcagctgctcgtttttgtgtacttggttattgtggagcccttttatctgcagcgcctgggaggattttgttttgacaagacacacatgaatcagaatgggcagacttttggtatgcatgtcgcatgtggggcaggaaagcacacacgcacgggtctagggttagcacacactaggataagggcttgtgaaggtgctagttgaagcctaaagacttaaaaaaaaaaaaatcaggcacctagtcagctgctttaccgtagtaaaactgttgcccgcttccagctcggtatccgctcctgccttttggaccgggggcg

SEQ ID NO: 100 - DNA, 2518 bp, Cricetulus griseus
agtgagtttaagacaggaaggacacattcttatgtctggaaatgaatcagagatccgcagtcactctgttttgaaaaaaaaaaaaaaaaaaaaaaaaaaaaaaaaaaaaaacatcattccctagctgaggtaacagtgtggtggttaagaaagccaacgctgggagcaaacaagcctggggtgcgaattgcagtttggccgctggcctgcagcaggaccttgggtaaactcctgtgtccttgtttgttggggtaagcagctcctccagatgtgctagggttttctgatttcaaggaagtcctctgcctgaacccatctggaagaagctaatggccgtcagctaaaccacgggaagcactaactgggcagacatgatttgactattttttcaccacacctggcatgcaggagcacttcaccagggtgctccttttttgagtgtctacaagtcctagcctcccccgccaccccgtgacaccactgagcaagagaagccacaagcctgcaatcatttctaccccacccatctctccctttctccccaccccaagagagatagtcctgcccatcacaggttagaaggtaaaagtcctctccagcacagcttggctagtcaacccctgagctcttttgtcccaagccatttgacctcccttatacaggaggctgtcacctcctgctgccctcagtgccaggccttcctccccacactaatctcagccttctccagggtttgtactacctgccttctgtctctctctgctgtcggtgtaccctttgcaatccatttctgcattttcttcacattttcttttgtaataatttgtcgatggaccttcatcctgtgagcacccttcaggcaaatattatgtcttgtcttccccctctcccagagacatgcatagtttctaagacaaactcagtgctccgcaagtgtgaccagactgcgttcgtgaaggtgatatggttgctttactttcaataatgctaatagctgggattgtttgagtgctcctgatggatcacacaatacactgggcatgtctttatttaatctcccaccaaagcatcagatctgataagtggcaaatagctaaatgaggcccagggaggtcacactggctgaccttcttgacctctggctcttgcctcaggcacaccaggcatttagttagtgctagctgctggcttgagtgggtgtcatggggcaggagctgaggaaccaccagggaagggagcactggacatagagcattgtcctcaggcccttgaaggccagctgtcattagtgtatttgcagggactgccagatccctgcgttagcatggaaagcttttcctaagtcatgccttcccagccttcttcatcctctcagggcaaggtgaagtctgagaaagcaaaggaattatcttcaccaaattctttgctgataagaccaggggagtttccagacactttcctgtgactgttccagctgagaacacagcacgaatcagcagtgactcagcagagaaaagcagggattgccgaaggttcccaggaagacaagcagctgctatacatgcacttctccaggccagggcaggaaaccacaggtggccaggagagagggcaggggatctcaagatgagagactggatgtggaaaaccacgaatggccttggagtcaggtcatgggatacagacctagtccttgctacttaccaggaatgtgaccacagctaaacaggtaaacttcccaagattagggcttccctccacagagtagagcagaaaagccactgtgaaatcaaagagttgaagtctacagtgttcaagcacagttacccatcataacagcagaaagaggatgggagggagggtaggtaaagccaccttgaactggaaaatgtagcatctgtggggccacaagctgagctagccaccttgatgtggctgggtcctggctgagaggacatactgatccctagccctggaatcctgatgtgtgtttcctgcaggttgctgtcttcaggaagggctggattactcctggctcattgtgtttgcatcatgtatgttttgcattggcaagctgaaaagtgcatgactgtgatattgtcaaggggactttccctcttgtgctcaagctggactggccacatatccccagggacgttggctgtggtgaatttcctctaaatggatagcacttccgctcatgagctctacagcccagctgacactttcccttcttcccatcccccactgccttgtgaggaaaaaaacttgtgtttacaaaaaagatgctcatatcacgagacatcatttcttcccttacaaagtgtgacaaagaaaagccatgtggccttatgctataaatgggcattttttttcaagtcaacaagtttctatgaaatcgagtgtctgggtacaaaatcccaaagcctttatggatagacagggcttggggggctgttttaagagtgtcaaaagtaaagtcttcagaggagatgagacatccatccagatgaagatg

SEQ ID NO: 101 - DNA, 911 bp, Cricetulus griseus
  modified_base (1)..(498): a, c, t, g, unknown or other
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccgggagcgggggggctggtggccggggagctgcagccaggagggctgtgacctcctggcttccgccttggtttcctggcccggggctgcggcctcccagctgggcactcctgagtcggcgtgggtgctctgcccacccaagcccctctcggcgctagcagttctgagagccgggctgcccacttcaggccccccagtgccttctctacccgcgatcggcagactctttgggaactaagagtggggttcccagccaagcagacctcgtgagccgcactgtgtgtgtgaagcgtgcacccattcagaccaaatccttcgtttcttttcgaccctccctttccgtctcacgctgttaactaaacttctatttccagataccccacaaccaaccaaccagtctgtcctatgtctt

SEQ ID NO: 102 - DNA, 295 bp, Cricetulus griseus
ggctttcttacagaggttagcccattatcagcagacatggtactggagaagtacctaagagttctacatcctgggctggaaagatgcctcagtggctaagaacactaggtggacttccaaaggtcctgagttcaatttccagcaaccacatgatggctcataaccatctgtaatgagagatcttgtgccttcttctggctttcaaggatacttgcagacagaagactaaaaggcagcaggagacactggacctagcaaagccagtgacacttcctccaacagcgccactccctgg

SEQ ID NO: 103 - DNA, 626 bp, Cricetulus griseus
tcactatgtagcccttgctgcccaggagttcactaatagaccaggttagctttgaactaagaaatccatttgcctctgtctcccgagtgctgggattaagggcatgtgcctccaagcccggccttttcctgaatttataaatatattaatctccatcacacatagaacataccacatttccttttcatcccttgtatcagtctttctcatacttactctcatgtgtgagtcatctgtgggggttacagaaaatctctgagtcagcaggcctggggagggctggagagtcgctatcgctcaccaggcagagtacgcatgcgtatgtctgttcccgcggcagcctgctacctttgcagtgtcagtagacctctggcggccacattggaaatgtgcattatttgtcacgcagtcaattttctttttctgaaagtattaaaagctccatctccccgaagccagcaaccctaccagccttgttattggcaaagccaatagtgtttactgattagttcagactcatgatttatgctttggtgccactccagacagaccacacagctggacagtggtcatcaaagaagggacatgtttagtctacataaatattgaagtgaatatgcctcttc

SEQ ID NO: 104 - DNA, 523 bp, Cricetulus griseus
ttcatggtcttcggaggggtcctcccgggaaggcatggtgggcgtagaggtcaggatggattcctgctggaggcagggagatccagaggatgcgggcgccttctgcatggaaaccggtgcagtttcgattcctggattgggtctgtgagggcgggaggtgaatcctagacttgagcaacgaaactcagccgcctcagctgtccagcggggagatacccgccgtggacccgccccccgagaggaagtgagacaggactggggcgggtctaggggcggagaggctggttctgaagtcacccttcgggagttaggaaagattggggagttttggagccacctctggtagaaccagagaggttaaccaaacaaacaaacaaacgagcaagcccagcgtgatggcatagctctgtaagcagaaacatcgcgagtttgactgctctacgagaaaaagaccctgccacgcacacaatatgcactttgggagagatgcagagttggcagaggcatttccctgttgcagatc

SEQ ID NO: 105 - DNA, 443 bp, Cricetulus griseus
gagcagccagtgctcttaactgctgagccatctctacagcccttgtgctgtattttatttgtccctccctttccttccctcattccccaccccatctttcttctctcatgggggaatctttacatacttctgcagtgattacatccagactgaattcctacaagtatagctcttgcagtctatcgcatggccccatgctgaacacatcagagtcttgggcctaggaagagattagcaatctttacccacaactccaacccccagattcccagttccccatctggtgcctcagcccagtttgtctcacttccttgattgctggcccatgtggagagctttctaatccgaaggctgccctgtgggaaggtacttcttggtttggtggttttcactcatgtattgggggctggagggtggtgtccagggctgctgtgggaacatgg

SEQ ID NO: 106 - DNA, 854 bp, Cricetulus griseus
gtgtggggcagctagctaagaaatgttggaagggtctattcctggggtgacttctgaaattttgaccttgaagatgaggatatgggttggatgcaaggcttctccgggccttttaatttcctaactttcattgcaccaaagaggaaggaaatgattattcacagtcaactgtatacaagacactgtactcggatgaaattgaacacttttcaaacttagttcctactaaagataattgtcatggtgagaccggccactagagggggcttattttgcctccagatccttcaggtcacaatcatgtcatcatcgactgtgtatttaagtgggcatatttcaaaaccgtagaataacagtgcatgtgctctctgagtaaaataaaatggctaccccataaagggtccaagaaggtaaaatgacaaagcatggtcaacccaaagatgtgtgtgtcctgatgtcacggtattagtattttaagctaaagattcatactaaagtttaataacttaattgttaagagtgaacttatactaagtgcattataaaagtaactattttccattcagggggaaagtgtaaggactttcactagatgatgtaagctcttgtacaaagccaggccagtgacctttaaaacacttgaaaggttagtcaattcagggtgctcagtcaagatctgataaactgcagctacaagccaaataaaatcaaatagaagtgagtaggacaacagtttagtaataaattatcaaatatagtatattacagtgtgagggagtaataaaaatactgagacaatataataaaagctgtatcttaatccaaacggaagtgttgtgtgttaaaagggaagc

SEQ ID NO: 107 - DNA, 517 bp, Cricetulus griseus
agcggtgctgagaacagagttctctaggcagcctttgaaaccgcctgtctgattctgaccacgcagaggagcggaaacagcaggacggaatcaaatagaagtctcaagctctagcgggccagggtctttactgcctggctcgctccgttgatcgccgggtgggggacaggagcggagagggacgccgttcggaagcgagaggaggagcccgagagcggacgggagcggagccctgcttgagggcgtggtttgagaggattcggggcggggttctgggcggggctctgggcggccccggtgcgccgctgcgttccacccttccggcacggcgttccatcagctgcttgcaccttccgcctagctcccgctttccgcgcggcggtcgctgagtttcatctttccgtggcctctgcggcgcccgagcccgcaatgtcgggccccaacggggacctaggcatgccggcggatgcgggcgcggaaggcgagaatgacagcttcggggaagcaggtgacctgg

SEQ ID NO: 108 - DNA, 407 bp, Cricetulus griseus
ctggctgtgcttcttgggcttgggtgtttgggggaaatcagatttccacatggcagacacttgaaatgcaggtattaatgtccctgaggtaggaatgtggagagaagactgctctggaagtggggtatgtaggatctgagggctagtcaagggtggttggtacttccagggactgggaccaagcctgctacatctatgttgcaggaagattggctctactctgaacatctgagtccattctatgtatacactgccctctggtggcattcaggagaattctaagcctaaggacataggcagtcaccccccctccaccctcccaccaccacaaacacactctggaccaaagggaatgtatcccatggtgcaccggacttactcacaggagccatatcccagaaggcaga

SEQ ID NO: 109 - DNA, 489 bp, Cricetulus griseus
tgatgacagtaggggaccaagggcagtttccaagtcatgattcattatattttatagtgctctaacacctagttctcagcgcattctgatggcgttggtactattagaactatcttctaacagaggggaggcagccttgacagagaccacagtgagtcccccagacctcagaagagccaattccagtgttctgttgtcatcacccctgggtgctagtctactagtggccaccagggggcagaaagaggccaggaattcagataggccgcggctttcttgaacttccctaagcctttctttgtgcaaaagggtttgatgatcaaatggtttgacctctgttttttattttgccacagaataaactcagacccagagaggaacagtgactaacccaggctacaggaccaatcacaggcagaagtgaggcctgcactctcaccctgccagcagctttgtctgggaaggtgagagacacagtgcgtaaggatt

SEQ ID NO: 110 - DNA, 1806 bp, Cricetulus griseus
aggcctcaggagacaccagcctggttggacccttggtgttgggcttccaacctctggaaccagcatcaggaagcagtgccagtgttgaatctgtggtgaccctgaccagctaatgtattatgctgctagcctgggtaactgcagttccaggactggctggggagcctctgcccaggggaaagcagcctaatgagggctgtgttctgggaaggcacactttgtgaagctgagatggtcagtatggacaggctcagaccctccaggctgaggaccataatgagcacagcattgggggtggaaggaaggaggctgctgtattcacagcagctgctaggcttcccctcccccactgcctgtgaatcccctggggctcttcagttgttgggtgtgaaaaccaatggcagaacagctgacagagccttggaaacacctacacagccggcatcagccctgggaggtgggcctatacactgggatgcacacccagctctcctgattgcttcccaagctacttgtctcacctcttccctggcaggtataaaccttcccttgctgggccctggtccaaacacctacttccatatctatttagaaggtccctaccccaggtgccttatccacagccaccctcttcatgctgtcacagaacttcacctgtgcccaactccctatagccaatgatgccccatggccttgtgcatgctcagctgggttcagatttagagagaccgtgggtaactaaaaagaccaggaacataggctcagcccaaacagcctggagccctcctgagccagacactgcccacccacaatcctgtcatatgtcctgtatacaagtggaagtattcagcttttgacctgatacttgggtcatgtaaatctgggcatctggccagctgtggccagcaccctctctcctgggtgttctctcatacatgggctgcctggctcctgaggtcctgtctagtaccacacattacttctaaacatgagtgtccctcccactgggtcatgcaccagagaggtcttcagccatgtgcagcaccttccaatttagtcctttctgggctctgagcaccccaccctcacccctcccccaggagcccattctcttgtttacacttaggcctgcctggttatcttcccaagcttcctccctgatgaagtcagcagaactaattgacatcagaaactggaacgcagctgggcggggccagggaatctggcaggccatacccacagtgtgccaggaatgctgccctctaccctccaccatcgaggaagagccttccaagcactacctgtgaaagcggactgcctggaagactcatcctgggcttggaggcccagtgagtgtttgagaggcaggctcatggttcacaaaatgcacaactctaacttgccttcaggagtcagacaatggaaacacccattatctattccattttacagatggaaaatggagactcaagaaaccaaactgactcactcaagttacaggaaagtggtttagatgggcaggtacttagagctctcctgcccccaacacctcagagttgaaatggaaagttacaaagtagcataagctgcagtcaatgctgtcagtggataatgcttgcttcttgggaatcccactggaacagccagtccacattttagggcagacaggacaggaagattctggcagaagtgtcttctctcatctaagaaatcatgtgacaaagccaggtaaatctgaacaggaaagcctttggagcagatctggccagaatagtcctgaactggacacagggatcagggctggtt

SEQ ID NO: 111 - DNA, 881 bp, Cricetulus griseus
agccattagctgggcaaactaacatcttgcagagatgacagatgggagagagaggggtgagttcctcccatctgagctcgctcccggcacagggaagaatcctcacaacaatgggctaaaatcctttcctaagacaagccctctgggccccctgtcagaaaccccactaatcgcttcccaagctccccaccctgtagctgaagcttggttcacaacatttacagctaagtacattattaaaagtcaaaccaaacaacgtttgccagtccctgggggaatgtggcgaggaatgaataattctgaaaagatcctggcatctgtgtgcattaaccacaaatggccattaagtcagagcagcacagggagcagcctgcaggctgcagagctcacacccatgagaagtgctggttctccccctttccaggaggaggccaaggcctggtccccacccacagtcagcctgagggccaccttctctccctttgatccctagaatggaacaacctaattccttctcagcagcttcaccaaacactgcagccagtcactgccctggacttctggggctcctgtagctccctccaccccaacaaatagtctgagaggatgggcccttgcagccagctccagtgtgtggtggcctgcagcacacactgcctctcaccaaagatgggagcatagtctccatggcagctggccctcgcctcttccgtctctcacttcacatgcttggggagtgactgggattctggatgctgtgttgtcagggagatggtcatacgggcttttgatatccttatatagtcgtgacattggctaattacctagtctgcctgaatccagctttcttatttgtcaaataggaccaagcctcttctgct

SEQ ID NO: 112 - DNA, 327 bp, Cricetulus griseus
ggccatcctttaccgtggacccggatcaagatcaaaacatgggcacctccgactgcacgcatgcgcatgtgccaggatgactactgcgtgtacagactggaaacctgctgcgcaatgcggggttcagattcacaacctgtgcctgattgtgctgatgccggagcgccctctaccgaggcgcccgaggaataggcggaggcacttgcggaggaaaagacaggaacacatttacagagggcttgaaaagctggatcatccccgcctgggctgcctgtggaagcatcaacactcccagggactcactgcttagctaagcgggcaggggtg

SEQ ID NO: 113 - DNA, 586 bp, Cricetulus griseus
catgctgggtgatgatggccaggctggctcctcctctcctgattctgagcccaggtgacccctggctggtagaagcctgtctctgccctgtgttctgacctcacactggctgtctgccagtgtctccaagcgatgcacttcactgctgtcgcaggaagcccaaatttgatcaagcccaacctcatcttacggagtgcagtctgcacagcttcccgttcctgggcggtggggctggggtggtggagggtggaggagggacggacagggatgggacaggacacaggatgacaaccacatgactcagaattctccagcctgtgagtttggggtagtcactcgcacctgccattgaggcagaagtataaacaacatgccagtatctgtgcgagtctgtgtggtcccatagaaacatgccataatcaccgtgtgtctaatgagtcatcagcattgtgattagtctaatgttacatcattacagggtttgtggttcagtttactgcagtatgggaggatggcgctcaagacccagcgcaggatggaaacaccctaaccacacaccagctcatcactgggtgcttctggacag

SEQ ID NO: 114 - DNA, 673 bp, Cricetulus griseus
  modified_base (77)..(93): a, c, t, g, unknown or other
  modified_base (270)..(424): a, c, t, g, unknown or other
gtaggggtctccgtcccagaaacagtggggaaagacaccccaggcaaggaggggccgctccccggtggctttgggannnnnnnnnnnnnnnnnctcaccggtcagtgcgtcctggagcgccctgctgaacaccttgagtctttgctcgctcacgcagccccgcgtcctcaggaggctggaaaggaagccgacggcggcggcaatctccgggagcatgtctgttctcttcccgtggctcatgccgggcggctgggtaaggagctgtggtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcggtggaagacgggcgggagagggaccaggacctgggcagaggaagcgcgccacaccgggcatgcccggacgtcgcctctgcacagttccaaggcagcggttgcctgccacctcctccaggatgtcctcgccgatgctacttctcccacgcctccccaaaacagacactctggcgccaggtcatctctgccactctctcccagctggtcagaaccggctcctcatgttttcggcaatggggattgtag

SEQ ID NO: 115 - DNA, 389 bp, Cricetulus griseus
tcacttaatctcaacgatatggttgcctagacttgcatggtgacaccagcggctgagaaggtctcatactccttaacgtgctttaagaggagtctgtaggctaaggcaaccccttccagcagtgagcagtgctttacgtgattgtgccagcaggtggcgcccgagcaccaccgtttctaaatggagtcccaaccatcaggcccaacttgttactactggtccttgatatctggaaagacaaatattgagtgaaaggaaagtctttggagatactttacccagggtgcgatcttttctggactcacggtaacgagcacttttcgtgctggcccctagcgcgacactaactgagtccccctggtatcagagcctcctgtaccccctcttct

SEQ ID NO: 116 - DNA, 531 bp, Cricetulus griseus
ctgctccttttaccagctttttcccccctcttgcattttttatttttatgaaaggatgtcattaaggcttttgtctgcgctgtttttgtttcagaacttttcccacaatcctattttttgttgaggaatccgctcctttgcccagctgtgggtccgggtcccacagctgtgttgatctaagactcagccccagacagcctggcgccagggtgggacgtcatgcgttcacacagggatgcgtgtcccaggcaacctttcctcgggcagtcaccggccggcgactgcctctcccaccaatgaacgcccgacacccgagataggggcggggcctgcaccaccccagccggcgagcggcttgtgaatctcagattctgagaccttgataacgtcccttgaattggggccctttaaagtccccctttctcttttagttcttttgtttatcccgtttctttccaccttccttctttctgtgtgtagataagttttctttttccttttcccctcttttaactctctcctagtgtcagg

SEQ ID NO: 117 - DNA, 856 bp, Cricetulus griseus
tctctgcctccagagcgttggcatgggccactatgatggaataaagcctggaagttcgtatctttggcctgtacgtttaagtaatttacactatcttctctgacaaggactggggggggggtcttgaaaggactgagcttgagacttcagaaagagaacagatgccaagacgtgcaaaacactttagaaatgcaggcaaaccctttcatttcacaactaacaaaaccgacgatcaggattgggggggaaccctccctcccgagttaaccacggggtcagcaactcagtggggcctaagacaggttcccttgcccggctgagtgctttttccattataccggctgcgcctcagtaaccccacccccgaaagtgctagggcagctccctcccagtgcccaggcggggtggggcccctccgtggcatggaacaaaccgggaaaaccacatattccaactcccagcacacaaaggctggttggccatgggggcggggtgggaggaccggacctaagtgtagctcactcctgagattctgcgcggggctcctagattgcctgctctaaggagccaccctactttgtcctgttcggtgtcccctgggggggggagggtggcggcaagacctcccccaaataaatggcctgttctggctccagcgtcccaccccctagctgcctacggaaatctaggagggacggtcctctcctttccgctttcccatccgcccccggagcgtcgcacagctgggttgacttgggtcgcctccccctctccacttgcggtgtgtcctactccaggcgctttccttccttccacagccccccactttggaaggaagacacgctccaaagctagc

SEQ ID NO: 118 - DNA, 1001 bp, Cricetulus griseus
acccttccttccaactccagtgagctcacaaggggactacctttccctctggttcccctaaagatcacgctgaagttctgctgacacgccaatccccaccctgcgtatatggactacagcaggtgtcactcagcttcgggtcccccttcttcggatgctcctcccacagggccgctccaaccctcacgcgaacgccgtgtgttcggggacagactaaaagcaaaaacaggctattgggttccgtaccgagaattcgggccgggcgtcagggtcgccgcccgccaactcgcgccgtctcggtctcggggcagcagcgcagacgaccaagactggggctcggacaccggtcccgagcaaacaggacagccaacacaggtccttgctgccggcgcctcctccgccagagcggctcgcagaccggaagttttccgcggtcggagggaggaaccggaaggaaccatccgtgcctttggtggatgctggcccggctcagctcgacccagccctacaccgctgcgtacttagcgtcgggaagctctcacgagccacaaaagctctcatgatgctccatcacttaagtgctagttttttcttgatttcctttcgggccattagactcggtgagaagaataaccccctcctttggacgagtccattcactcctaccagcgacaacgttgtttggactgggacaactcacttccgccatcgagacaagagggaaagaagggacgggtgaactctccgctgcctctggtggccaagctgagtcatcggcggagagctctcccctgcgaccaaggcgtgatccccggcgggtgtgacagccctaaaacaacagggatcccaagaccccttaacatttgaggggctccctggagatgagcgcacgccggtgtgctgctgcacgaggccgataggtagccgaatttgtgctggcccaaagtgcgtaacagtcaggctcactgggcttagacccacaagaagatttcgaaaaagga

SEQ ID NO: 119 - DNA, 403 bp, Cricetulus griseus
ccctgtgtgtgtgcctttgtatatctgtgttccacacaggaccctcacaagactgtgggctagcctatatgcttcatgatgcagactgggacaggctggccctctgcgagctcacgtctccagaagccctcaggcaggaagaaggcctcacctctgcctggctggtcctcagcctggctcagccgtgctccagccctgtctggggccaggcagcctttgaggacatagctgaggtttggcggtgccgcctctccggggctgagtcagcatagggagggaatcaggcctccactgcagaagcagttgggatcttggaggccagggtccactagcccacttgggcttcaagagggtggggtgtgctcagtgcacccagctgtaggcagctggactctgtaggaca

SEQ ID NO: 120 - DNA, 1009 bp, Cricetulus griseus
tgctccgggtcttcaagcttaggatccagcacgactcacaggatcaggaggagacagaaagcaaggaggaaggaaaaagggagagaaggccagagagaagtggaaggagcatgaaaaaggcttgggaaagaggaaggaagaggagagctggaaggggagacggaacaatgaaggtctcaggccctccgaggagcacggttccggctgcacgtccgtcccgcccgcctccggccaccagctgctcgctgcacagcgtgtgggcgctgagtcactgtcccagcacagggagcccgcgtgcaggtcggggggagcccggttggcatctgcaaactgctttgttttttgttttttgttttttgtttctttcctctttcattaaggctgtgtgctgactcccacgtaggagaacttattaaaccgcacgtactactccaaaactcttgggaaccacgcatgatgaccagcagggggtgccacagccccgtttatgagcgctagggcccagtgaagacttggggctactagcatgtttcccagagacgtgcgagcattttgtaggggtaaactttttactttaaatcccgcagctgcagcataaactctctgcttagaaagcctagaagatcgccataagtctccaaacagcgccttagcgcaggttcggggagcaacccaataaaattgggagtgtattgcgtgcgcaaccaagggagcccaggctcgcctaacaactagggttcgtcctacccaaccgggtctggtcgccactcgaggcggtgaacttgggtatctcagagaaatcttgactcccacccaggacacgcgctctgtgcatccacatgtacaatggagctcatgacagataaacccatgcatagagagattcccaaaacaagaggcaagatcccaacactcccaggcgatccaaaccaagacaatctttccctagaagcatgaaccgagaatgaatcagcattgagacagtttattggtttgctttttctcagat

SEQ ID NO: 121 - DNA, 434 bp, Cricetulus griseus
gattacgcagttgcttctccgacatcacccatcctttttgaatcccttcacaaacggggagcttctgtgagggatcccaagagagatctttgtgatctttaaggtgttcagtgaatgcaaattaagtgttcctactgtataatttctcggagcgtgtttagttgtcttcgtggttggttggtaaaccagagcctacagggtttggtcagttgctgttggctcctagagagagtgcgtgtccccaacttccaacagggggcactcctgagttgtttaacgtgaggctgaagatgggtttccaaacacatttcatctcccgacgcaagtgaatgtcttgtatctagctatcctacagggtgaatgagaaattgacaatgagagaagttaagtcacttacttagatcactgactaaatagcagacctggttcaaact

SEQ ID NO: 122 - DNA, 689 bp, Cricetulus griseus
  modified_base (396)..(516): a, c, t, g, unknown or other
gcttggtgcaagaaggggagaggagggcttttaaaagctcagactccccgtccccacctgcgccgcgggaaaacctggggacgcgtgggggcggggcgcacctggatgtcctcgatcatgtcccccgagaagagctctcgactcagcagagcgtcccagagctcggcgacctgcagctcgctcaccaggcgcacccgacatcgccgcaggagctgccggtccgcttcgtccatggccggccgattgcggaccccgagttccgtccgcttccggctcgggatcctgcccgggcgctcacgccacgcccccatatcgcccgggcaagggatcccgccccgtggaggccctgcaggccaacagtctcctcgtcatcaggctccgcccggccctaaggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgctcctccgccccgcccctagggccccccagttccacagctcagcccctccccgggaccggttctcaggctccaccctccaacgtttatcactgcggcgctattgcatcctgaccctaccctgcctctcccgtcctgatcctgcccccgggccccgcctcctgcgaccgtgc

SEQ ID NO: 123 - DNA, 508 bp, Cricetulus griseus
aggctagctaaggctggccttgaacttctgatcctcctgcctccatctcccaaatgcggggattgcaagcatgtgtcaccatgactgttgtgtgcagtgctaaggcttaggcagcggagctacattccaagtcttggacctccctgattttctaggataaaatacccaatccatcccttggctttcaaggccgggcctgaatatataaagcatccagcagggggcgctctagagacgtcctggagaggtagcccctaacctccatgttgtttccccattcctgggctctcccctcgccaccacctttgctatgaaggatgtgtgtggggtgactcattagagccacacttcagagtatatccagagcctgctggagggttgcccagctggaatgtctgcaaggagcctgctctttgctgttctgtttcatctctgcctgcagtgatcagcacggcctgtctggcacctgtgagctgattgggaagggaaggagggggagctgggctgc

SEQ ID NO: 124 - DNA, 435 bp, Cricetulus griseus
acagtaacaacgttgccattatctacctgatgtccaccacactgagttctccttctattcttaggactggcctggagctcctggtcagcccactttcagtgcctggtgaaatgagagcagccgcttcttgcacagacgtatttagaatgaacaggatgagatagcaaatatttgaaatgcctatgagcagctgtggagggccagcttcccaaaactggaattcttaaccattaccagctcagtcttgagccaaagcagctgcgtgggagaccagaggcacttgtctagtggtgacaagcatgtgctgtgtgaactcaaatggctgcatgagctctggtgtcctcactggctgcaaggctatttaaaacttcagagcagcagacagcagatgcctgacacttcctatctctgcttcaaaatagagagaattaagac

SEQ ID NO: 125 - DNA, 697 bp, Cricetulus griseus
cttgcatgaagacttaggaatttaggtccacatgcttgtgtggcaaggattttatcaagtgagccatctccccaacccttaagttttcttagaattgtgtcatcttataaatgtatttatatgcttctgtcaagatgctggttggttatttaattaaacattcactttatagacaaaagatgacaggttctagaaaagccgattgatcaaagtcaccgagctaagatttaatgatctattgttcccaaagtcctcgctgacagagtgcatgaattccatgaggcacaaatatgtgtgagtaactgtggactacaggagaggcctctgactcatctggctagggtcattactttgcaggcacttgggctatccacaggaaagccagggaaggcaggggtttttctaaccattgaagagggatatcagaggaatttggggacattcttcttggatgttatttattaaataagcatttattaaacatttattatgagtgtactgtgattaaaaacataaaaacaatagtattaattcagtcggggatacccatgtcaggtggggatacctgtaatgttttcatagatgaggagtagctaaagtggtcttttagtgctactcaacatacatttggaagacaccatcagaagaagagggagagatgggagtagatttcgctttaagaagacagtcacagggg

SEQ ID NO: 126 - DNA, 34 bp, Escherichia coli
ataacttcgtataatgtatgctatacgaagttat

SEQ ID NO: 127 - DNA, 34 bp, Artificial Sequence
  source: /note=“Description of Artificial Sequence Synthetic oligonucleotide”
ataacttcgtataatgtatactatacgaagttat

SEQ ID NO: 128 - DNA, 34 bp, Artificial Sequence
  source: /note=“Description of Artificial Sequence Synthetic oligonucleotide”
ataacttcgtataaagtatcctatacgaagttat

SEQ ID NO: 129 - DNA, 34 bp, Artificial Sequence
  source: /note=“Description of Artificial Sequence Synthetic oligonucleotide”
ataacttcgtataatgtgtactatacgaagttat

SEQ ID NO: 130 - DNA, 34 bp, Artificial Sequence
  source: /note=“Description of Artificial Sequence Synthetic oligonucleotide”
ataacttcgtataggatactttatacgaagttat

SEQ ID NO: 131 - DNA, 34 bp, Artificial Sequence; identical to SEQ ID NO: 126
  source: /note=“Description of Artificial Sequence Synthetic oligonucleotide”

SEQ ID NO: 132 - DNA, 48 bp, Saccharomyces cerevisiae
gaagttactattccgaagttcctattctctagaaagtataggaacttc

SEQ ID NO: 133 - DNA, 48 bp, Artificial Sequence
  source: /note=“Description of Artificial Sequence Synthetic oligonucleotide”
gaagttactattccgaagttcctattctctagatagtataggaacttc

SEQ ID NO: 134 - DNA, 48 bp, Artificial Sequence
  source: /note=“Description of Artificial Sequence Synthetic oligonucleotide”
gaagttactattccgaagttcctattctctacttagtataggaacttc

SEQ ID NO: 135 - DNA, 48 bp, Artificial Sequence
  source: /note=“Description of Artificial Sequence Synthetic oligonucleotide”
gaagttactattccgaagttcctattcttcaaatagtataggaacttc

SEQ ID NO: 136 - DNA, 48 bp, Artificial Sequence
  source: /note=“Description of Artificial Sequence Synthetic oligonucleotide”
gaagttactattccgaagttcctattctctagaaggtataggaacttc

SEQ ID NO: 137 - DNA, 48 bp, Artificial Sequence
  source: /note=“Description of Artificial Sequence Synthetic oligonucleotide”
gaagttcctattccgaagttcctattcttcaaaaggtataggaacttc

SEQ ID NO: 138 - DNA, 48 bp, Artificial Sequence
  source: /note=“Description of Artificial Sequence Synthetic oligonucleotide”
gaagttcctattccgaagttcctattcttcaaaaagtataggaacttc

SEQ ID NO: 139 - DNA, 48 bp, Artificial Sequence
  source: /note=“Description of Artificial Sequence Synthetic oligonucleotide”
gaagttcctattccgaagttcctattcttcaataagtataggaacttc

SEQ ID NO: 140 - DNA, 48 bp, Artificial Sequence
  source: /note=“Description of Artificial Sequence Synthetic oligonucleotide”
gaagttcctattccgaagttcctattctatcagaagtataggaacttc

SEQ ID NO: 141 - DNA, 48 bp, Artificial Sequence
  source: /note=“Description of Artificial Sequence Synthetic oligonucleotide”
gaagttcctattccgaagttcctattcttataggagtataggaacttc

SEQ ID NO: 142 - DNA, 48 bp, Artificial Sequence
  source: /note=“Description of Artificial Sequence Synthetic oligonucleotide”
gaagttactattccgaagttcctatactttctggagaataggaacttc

SEQ ID NO: 143 - DNA, 48 bp, Artificial Sequence
  source: /note=“Description of Artificial Sequence Synthetic oligonucleotide”
gaagttactattccgaagttcctatactctccagagaataggaacttc

SEQ ID NO: 144 - DNA, 48 bp, Artificial Sequence
  source: /note=“Description of Artificial Sequence Synthetic oligonucleotide”
gaagttactattccgaagttcctatactatctacagaataggaacttc

SEQ ID NO: 145 - DNA, 48 bp, Artificial Sequence
  source: /note=“Description of Artificial Sequence Synthetic oligonucleotide”
gaagttactattccgaagttcctatactctctggagaataggaacttc

SEQ ID NO: 146 - DNA, 48 bp, Artificial Sequence
  source: /note=“Description of Artificial Sequence Synthetic oligonucleotide”
gaagttactattccgaagttcctatactatcttgagaataggaacttc

SEQ ID NO: 147 - DNA, 32 bp, Artificial Sequence
  source: /note=“Description of Artificial Sequence Synthetic oligonucleotide”
taactttaaataatgccaattatttaaagtta

SEQ ID NO: 148 - DNA, 32 bp, Artificial Sequence
  source: /note=“Description of Artificial Sequence Synthetic oligonucleotide”
taactttaaataattggcattatttaaagtta

SEQ ID NO: 149 - DNA, 52 bp, Artificial Sequence
  source: /note=“Description of Artificial Sequence Synthetic oligonucleotide”
catcagggcggtcaggccgtagatgtggaagaacggcagcacggcgaggacg

SEQ ID NO: 150 - DNA, 52 bp, Artificial Sequence
  source: /note=“Description of Artificial Sequence Synthetic oligonucleotide”
atgtggtcctttagatccactgacgtgggtcagtgtctctaaaggactcgcg

SEQ ID NO: 151 - DNA, 52 bp, Artificial Sequence
  source: /note=“Description of Artificial Sequence Synthetic oligonucleotide”
catcagggcggtcaggccgtagatgtgggtcagtgtctctaaaggactcgcg

SEQ ID NO: 152 - DNA, 52 bp, Artificial Sequence
  source: /note=“Description of Artificial Sequence Synthetic oligonucleotide”
atgtggtcctttagatccactgacgtggaagaacggcagcacggcgaggacg

SEQ ID NO: 153 - DNA, 34 bp, Artificial Sequence
  source: /note=“Description of Artificial Sequence Synthetic oligonucleotide”
acaacttcgtataatgtatgctatacgaagttat

SEQ ID NO: 154 - DNA, 34 bp, Artificial Sequence
  source: /note=“Description of Artificial Sequence Synthetic oligonucleotide”
taccgttcgtatagtatagtatatacgaagttat

SEQ ID NO: 155 - DNA, 34 bp, Artificial Sequence
  source: /note=“Description of Artificial Sequence Synthetic oligonucleotide”
taccgttcgtatagtatagtatatacgaacggta